SEQUENCE LISTING (84 sequences)

SEQ ID NO: 1 - protein, 100 aa, Homo sapiens
PSAPVVSGPAARATPQHTVSFTCESHGFSPRDITLKWFKNGNELSDFQTNVDPVGESVSYSIHSTAKVVLTREDVHSQVICEVAHVTLQGDPLRGTANLS

SEQ ID NO: 2 - protein, 95 aa, Homo sapiens
PTLEVTQQPVRAENQVNVTCQVRKFYPQRLQLTWLENGNVSRTETASTVTENKDGTYNWMSWLLVNVSAHRDDVKLTCQVEHDGQPAVSKSHDLK

SEQ ID NO: 3 - protein, 17 aa, Homo sapiens
KGKHLCPSPLFPGPSKP

SEQ ID NO: 4 - protein, 25 aa, Homo sapiens
YVTVSSQDPAEPKSPDKTHTCPPCP

SEQ ID NO: 5 - protein, 5 aa, Artificial Sequence
  linker
GGGGS

SEQ ID NO: 6 - protein, 7 aa, Artificial Sequence
  linker
GGGGSVP

SEQ ID NO: 7 - protein, 7 aa, Artificial Sequence
  linker
GGGGSAK

SEQ ID NO: 8 - protein, 7 aa, Artificial Sequence
  linker
VSGGGGS

SEQ ID NO: 9 - protein, 6 aa, Artificial Sequence
  linker C1
ETIRVP

SEQ ID NO: 10 - protein, 42 aa, Artificial Sequence
  CAR spacer XS
YVTVSSQDPAEPKSPDKTHTCPPCPKGKHLCPSPLFPGPSKP

SEQ ID NO: 11 - protein, 132 aa, Artificial Sequence
  CAR spacer 1S
YVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLS

SEQ ID NO: 12 - protein, 129 aa, Artificial Sequence
  CAR spacer 2S
YVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVS

SEQ ID NO: 13 - protein, 154 aa, Artificial Sequence
  CAR spacer X1S
YVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSGGGGSKGKHLCPSPLFPGPSKP

SEQ ID NO: 14 - protein, 146 aa, Artificial Sequence
  CAR spacer X2S
YVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKP

SEQ ID NO: 15 - protein, 235 aa, Artificial Sequence
  CAR spacer M
YVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVS

SEQ ID NO: 16 - protein, 252 aa, Artificial Sequence
  CAR spacer XM
YVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKP

SEQ ID NO: 17 - protein, 339 aa, Artificial Sequence
  CAR spacer L
YVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVS

SEQ ID NO: 18 - protein, 356 aa, Artificial Sequence
  CAR spacer XL
YVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKP

SEQ ID NO: 19 - protein, 108 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKR

SEQ ID NO: 20 - protein, 120 aa, Homo sapiens
EVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 21 - protein, 20 aa, Artificial Sequence
  linker
GGGGSGGGGSGGGGSGGGGS

SEQ ID NO: 22 - protein, 248 aa, Artificial Sequence
  scFV anti-CD19
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 23 - protein, 27 aa, Homo sapiens
FWVLVVVGGVLACYSLLVTVAFIIFWV

SEQ ID NO: 24 - protein, 41 aa, Homo sapiens
RSKRSRLLHSDYMNMTPRRPGPTRKHYQPYAPPRDFAAYRS

SEQ ID NO: 25 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 491 aa, Artificial Sequence
  CAR XS
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 586 aa, Artificial Sequence
  CAR 1S
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 583 aa, Artificial Sequence
  CAR 2S
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 603 aa, Artificial Sequence
  CAR X1S
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSGGGGSKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 595 aa, Artificial Sequence
  CAR X2S
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 689 aa, Artificial Sequence
  CAR M
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 701 aa, Artificial Sequence
  CAR XM
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 793 aa, Artificial Sequence
  CAR L
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 805 aa, Artificial Sequence
  CAR XL
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 129 bp, Artificial Sequence
  CAR spacer XS
agctacgtgaccgtgagcagccaggaccccgccgagcccaagagccccgacaagacccacacctgccccccctgccccaagggcaagcacctgtgccccagccccctgttccccggccccagcaagccc

SEQ ID NO: 36 - DNA, 399 bp, Artificial Sequence
  CAR spacer 1S
agctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagc

SEQ ID NO: 37 - DNA, 390 bp, Artificial Sequence
  CAR spacer 2S
agctacgtgaccgtgagcagccaggaccccgccgagcccaagagccccgacaagacccacacctgccccccctgccccggaggaggaggatctgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagc

SEQ ID NO: 38 - DNA, 465 bp, Artificial Sequence
  CAR spacer X1S
agctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcggaggaggaggatctaagggcaagcacctgtgccccagccccctgttccccggccccagcaagccc

SEQ ID NO: 39 - DNA, 441 bp, Artificial Sequence
  CAR spacer X2S
agctacgtgaccgtgagcagccaggaccccgccgagcccaagagccccgacaagacccacacctgccccccctgccccggaggaggaggatctgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagccc

SEQ ID NO: 40 - DNA, 707 bp, Artificial Sequence
  CAR spacer M
agctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagc

SEQ ID NO: 41 - DNA, 759 bp, Artificial Sequence
  CAR spacer XM
agctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagccc

SEQ ID NO: 42 - DNA, 1020 bp, Artificial Sequence
  CAR spacer L
agctacgtgaccgtgagcagccaggaccccgccgagcccaagagccccgacaagacccacacctgccccccctgccccggtggcggtggaagtgtgccccccaccctggaggtgacccagcagcccgtgagagccgagaaccaggtgaacgtgacctgccaggtgagaaagttctacccccagagactgcagctgacctggctggagaacggcaacgtgagcagaaccgagaccgccagcaccgtgaccgagaacaaggacggcacctacaactggatgagctggctgctggtgaacgtgagcgcccacagagacgacgtgaagctgacctgccaggtggagcacgacggccagcccgccgtgagcaagagccacgacctgaaggtgagcggcggaggcgggagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagc

SEQ ID NO: 43 - DNA, 1071 bp, Artificial Sequence
  CAR spacer XL
agctacgtgaccgtgagcagccaggaccccgccgagcccaagagccccgacaagacccacacctgccccccctgccccggtggcggtggaagtgtgccccccaccctggaggtgacccagcagcccgtgagagccgagaaccaggtgaacgtgacctgccaggtgagaaagttctacccccagagactgcagctgacctggctggagaacggcaacgtgagcagaaccgagaccgccagcaccgtgaccgagaacaaggacggcacctacaactggatgagctggctgctggtgaacgtgagcgcccacagagacgacgtgaagctgacctgccaggtggagcacgacggccagcccgccgtgagcaagagccacgacctgaaggtgagcggcggaggcgggagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagccc

SEQ ID NO: 44 - DNA, 1476 bp, Artificial Sequence
  CAR XS
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggtggtggtggttctggtggtggtggttctggcggcggcggctccggtggtggtgggtccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccaagggcaagcacctgtgccccagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 45 - DNA, 1761 bp, Artificial Sequence
  CAR 1S
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggtggtggtggttctggtggtggtggttctggcggcggcggctccggtggtggtgggtccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcggtggtggtggttccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 46 - DNA, 1752 bp, Artificial Sequence
  CAR 2S
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggtggtggtggttctggtggtggtggttctggcggcggcggctccggtggtggtgggtccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggaggaggaggatctgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcggtggtggtggttccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 47 - DNA, 1812 bp, Artificial Sequence
  CAR X1S
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggtggtggtggttctggtggtggtggttctggcggcggcggctccggtggtggtgggtccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcggaggaggaggatctaagggcaagcacctgtgccccagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 48 - DNA, 1788 bp, Artificial Sequence
  CAR X2S
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggtggtggtggttctggtggtggtggttctggcggcggcggctccggtggtggtgggtccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggaggaggaggatctgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 49 - DNA, 2070 bp, Artificial Sequence
  CAR M
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggtggtggtggttctggtggtggtggttctggcggcggcggctccggtggtggtgggtccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcggtggtggtggttccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 50 - DNA, 2106 bp, Artificial Sequence
  CAR XM
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggtggtggtggttctggtggtggtggttctggcggcggcggctccggtggtggtgggtccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggaggaggaggatctgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 51 - DNA, 2382 bp, Artificial Sequence
  CAR L
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggtggcggtggaagtgtgccccccaccctggaggtgacccagcagcccgtgagagccgagaaccaggtgaacgtgacctgccaggtgagaaagttctacccccagagactgcagctgacctggctggagaacggcaacgtgagcagaaccgagaccgccagcaccgtgaccgagaacaaggacggcacctacaactggatgagctggctgctggtgaacgtgagcgcccacagagacgacgtgaagctgacctgccaggtggagcacgacggccagcccgccgtgagcaagagccacgacctgaaggtgagcggcggaggcgggagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcggcggtggcggcagcttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 52 - DNA, 2418 bp, Artificial Sequence
  CAR XL
atggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagctacgtgaccgtgagcagccaggaccccgccgagcccaagagccccgacaagacccacacctgccccccctgccccggtggcggtggaagtgtgccccccaccctggaggtgacccagcagcccgtgagagccgagaaccaggtgaacgtgacctgccaggtgagaaagttctacccccagagactgcagctgacctggctggagaacggcaacgtgagcagaaccgagaccgccagcaccgtgaccgagaacaaggacggcacctacaactggatgagctggctgctggtgaacgtgagcgcccacagagacgacgtgaagctgacctgccaggtggagcacgacggccagcccgccgtgagcaagagccacgacctgaaggtgagcggcggaggcgggagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 53 - protein, 247 aa, Homo sapiens
  HER-2 scFv
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVS

SEQ ID NO: 54 - protein, 689 aa, Artificial Sequence
  Her2 CAR M
MEFGLSWLFLVAILKGVQCSRDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSYVTVSSQDPAEPKSPDKTHTCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - DNA, 2070 bp, Artificial Sequence
  HER-2 CAR M
atggagttcggcctgagctggctgttcctggtggccatcctgaagggcgtgcagtgcagcagggacatccagatgacccagagccccagcagcctgagcgccagcgtgggcgacagggtgaccatcacctgcagggccagccaggacgtgaacaccgccgtggcctggtaccagcagaagcccggcaaggcccccaagctgctgatctacagcgccagcttcctgtacagcggcgtgcccagcaggttcagcggcagcaggagcggcaccgacttcaccctgaccatcagcagcctgcagcccgaggacttcgccacctactactgccagcagcactacaccaccccccccaccttcggccagggcaccaaggtggagatcaagaggggcggtggaggttccggcggtggcggttccggaggcggtgggtcaggaggtggaggctccgaggtgcagctggtggagagcggcggcggcctggtgcagcccggcggcagcctgaggctgagctgcgccgccagcggcttcaacatcaaggacacctacatccactgggtgaggcaggcccccggcaagggcctggagtgggtggccaggatctaccccaccaacggctacaccaggtacgccgacagcgtgaagggcaggttcaccatcagcgccgacaccagcaagaacaccgcctacctgcagatgaacagcctgagggccgaggacaccgccgtgtactactgcagcaggtggggcggcgacggcttctacgccatggactactggggccagggcaccctggtgaccgtgagcagctacgtgaccgtgagcagccaggaccccgccgagcccaagagccccgacaagacccacacctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcggcggtggcggcagcttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 56 - protein, 220 aa, Artificial Sequence
  CAR spacer M1
ESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLK

SEQ ID NO: 57 - protein, 239 aa, Artificial Sequence
  CAR spacer XM2
ESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKP

SEQ ID NO: 58 - protein, 256 aa, Artificial Sequence
  CAR spacer XM3
ESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRESKYGPPCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKP

SEQ ID NO: 59 - protein, 256 aa, Artificial Sequence
  CAR spacer M4
ESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRESKYGPPCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSESKYGPPCPPCPGGGGS

SEQ ID NO: 60 - protein, 133 aa, Artificial Sequence
  CAR spacer 2S5
ESKYGPPCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSESKYGPPCPPCPGGGGS

SEQ ID NO: 61 - protein, 208 aa, Artificial Sequence
  CAR spacer M6
PSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGS

SEQ ID NO: 62 - protein, 676 aa, Artificial Sequence
  CAR M1
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 688 aa, Artificial Sequence
  CAR XM2
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 705 aa, Artificial Sequence
  CAR XM3
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRESKYGPPCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 705 aa, Artificial Sequence
  CAR M4
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSESKYGPPCPPCPGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRESKYGPPCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSESKYGPPCPPCPGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 582 aa, Artificial Sequence
  CAR 2S5
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSESKYGPPCPPCPGGGGSVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSESKYGPPCPPCPGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 664 aa, Artificial Sequence
  CAR M6
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSGGGGSAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSGGGGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 666 bp, Artificial Sequence
  CAR spacer M1
gagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagc

SEQ ID NO: 69 - DNA, 717 bp, Artificial Sequence
  CAR spacer XM2
gagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagccc

SEQ ID NO: 70 - DNA, 768 bp, Artificial Sequence
  CAR spacer XM3
gagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggaatccaaatacggaccaccatgcccaccatgcccaggcggaggcggtagtgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagccc

SEQ ID NO: 71 - DNA, 768 bp, Artificial Sequence
  CAR spacer M4
gagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggaatccaaatacggaccaccatgcccaccatgcccaggaggtggcggaagtgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcgaatccaaatacggaccaccatgcccaccatgcccaggcggtggcggcagc

SEQ ID NO: 72 - DNA, 399 bp, Artificial Sequence
  CAR spacer 2S5
gagagcaagtacggccccccctgccccccctgccccggtggcggtggaagtgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcgaatccaaatacggaccaccatgcccaccatgcccaggcggtggcggcagc

SEQ ID NO: 73 - DNA, 645 bp, Artificial Sequence
  CAR spacer M6
ggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcggcggtggcggcagc

SEQ ID NO: 74 - DNA, 2037 bp, Artificial Sequence
  CAR M1
gccaccatggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagcgagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcggcggtggcggcagcttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 75 - DNA, 2073 bp, Artificial Sequence
  CAR XM2
gccaccatggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagcgagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 76 - DNA, 2124 bp, Artificial Sequence
  CAR XM3
gccaccatggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagcgagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggaatccaaatacggaccaccatgcccaccatgcccaggcggaggcggtagtgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcaagggcaagcacctgtgccccagccccctgttccccggccccagcaagcccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 77 - DNA, 2124 bp, Artificial Sequence
  CAR M4
gccaccatggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagcgagagcaagtacggccccccctgccccccctgccccggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggaatccaaatacggaccaccatgcccaccatgcccaggaggtggcggaagtgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcgaatccaaatacggaccaccatgcccaccatgcccaggcggtggcggcagcttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 78 - DNA, 1755 bp, Artificial Sequence
  CAR 2S5
gccaccatggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagcgagagcaagtacggccccccctgccccccctgccccggtggcggtggaagtgtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcgaatccaaatacggaccaccatgcccaccatgcccaggcggtggcggcagcttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 79 - DNA, 2001 bp, Artificial Sequence
  CAR M6
gccaccatggagttcggcctgagctggctgttcctggtggccatcctgaagggcgtgcagtgcagcagggacatccagatgacccagaccaccagcagcctgagcgccagcctgggcgacagggtgaccatcagctgcagggccagccaggacatcagcaagtacctgaactggtaccagcagaagcccgacggcaccgtgaagctgctgatctaccacaccagcaggctgcacagcggcgtgcccagcaggttcagcggcagcggcagcggcaccgactacagcctgaccatcagcaacctggagcaggaggacatcgccacctacttctgccagcagggcaacaccctgccctacaccttcggcggcggcaccaagctggagctgaagaggggcggtggaggttccggcggtggcggttccggaggcggtgggtcaggaggtggaggctccgaggtgcagctgcagcagagcggccccggcctggtggcccccagccagagcctgagcgtgacctgcaccgtgagcggcgtgagcctgcccgactacggcgtgagctggatcaggcagccccccaggaagggcctggagtggctgggcgtgatctggggcagcgagaccacctactacaacagcgccctgaagagcaggctgaccatcatcaaggacaacagcaagagccaggtgttcctgaagatgaacagcctgcagaccgacgacaccgccatctactactgcgccaagcactactactacggcggcagctacgccatggactactggggccagggcaccaccgtgaccgtgagcagcggtggcggtggaagtgccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcggcggtggcggcagcttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggtaa

SEQ ID NO: 80 - protein, 15 aa, Homo sapiens
EPKSPDKTHTCPPCP

SEQ ID NO: 81 - protein, 12 aa, Homo sapiens
ERKCCVECPPCP

SEQ ID NO: 82 - protein, 16 aa, Homo sapiens
ELKTPLGDTHTCPRCP

SEQ ID NO: 83 - protein, 12 aa, Homo sapiens
ESKYGPPCPPCP

SEQ ID NO: 84 - protein, 23 aa, Artificial Sequence
  linker C2
ETIRESKYGPPCPPCPGGGGSVP

(19) United States
(12) Patent Application Publication
(10) Pub. No.: US 2024/0109978 A1
(43) Pub. Date: Apr. 4, 2024

(54) CHIMERIC ANTIGEN RECEPTOR (CAR) SPACER MODIFICATIONS ENHANCE CAR T CELL FUNCTIONALITY

(71) Applicant: ORION CORPORATION, Espoo (FI)

(72) Inventors: Matti KORHONEN, Helsinki (FI); Jan KOSKI, Helsinki (FI)

(21) Appl. No.: 18/257,761

(22) PCT Filed: Dec. 14, 2021
(86) PCT No.: PCT/FI2021/050870

§ 371 Date: Jun. 15, 2023

(30) Foreign Application Priority Data
Dec. 16, 2020 (FI) 20206315

(51) Int. Cl. C07K 16/32; A61K 39/00; C07K 14/705; C07K 14/725; C12N 5/0783

(52) U.S. Cl. CPC C07K 16/32; A61K 39/4611; A61K 39/4631; A61K 39/464406; C07K 14/70503; C07K 14/7051; C07K 14/70521; C12N 5/0636; A61K 2239/17; C07K 2317/53; C07K 2317/622; C07K 2319/03; C12N 2510/00

(57) ABSTRACT

The present invention relates to chimeric antigen receptors (CAR) comprising an inert and modifiable spacer that evades the off-target binding by Fc receptor (FcR) expressing cells in CAR T cell therapy. The spacer is based on Ig-like C1 domain of signal-regulatory protein alpha.

### FIELD OF THE INVENTION

The present invention relates to chimeric antigen receptors (CAR) comprising an inert and modifiable spacer that evades the off-target binding by Fc receptor (FcR) expressing cells in CAR T cell therapy. The spacer is based on Ig-like C1 domains of signal-regulatory protein alpha.

### BACKGROUND OF THE INVENTION

Chimeric antigen receptor (CAR) based T cell therapies are a novel therapy modality for hematological cancers and have shown remarkable results in treatment of refractory and relapsed patients with acute lymphocytic leukemia (ALL), diffuse large B-cell lymphoma (DLBCL) and Non-Hodkin's lymphoma. However, in the advancing therapies current CARs need to be improved to attain highly efficient but tolerable cytotoxicity by preventing the previously identified and possible yet to be identified side-effects. Fine-tuning the CARs for evading the spacer-related interactions with off-target cells and comparing optimal spacer-modifications have not been extensively studied and need a more accurate insight for adjusting the cytotoxic responsiveness.

The spacer with its structural functions between the cell membrane and antigen binding domain has an important role in fine-tuning the CAR related antigen-independent or -dependent signaling. Commonly used CARs have spacer composed of Immunoglobulin G (IgG) constant domains, extracellular domains of CD8-alpha or CD28, extracellular moiety of NGFR (Casucci et al. 2018) or NKG2D (Sentman et al. 2014). The IgG1-CH2 domain of the Fc-region in traditional IgG1-based CARs (IgG1-CAR) interacts with FcR-expressing myeloid cells, commonly monocytes or macrophages or with NK cells, which may lead to myeloid cell activation and inflammation (Almåsbak et al 2015). The FcR binding to CARs may lead to CAR T cell activation and destruction of FcR-expressing myeloid cells, sequestration of CAR T cells in the lungs, activation induced cell death (AICD) and overall reduction of CAR T cell activity (Almåsbak et al 2015, Hombach et al 2010, Hudecek et al 2015). The unwanted interactions with off-target cells and the conceivable side effects must be avoided to achieve functional therapeutic CAR T cells.

Signal regulatory protein (SIRP) family, also known e.g. SHPS, CD172, members are membrane proteins involved in leukocyte function regulation (van Beek et al 2005). Extracellular regions of SIRP family members are typically composed of a single Ig-like V-type domain and two Ig-like C1-type domains. SIRP-alpha (also known SHPS-1, BIT, MFR, CD172a, p84) is a SIRP family member with a typical extracellular region consisting of a single Ig-like V-type domain, Ig-like C1-type 1 domain and Ig-like C1-type 2 domain (van Beek et al 2005). The extracellular region of SIRP-alpha is known extracellularly only to bind the target ligand CD47 via its V-type Ig-like domain in the N-terminus (Hatherley D et al 2009), while Ig-like C1-type domains of SIRP-alpha are currently known as an inert backbone.

### SUMMARY OF THE INVENTION

The current invention relates to a chimeric antigen receptor (CAR) comprising an extracellular spacer which comprises at least one Ig-like C1 domain of signal-regulatory protein alpha (SIRP-alpha) or its fragment or its variant.

In some embodiments Ig-like C1 domain of SIRP-alpha is selected from (i) type 1 domain according to SEQ ID NO 1 or its fragment or its variant; or (ii) type 2 domain according to SEQ ID NO 2 or its fragment or its variant.

In some embodiments the extracellular spacer comprises Ig-like C1 type 1 domain and Ig-like C1 type 2 domain of SIRP-alpha.

In some embodiments the extracellular spacer further comprises at least one multimerization domain, wherein the multimerization domain is selected or multiple multimerization domains are selected from IgG hinge regions selected from IgG1 hinge region according to SEQ ID NO 4 or SEQ ID NO 80, IgG2 hinge region according to SEQ ID NO 81, IgG3 hinge region according to SEQ ID NO 82, IgG4 hinge region according to SEQ ID NO 83 and/or extracellular domain of CD28 according to SEQ ID NO 3 and/or their fragments and variants. In some embodiments the multimerization domain is selected or multiple multimerization domains are selected from IgG1 hinge region according to SEQ ID NO 4 or its fragment and/or extracellular domain of CD28 according to SEQ ID NO 3 or its fragment. In some embodiments the multimerization domain is selected or multiple multimerization domains are selected from IgG4 hinge region according to SEQ ID NO 83 or its fragment and/or extracellular domain of CD28 according to SEQ ID NO 3 or its fragment.

In some embodiments the extracellular spacer locates between a transmembrane domain and an antigen binding domain. In some embodiments the antigen binding domain is a single chain variable region (scFv)

In some embodiments the extracellular spacer dimerizes CAR at least with one disulfide bridge. Extracellular CD28 comprises one disulfide bridge. IgG hinge region comprises two disulfide bridges. In some embodiments the CAR dimerizes with one disulfide bridge, two disulfide bridges or three disulfide bridges.

The current invention also relates to CAR comprising an extracellular spacer comprising amino acid sequence according to SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 56, SEQ ID NO 57, SEQ ID NO 58, SEQ ID NO 59, SEQ ID NO 60 or SEQ ID NO 61.

In some embodiments the CAR comprises any previous extracellular spacer domain, an antigen binding domain, a transmembrane domain, an intracellular signaling domain, and optionally a costimulatory domain.

In some embodiments the antigen binding domain of a CAR comprises an antibody or its fragment.

In some embodiments the antigen binding domain of a CAR comprises a single chain variable fragment (scFv).

In some embodiment the antigen binding domain of a CAR targets a tumor antigen or cancer antigen. The tumor antigen may be selected from CD19, HER-2, BCMA, CD22, CS1, CD38, CD33, CD20, CD30, CD38, CD123, TAA, GD2, MSLN, EGFR, EBV, GPC3, MUC1, PSMA, NY-ESO-1 reviewed in Yu et al 2020 and Townsend et al 2018. The tumor antigen targeted by the CARs of the current invention is preferably selected from CD19 or HER-2.

In some embodiments the transmembrane domain of a CAR is selected from transmembrane domain of a membrane protein. The transmembrane domain may be selected from CD28, CD8, CD8alpha, OX40L receptor (also known as CD134), 4-1BB (also known as CD137), CD3, CD3delta, CD3gamma, CD3epsilon or CD3zeta or their fragments. In a preferred embodiment the transmembrane domain of a CAR comprises transmembrane domain of CD28 according to SEQ ID NO 23 or its fragment.

An intracellular signaling domain of a CAR may be selected from intracellular domain of CD3zeta, CD3delta, CD3gamma, CD3epsilon, CD28, FcgammaRIII, FcR cytoplasmic tail or tyrosine kinases or their fragments. In preferred embodiments the intracellular signaling domain comprises intracellular domain of CD3zeta according to SEQ ID NO 25 or its fragments.

A co-stimulatory domains of CAR may be selected from CD28, CD8, CD8alpha, OX4OL receptor (also known as CD134), 4-1BB (also known as CD137), KIR2DS2, ICOS, CD27, MYD88-D40 or their fragments or their variants. The co-stimulatory domain of a CAR preferably comprises intracellular CD28 according to SEQ ID NO 24 or its fragment.

The current invention also relates to a chimeric antigen receptor (CAR) comprising
i. a single chain variable fragment (scFv);
ii. IgG hinge domain;
iii. Ig-like C1 type 1 and/or Ig-like C1 type 2 domain of signal-regulatory protein alpha-1;
iv. CD3zeta;
v. CD28 transmembrane domain;
vi. optionally CD28 extracellular domain and/or CD28 intracellular domain.

The current invention also relates a CAR comprising or consisting an amino acid sequence according to SEQ ID NO 26, SEQ ID NO 27, SEQ ID NO 28, SEQ ID NO 29 SEQ ID NO 30, SEQ ID NO 31, SEQ ID NO 32, SEQ ID NO 33, SEQ ID NO 34, SEQ ID NO 54, SEQ ID NO 62, SEQ ID NO 63, SEQ ID NO 64, SEQ ID NO 65, SEQ ID NO 66 or SEQ ID NO 67.

The current invention further relates to a polynucleotide encoding any of the previously described CARs.

The current invention also relates to a vector comprising a polynucleotide encoding any of the previously described CARs.

The current invention also relates to a cell comprising any of the previously described CARs or any of the polynucleotides encoding them. In some embodiment the cell is a T-cell.

The invention further relates to a method to adjust the length of a CAR by selecting at least two domains from (i) IgG hinge domain, (ii) Ig-like C1 type 1 domain of signal-regulatory protein alpha-1, (iii) Ig-like C1 type 2 domain of signal-regulatory protein alpha-1 or (iv) CD28 extracellular fragment to the spacer domain resulting in chimeric antigen receptors with different lengths.

In some embodiments the extracellular spacer domain does not bind or has reduced binding affinity to Fc receptor.

### DESCRIPTION OF THE DRAWINGS

FIG. 1 Schematic figure of spacer modified CARs and T cell expansion kinetics (n=3). A) CAR domains and designed structures in a schematic model. CAR 1S and CAR X1S are not present in the figure. CAR 1S and CAR X1S correspond CAR 2S and CAR X2S, respectively, except that SIRP-alpha Ig-like C1 type 2 domain is SIRP-alpha Ig-like C1 type 1 domain. B) T cell viability was assessed with trypan blue and counted with Bio-Rad TC20 Automated Cell Counter on days 2, 3, 6, 8 and 10 prior subculturing the cells. Results are shown as mean values with standard deviation. C) Subculturing based fold expansion was counted every 2-3 days and evaluated for fold expansion between subcultures. Lines represent mean values (with SD) of the different CARs. D) CAR expression on day 13 was analyzed by flow cytometry. Results show individual data points and mean values (lines).

FIG. 2 Cell phenotypes after expansion. T cell products (n=3) were expanded for 13 days and their phenotypes analyzed by flow cytometry. Results are shown as individual data points with mean values. A) Cell phenotypes were determined with the following antibody combinations: T cells CD3+CD56−; NKT cells CD3+CD56+; NK cells CD3-CD56+ and other cells CD3-CD56−. B) and C) The proportions of CD4 and CD8 positive cells in T cell and NKT cell populations.

FIG. 3 Percentages of different memory phenotypes, exhausted and terminally differentiated T and NKT cells. Results (measured by flow cytometry) indicate mean values with minimum and maximum values (FIG. A) or individual data points with mean value (FIGS. B and C). A) On day 13 of the expansion, cells were analyzed for memory phenotypes. B) SCM, SCM-like and CM memory phenotypes were grouped together as an ‘early memory phenotype’ group and EM and Eff as an ‘effector phenotype’ group. C) The cells were analyzed for the exhausted (PD-1 positive) and terminally differentiated (CD57 positive) groups.

FIG. 4 T cell responses and cytotoxicity against CD19 positive Nalm-6 cells. The mean (black horizontal lines) and individual data points are shown (FIGS. A and B) A) CART cells were cocultured with Nalm-6 cells at 1:1 E:T ratio for 18 h. Cytokines were analyzed from coculture supernatants using a flow cytometry-based CBA array. B) Degranulation of T cells in response to CD19 positive Nalm-6 cells was analyzed by staining the CD107a in T cells after 4 h coculture in the presence on GolgiStop protein transport inhibitor. The results indicate %-value of CD107a expressing cells in T cells and from those values the percentage of CD4 and CD8 positive cells. C) Luciferase activity was measured to analyze in vitro cytotoxicity of CAR T cells against luciferase-expressing CD19+ Nalm-6 cells at various E:T ratios. The mean+/−SD is shown.

FIG. 5 CAR T cell interactions with FcR-expressing THP-1 monocytes. CAR T cells were cocultured with monocytes at a 1:1 (effector cell:off-target cell) ratio. The activation of CAR T cells was measured by staining the cell surface activation markers (FIG. 5A: CD25, CD69; flow cytometry) and by measuring the CAR T cell and monocyte activation induced cytokines using a flow cytometry-based CBA array (FIG. 5B: CAR T cells: IFN-gamma and IL-2; FIG. 5C: monocytes: IL-1beta).

FIG. 6 CAR expression and cytotoxic efficacy of Jurkat T cells encoding CARs with various lengths. A) CAR expression was measured by flow cytometry after transduction (mock, CAR 2S and IgG CAR) or after transduction and positive selection (CAR M, CAR XM, CAR L, and CAR XL). Results are shown in contour plots. B) In vitro cytotoxicity was assessed by measuring the luciferase activity of CD19 Nalm-6-luc cells at various E:T ratios. The results are presented as mean values +/−SD (n=3).

FIG. 7 Cytotoxicity of CAR with HER-2 targeting antigen binding domain CAR M against HER-2 positive SKBR-3 breast carcinoma cells. Luciferase activity was measured to quantify the in vitro cytotoxicity of HER-2 targeting CAR T cells against luciferase expressing HER-2 positive SKBR-3 cells at various E:T ratios. The results show mean value +/−SD.

FIG. 8 Cytotoxicity of T cells expressing HER-2 targeting CAR M against HER-2 positive SKBR-3 breast carcinoma cells. Luciferase activity was measured to quantify the in vitro cytotoxicity of HER-2 targeting CAR T cells against luciferase expressing HER-2 positive SKBR-3 cells at various E:T ratios.

FIG. 9 Cell expansion, CAR expression and cytotoxicity of CAR constructs with modified multimerization domains. A) Expansion of T cells from the same donor transduced with lentiviruses with CAR constructs CAR M, CAR XM, CAR M1, CAR XM2, CAR XM3, CAR M4, CAR2S5 and CAR M6. Expansion fold is relative to the number of T-cells at the start of the experiment. Expansion fold was measured on day 1, 3, 6, 8 and 10. B) Chimeric antigen receptor expression of the CAR constructs. Chimeric antigen receptor expression of the T cells was detected from the surface of the cells with an antibody. The vector copy number was measured with quantitative PCR from isolated genomic DNA. Percent of viable cells and vector copy number is shown for CAR constructs CAR M, CAR XM, CAR M1, CAR XM2, CAR XM3, CAR M4, CAR2S5 and CAR M6. C) Cytotoxicity of CAR-T effector cells (CAR constructs CAR M, CAR XM, CAR M1, CAR XM2, CAR XM3, CAR M4, CAR2S5 and CAR M6) were co-cultured with NALM-6 target cells at different ratios for 24 hours. Effector-target (E:T) ratios 4:1, 2:1, 1:1, 0,5:1, 0,25:1, 0,125:1 and 0,0625:1 were used. Target specific transgene (luciferase) amount was measured and killing percentage relative to target cells only was determined.

### DETAILED DESCRIPTION OF THE INVENTION

Features and embodiments of the current invention are described by way of non-limiting examples in the disclosure. The present disclosure should not be considered as limitation to particular compounds, compositions, methods, uses described in the disclosure. It should be understood that a skilled person may make apparent modifications and variations to the current invention and embodiments. Singular forms a, an, the used in the application refers one or more.

To practice the current invention and embodiments the skilled person may employ common techniques and methods of biology, molecular biology, microbiology, chemistry, biochemistry, immunology and oncology. Common techniques and methods are described in literature, for example in laboratory manuals and laboratory protocols. Such literature is for example Current Protocols in Cell Biology, Current Protocols in Immunology, Current Protocols in Molecular Biology, Current Protocols in Microbiology, Molecular cloning: A Laboratory Manual. The used technical and scientific terms have the meaning commonly understood by a skilled person based on scientific literature and technology dictionaries.

Chimeric antigen receptor (CAR) or (CARs) refers to receptor protein binding to a specific antigen and participating in cell activation. CARs comprise an antigen binding domain, a spacer domain, a transmembrane domain, an intracellular signaling domain and an optionally a co-stimulatory domain. Cells expressing CAR are able to bind a specific antigen resulting to activation of the cells. CAR cells are preferably T cells, naïve T cells, memory T cells, effector T cells.

The spacer domain is an extracellular domain of a CAR. It is located between the transmembrane domain and the antigen binding domain and connects them. The spacer domain has a role in fine-tuning the signaling of the CAR.

Immunoglobulin (Ig) based spacer domain is derived from an immunoglobulin Fc region or includes fragments from immunoglobulin Fc region. The immunoglobulin Fc region may be derived from IgG, IgM, IgA or IgE. Fc region of IgG may be derived from IgG1, IgG2, IgG3 or IgG4. The IgG based spacer domain comprises CH2 and CH3 domains from IgG Fc region. An IgG based spacer domain having IgG constant regions CH2 and CH3 is described for example in Hombach et al. 2010.

Signal regulatory protein (SIRP) family, also known e.g. SHPS, CD172, members are membrane proteins involved in leukocyte function regulation (van Beek et al 2005). Extracellular regions of SIRP family members are typically composed of a single Ig-like V-type domain and two Ig-like C1-type domains. SIRP-alpha (also known SHPS-1, BIT, MFR, CD172a, p84) is a SIRP family member with a typical extracellular region having a single Ig-like V-type domain, Ig-like C1-type 1 domain and Ig-like C1-type 2 25 domain (van Beek et al 2005). The extracellular region of SIRP-alpha is known extracellularly only to bind the target ligand CD47 via its V-type Ig-like domain in the N-terminus (Hatherley D et al 2009), while the Ig-like C1-type domains of SIRP-alpha are currently known as an inert backbone. Ig-like domains typically have dimensions of about 4×2.5×2.5 nm. The amino acid sequence of SIRP-alpha is present in UniProt database with accession number P78324.

### Extracellular Spacer Domain

The spacer domain of the current invention comprises at least one Ig-like C1 domain of signal regulatory protein alpha (SIRP-alpha). Signal regulatory protein alpha is abbreviated SIRP-alpha throughout the application. SIRP-alpha Ig-like C1 domain is selected from type 1 domain (SEQ ID NO 1) and/or type 2 domain (SEQ ID NO 2). In one embodiment a spacer comprises SIRP-alpha Ig-like C1-type 1 domain. In another embodiment a spacer comprises SIRP-alpha Ig-like C1-type 2 domain. In another embodiment a spacer comprises SIRP-alpha Ig-like C1-type 1 domain and SIRP-alpha Ig-like C1-type 2 domain. The spacer may comprise multiple SIRP-alpha Ig-like C1-type 1 domains and/or SIRP-alpha Ig-like C1-type 2 domains.

The spacer may comprise a multimerization domain. A multimerization domain multimerizes the CAR monomers. In multimerization CARs may form dimers, trimers, quadramers, pentamers or multimers from CAR monomers. Preferably the CARs form dimers formed from two CAR monomers. Multimerization domain is capable to form linkages between monomers of CARs. Preferably the linkages between the monomers are disulfide bridges. Preferably the multimerization domain forms at least one, two, or three disulfide bridges between the monomers. In some embodiments of the invention the multimerization domain of the spacer is selected from group: IgG1 hinge region, IgG2 hinge region, IgG3 hinge region, IgG4 hinge region, extracellular CD28 domain or their fragments or variants. In some embodiments the spacer comprises the multimerization domain comprising IgG1 hinge region or its fragments. In some embodiments the spacer comprises the multimerization domain comprising IgG4 hinge region or its fragments. In a preferred embodiment the multimerization domain comprises amino acid sequence according to SEQ ID NO 4. In a preferred embodiment the multimerization domain comprises amino acid sequence according to SEQ ID NO 80 or SEQ ID NO 83. The IgG1 hinge region or its fragment is combined from one end to SIRP-alpha Ig-like C1 type domain and from the other end to antigen binding domain of CAR. The IgG4 hinge region or its fragment is combined from one end to SIRP-alpha Ig-like C1 type domain and from the other end to antigen binding domain of CAR. An additional linker sequence may be used for combination. In another embodiment the spacer comprises the multimerization domain comprising extracellular CD28 domain or its fragments. In a preferred embodiment the multimerization domain comprises amino acid sequence according to SEQ ID NO 3.

The extracellular CD28 domain or its fragment is combined from one end to SIRP-alpha IG-like C1 type domain and from the other end to the transmembrane domain, for example to transmembrane domain of CD28 (SEQ ID NO 23). An additional linker sequence may be used for combination. The spacer may comprise multiple multimerization domains. The spacer may comprise multiple different multimerization domains. In some embodiments the spacer comprises both IgG1 hinge region and extracellular CD28 domain. In some embodiments the spacer comprises both IgG4 hinge region and extracellular CD28 domain.

The spacer domain locates between the transmembrane domain and the antigen binding domain and connects them. The spacer domain has a role in fine-tuning antigen signaling of the CAR. In current invention the length of the spacer is adjustable by using different domains and their combinations in the spacer. It results in different spacer lengths and optimal binding of CAR to its antigen. In some embodiments the domains in the spacer may be selected from Ig-like C1 type 1 domain of SIRP-alpha, Ig-like C1 type 2 domain of SIRP-alpha, extracellular CD28 domain and/or IgG hinge region and or their fragments or variants. Table 1 presents amino acid sequences of different CAR spacers comprising selected domains resulting to different lengths of the spacers (SEQ ID NOs 10-18, 56-61).

In immunoglobulin (Ig) based CARs, CH2 domain interacts with the Fc receptor (FcR) of myeloid cells. Myeloid cells expressing FcR are for example monocytes, macrophages, and NK cells. The FcR binding to CAR may lead to CAR T cell activation and destruction of FcR-expressing myeloid cells, sequestration of CAR T cells in the lungs, activation induced cell death (AICD) and overall reduction of CAR T cell activity (Almåsbak et al 2015, Hombach et al 2010, Hudecek et al 2015). The unwanted interactions with off-target cells and the conceivable side effects must be avoided to achieve functional therapeutic CAR T cells.

In current invention the spacer domain comprises at least one Ig-like C1 domain of signal-regulatory protein alpha or its fragment. The Ig-like C1 domain is selected from type 1 domain and/or type 2 domain. Preferably the spacer comprises Ig-like C1 type 1 domain and Ig-like C1 type 2 domain. The spacer domains of the current invention do not interact with FcR of myeloid cells resulting in functional effects. T cells with CAR of the current invention do not effect CAR T cell activation caused by off-target binding, destruction of FcR-expressing myeloid cells, sequestration of CAR T cells in the lungs, activation induced cell death (AICD) and overall reduction of CAR T cell activity.

In the preferred embodiments of the invention the spacer domain comprises amino acid sequence of SEQ ID NO 10, SEQ ID NO 11, SEQ ID NO 12, SEQ ID NO 13, SEQ ID NO 14, SEQ ID NO 15, SEQ ID NO 16, SEQ ID NO 17 or SEQ ID NO 18 or their variants or fragments. Their variants have at least 80%, 85%, 90%, 95%, 96%, 97%, 98% , 99% sequence identity to any of SEQ ID NOs 10-18. Amino acid sequences of the spacer domains are summarized in table 1.

In the preferred embodiments of the invention the spacer domain comprises amino acid sequence of SEQ ID NO 56, SEQ ID NO 57, SEQ ID NO 58, SEQ ID NO 59, SEQ ID NO 60, or SEQ ID NO 61 or their variants or fragments. Their variants have at least 80%, 85%, 90%, 95%, 96%, 97%, 98%, 99% sequence identity to any of SEQ ID NOs 56-61. Amino acid sequences of the spacer domains are summarized in table 1.

CAR spacer XS according to SEQ ID NO 10 comprises IgG1 hinge region and CD28 extracellular fragment.

CAR spacer 1S according to SEQ ID NO 11 comprises IgG1 hinge region and SIRP-alpha Ig-like C1 type 1 domain.

CAR spacer 2S according to SEQ ID NO 12 comprises IgG1 hinge region and SIRP-alpha Ig-like C1 type 2 domain.

CAR spacer X1S according to SEQ ID NO 13 comprises IgG1 hinge region, SIRP-alpha Ig-like C1 type 1 domain and CD28 extracellular fragment.

CAR spacer X2S according to SEQ ID NO 14 comprises IgG1 hinge region, SIRP-alpha Ig-like C1 type 2 domain and CD28 extracellular fragment.

CAR spacer M according to SEQ ID NO 15 comprises IgG1 hinge region, SIRP-alpha Ig-like C1 type 1 domain and SIRP-alpha Ig-like C1 type 2 domain.

CAR spacer XM according to SEQ ID NO 16 comprises IgG1 hinge region, SIRP-alpha Ig-like C1 type 1 domain, SIRP-alpha Ig-like C1 type 2 domain and CD28 extracellular fragment.

CAR spacer L according to SEQ ID NO 17 comprises IgG1 hinge region, SIRP-alpha Ig-like C1 type 2 domain, SIRP-alpha Ig-like C1 type 1 domain and SIRP-alpha Ig-like C1 type 2 domain.

CAR spacer XL according to SEQ ID NO 18 comprises IgG1 hinge region, SIRP-alpha Ig-like C1 type 2 domain, SIRP-alpha Ig-like C1 type 1 domain and SIRP-alpha Ig-like C1 type 2 domain and CD28 extracellular fragment.

CAR spacer M1 according to SEQ ID NO 56 comprises IgG4 hinge region, SIRP-alpha Ig-like C1 type 1 domain and SIRP-alpha Ig-like C1 type 2 domain.

CAR spacer XM2 according to SEQ ID NO 57 comprises IgG4 hinge region, SIRP-alpha Ig-like C1 type 1 domain, SIRP-alpha Ig-like C1 type 2 domain and CD28 extracellular fragment.

CAR spacer XM3 according to SEQ ID NO 58 comprises IgG4 hinge region, SIRP-alpha Ig-like C1 type 1 domain, IgG4 hinge region, SIRP-alpha Ig-like C1 type 2 domain and CD28 extracellular fragment.

CAR spacer M4 according to SEQ ID NO 59 comprises IgG4 hinge region, SIRP-alpha Ig-like C1 type 1 domain, IgG4 hinge region, SIRP-alpha Ig-like C1 type 2 domain and IgG4 hinge region.

CAR spacer 2S5 according to SEQ ID NO 60 comprises IgG4 hinge region, SIRP-alpha Ig-like C1 type 2 domain and IgG4 hinge region.

CAR spacer M6 according to SEQ ID NO 61 comprises SIRP-alpha Ig-like C1 type 1 domain and SIRP-alpha Ig-like C1 type 2 domain.

All the above CAR spacers may comprise linker sequences combining the domains to each other. All the CAR spacers and their amino acid sequences are summarized in table 1.

### Antigen Binding Domain

The antigen binding domain of chimeric antigen receptor recognizes an antigen. The antigen binding domain of a CAR binds to an epitope of said antigen. Antigen binding domain may comprise a protein, a peptide, or their mimetics binding to the antigen. In some embodiment the antigen binding domain is an antibody or its functional fragment. Antibody refers to an immunoglobulin specifically binding to an epitope of an antigen. The antibody may be monoclonal antibody or polyclonal antibody. Antibody or its functional fragments include without limitation chimeric antibodies, humanized antibodies, bispecific antibodies, nanobodies, camelid antibodies, fragment antigen-binding (Fab), bivalent Fab region (F(ab′)2), single chain antibody fragment (scAb) Fv, single chain variable fragment (scFv), bivalent scFv (sc(Fv)2). In some embodiment the antigen binding domain comprises a single chain variable fragment (scFv). The scFv comprises variable light chain variable (VL) and variable heavy chain (VH).

Various antigens are known to be associated with cancer. The cancer associated antigen may be an antigen expressed by a cancer cell. The cancer associated antigen may be overexpressed by a cancer cell. The cancer associated antigen may be a mutated product of a gene, or product of a normal gene that is expressed on a cancer cell in a such quantity that it can be targeted using CARs. The cancer associated antigen may be protein, peptide, carbohydrate, glycoprotein, glycolipid, proteoglycan, proteolipids or any of their combinations. Some cancer associated antigens are reviewed by Townsend et al 2018, Yu et al 2020.

In some embodiments the antigen binding domain of CAR binds to a cancer associated antigen. Cancer associated antigen may be selected for example from known cancer associated antigens. Such antigens are reviewed by Townsend et al 2018, Yu et al 2020. In some embodiments the antigen binding domain binds to CD19. In some embodiments the antigen binding domain binding to CD19 is a single chain variable fragment (scFv). In some embodiment the antigen binding domain binding to CD19 is an scFV comprising SEQ ID NO 22 or its variant having at least 80%, 85%, 90%, 95%, 96%, 97%, 98%, 99% sequence identity to SEQ ID NO 22. In some embodiments the antigen binding domain binds to HER-2. In some embodiments the antigen binding domain binding to HER-2 is a single chain variable fragment (scFv). In some embodiment the antigen binding domain binding to HER-2 is an scFV comprising SEQ ID NO 53 or its variant having at least 80%, 85%, 90%, 95%, 96%, 97%, 98%, 99% sequence identity to SEQ ID NO 53.

### Transmembrane Domain

Transmembrane domain of a CAR may be selected or derived from any transmembrane domain of membrane proteins. Transmembrane domain of a CAR may be for example transmembrane domain of CD28, CD8, CD8alpha, OX4OL receptor (also known as CD134), 4-1BB (also known as CD137), CD3, CD3delta, CD3gamma,

CD3epsilon, CD3zeta. In some embodiments the transmembrane domain of a CAR is transmembrane domain of CD28 or its fragment or its variant. In some embodiments the transmembrane domain of the CAR comprises amino acid sequence according to SEQ ID NO 23.

### Signaling Domain

A CAR may comprise an intracellular signaling domain. Intracellular signaling domain may be cytoplasmic. The intracellular signaling domain of a CAR mediates the signal resulting in effector function in a cell expressing the CAR. The intracellular signaling domain of the CAR may for example mediate CAR signal to T cell activation. The intracellular signaling domain may be selected from CD3zeta, CD3delta, CD3gamma, CD3epsilon, CD28, FcgammaRIII, FcR cytoplasmic tail, intracellular domains of tyrosine kinases. In some embodiments the intracellular signaling domain comprises intracellular domain of CD3zeta or its fragments. In some embodiments the intracellular signaling domain comprises amino acid sequence according to SEQ ID NO 25 or its fragment.

### Co-stimulatory Domain

A CAR may comprise optionally one or more co-stimulatory domains. Co-stimulatory domain is cytoplasmic and may influence on cell proliferation, phenotype differentiation. Co-stimulatory domains of the CAR may be selected for example from CD28, CD8, CD8alpha, OX4OL receptor (also known as CD134), 4-1BB (also known as CD137), KIR2DS2, ICOS, CD27, MYD88-D40 or their fragments or their variants. In some embodiments the co-stimulatory domain of the CAR comprises intracellular CD28 or its fragment or its variant. In some embodiment the co-stimulatory domain of the CAR comprises amino acid sequence according to SEQ ID NO 24.

In some embodiments the intracellular or cytoplasmic region of a CAR comprises an intracellular signaling domain and a co-stimulatory domain. In some embodiments the intracellular region of the CAR comprises CD3zeta or its fragment and intracellular CD28 domain or its fragment. In some embodiments the cytoplasmic region of the CAR comprises amino acid sequence according to SEQ ID NO 24 or its fragment and amino acid sequence according to SEQ ID NO 25 or its fragment.

### CARs

CARs comprise an antigen binding domain, a spacer domain, a transmembrane domain, an intracellular signaling domain and an optionally a co-stimulatory domain. CARs of the current invention may be selected from amino acid sequences according to SEQ ID NO 26, SEQ ID NO 27, SEQ ID NO 28, SEQ ID NO 28, SEQ ID NO 29, SEQ ID NO 30 30, SEQ ID NO 31, SEQ ID NO 32, SEQ ID NO 33, SEQ ID NO 34 or SEQ ID NO 54 or their variants or their fragments. Their variants have at least 80%, 85%, 90%, 95%, 96%, 97%, 98% , 99% sequence identity to any of SEQ ID NOs 26-34 or SEQ ID NO 54. The CAR structures and amino acid sequences are summarized in Table 1.

CARs of the current invention may be selected from amino acid sequences according to SEQ ID NO 62, SEQ ID NO 63, SEQ ID NO 64, SEQ ID NO 65, SEQ ID NO 66, or SEQ ID NO 67 or their variants or their fragments. Their variants have at least 80%, 85%, 90%, 95%, 96%, 97%, 98%, 99% sequence identity to any of SEQ ID NOs 62-67. The CAR structures and amino acid sequences are summarized in Table 1.

CAR XS according to SEQ ID NO 26 comprises scFv binding to CD19 as an antigen binding domain, IgG1 hinge region and CD28 extracellular fragment as a spacer domain, CD28 fragment as a transmembrane domain, CD28 intracellular fragment as a co-stimulatory domain and CD3zeta fragment as an intracellular signaling domain.

CAR 1S according to SEQ ID NO 27 comprises scFv binding to CD19 as an antigen binding domain, IgG1 hinge region and SIRP-alpha Ig-like C1 type 1 domain as a spacer domain, CD28 fragment as a transmembrane domain, CD28 intracellular fragment as a co-stimulatory domain and CD3zeta fragment as an intracellular signaling domain.

CAR 2S according to SEQ ID NO 28 comprises scFv binding to CD19 as an antigen binding domain, IgG1 hinge region and SIRP-alpha Ig-like C1 type 2 domain as a spacer domain, CD28 fragment as a transmembrane domain, CD28 intracellular fragment as a co-stimulatory domain and CD3zeta fragment as an intracellular signaling domain.

CAR X1S according to SEQ ID NO 29 comprises scFv binding to CD19 as an antigen binding domain, IgG1 hinge region, SIRP-alpha Ig-like C1 type 1 domain and CD28 extracellular fragment as a spacer domain, CD28 fragment as a transmembrane domain, CD28 intracellular fragment as a co-stimulatory domain and CD3zeta fragment as an intracellular signaling domain.

CAR X2S according to SEQ ID NO 30 comprises scFv binding to CD19 as an antigen binding domain, IgG1 hinge region, SIRP-alpha Ig-like C1 type 2 domain and CD28 extracellular fragment as a spacer domain, CD28 fragment as a transmembrane domain, CD28 intracellular fragment as a co-stimulatory domain and CD3zeta fragment as an intracellular signaling domain.

CAR M according to SEQ ID NO 31 comprises scFv binding to CD19 as an antigen binding domain, IgG1 hinge region, SIRP-alpha Ig-like C1 type 1 domain and SIRP-alpha Ig-like C1 type 2 domain as a spacer domain, CD28 fragment as a transmembrane domain, CD28 intracellular fragment as a co-stimulatory domain and CD3zeta fragment as an intracellular signaling domain.

CAR XM according to SEQ ID NO 32 comprises scFv binding to CD19 as an antigen binding domain, IgG1 hinge region, SIRP-alpha Ig-like C1 type 1 domain, SIRP-alpha Ig-like C1 type 2 domain and CD28 extracellular fragment as a spacer domain, CD28 fragment as a transmembrane domain, CD28 intracellular fragment as a co-stimulatory domain and CD3zeta fragment as an intracellular signaling domain.

CAR L according to SEQ ID NO 33 comprises scFv binding to CD19 as an antigen binding domain, IgG1 hinge region, SIRP-alpha Ig-like C1 type 2 domain, SIRP-alpha Ig-like C1 type 1 domain and SIRP-alpha Ig-like C1 type 2 domain as a spacer domain, CD28 fragment as a transmembrane domain, CD28 intracellular fragment as a co-stimulatory domain and CD3zeta fragment as an intracellular signaling domain.

CAR XL according to SEQ ID NO 34 comprises scFv binding to CD19 as an antigen binding domain, IgG1 hinge region, SIRP-alpha Ig-like C1 type 2 domain, SIRP-alpha Ig-like C1 type 1 domain and SIRP-alpha Ig-like C1 type 2 domain and CD28 extracellular fragment as a spacer fragment, CD28 fragment as a transmembrane domain, CD28 intracellular fragment as a co-stimulatory domain and CD3zeta fragment as an intracellular signaling domain.

HER-2 CAR M according to SEQ ID NO 54 comprises scFv binding to HER-2 as an antigen binding domain, IgG1 hinge region, SIRP-alpha Ig-like C1 type 1 domain and SIRP-alpha Ig-like C1 type 2 domain as a spacer domain, CD28 fragment as a transmembrane domain, CD28 intracellular fragment as a co-stimulatory domain and CD3zeta fragment as an intracellular signaling domain.

CAR M1 according to SEQ ID NO 62 comprises scFv binding to CD19 as an antigen binding domain, IgG4 hinge region, SIRP-alpha Ig-like C1 type 1 domain and SIRP-alpha Ig-like C1 type 2 domain as a spacer domain, CD28 fragment as a transmembrane domain, CD28 intracellular fragment as a co-stimulatory domain and CD3zeta fragment as an intracellular signaling domain.

CAR XM2 according to SEQ ID NO 63 comprises scFv binding to CD19 as an antigen binding domain, IgG4 hinge region, SIRP-alpha Ig-like C1 type 1 domain, SIRP-alpha Ig-like C1 type 2 domain and CD28 extracellular fragment as a spacer domain, CD28 fragment as a transmembrane domain, CD28 intracellular fragment as a co-stimulatory domain and CD3zeta fragment as an intracellular signaling domain.

CAR XM3 according to SEQ ID NO 64 comprises scFv binding to CD19 as an antigen binding domain, IgG4 hinge region, SIRP-alpha Ig-like C1 type 1 domain, IgG4 hinge region, SIRP-alpha Ig-like C1 type 2 domain and CD28 extracellular fragment as a spacer domain, CD28 fragment as a transmembrane domain, CD28 intracellular fragment as a co-stimulatory domain and CD3zeta fragment as an intracellular signaling domain.

CAR M4 according to SEQ ID NO 65 comprises scFv binding to CD19 as an antigen binding domain, IgG4 hinge region, SIRP-alpha Ig-like C1 type 1 domain, IgG4 hinge region, SIRP-alpha Ig-like C1 type 2 domain and IgG4 hinge region as a spacer domain, CD28 fragment as a transmembrane domain, CD28 intracellular fragment as a co-stimulatory domain and CD3zeta fragment as an intracellular signaling domain.

CAR 2S5 according to SEQ ID NO 66 comprises scFv binding to CD19 as an antigen binding domain, IgG4 hinge region, SIRP-alpha Ig-like C1 type 2 domain and IgG4 hinge region as a spacer domain, CD28 fragment as a transmembrane domain, CD28 intracellular fragment as a co-stimulatory domain and CD3zeta fragment as an intracellular signaling domain.

CAR M6 according to SEQ ID NO 67 comprises scFv binding to CD19 as an antigen binding domain, SIRP-alpha Ig-like C1 type 1 domain and SIRP-alpha Ig-like C1 type 2 domain as a spacer domain, CD28 fragment as a transmembrane domain, CD28 intracellular fragment as a co-stimulatory domain and CD3zeta fragment as an intracellular signaling domain.

All the above CARs may comprise linker sequences combining the domains to each other. All the CARs and their amino acid sequences are summarized in table 1.

CARs of the current invention have a signal-regulatory protein alpha (SIRP-alpha) based backbone to provide an inert and modifiable universal spacer for CAR T cell and in other cellular therapies that evades the off-target binding to Fc receptor (FcR) expressing cells. Off-target binding via FcR with myeloid cells leads to hampered CAR T cell function, redundant cytokine production and overall impairment CAR T cells.

All the novel CARs with SIRP-alpha backbones had minor changes in CD4:CD8 ratio favoring CD4+ population, nevertheless, had equal cytotoxicity and functionality compared to the traditional IgG-based CAR.

T cells carrying SIRP-alpha based CARs showed no increased activation levels after co-culture with THP-1 monocytes in contrast to T cells with hlgG-CH2CH3 based CAR that expressed high levels of the early activation marker CD69 and IL-2 and IFN-gamma. Monocyte activation, measured by production of IL-1beta, was also avoided in SIRP-alpha CAR T-cells, in contrast to T cells with the IgG based CAR.

### Polynucleotides and Vectors

The current invention relates to polynucleotides encoding the chimeric antigen receptors of the invention. The polynucleotides may be DNA or RNA or modified DNA or modified RNA or nucleic acid analogues. The polynucleotides may be single-stranded or double-stranded. The polynucleotides of the current invention may be isolated, purified, recombinantly produced or synthesized by any methods available to a skilled person. Nucleosides of the polynucleotides may be chemically modified. Nucleic acid analogues are structurally similar compounds as DNA and RNA. Nucleic acid analogues may be for example peptide nucleic acids (PNA), locked nucleic acids (LNA), bridged nucleic acids (BNA), morpholino. Polynucleotides may comprise one or more nucleoside analogues.

It should be also understood that similar amino acid sequences may be encoded by alternative polynucleotide sequences. Codon optimization in this invention was performed using Homo sapiens codons by means of estimated probabilities based on frequency distribution in endogenous receptors. In some embodiments of the current invention the polynucleotide sequences encoding a CAR spacer may be selected from SEQ ID NO 35, SEQ ID NO 36, SEQ ID NO 37, SEQ ID NO 38, SEQ ID NO 39, SEQ ID 40, SEQ ID NO 41, SEQ ID NO 42 or SEQ ID NO 43. In some embodiments of the current invention the polynucleotide sequences encoding a CAR spacer may be selected from SEQ ID NO 68, SEQ ID NO 69, SEQ ID NO 70, SEQ ID NO 71, SEQ ID NO 72 or SEQ ID 73. In some embodiments of the current invention the polynucleotide sequences encoding a CAR may be selected from SEQ ID NO 44, SEQ ID NO 45, SEQ ID NO 46, SEQ ID NO 47, SEQ ID NO 48, SEQ ID 49, SEQ ID NO 50, SEQ ID NO 51, SEQ ID NO 52 or SEQ ID NO 55. In some embodiments of the current invention the polynucleotide sequences encoding a CAR may be selected from SEQ ID NO 74, SEQ ID NO 75, SEQ ID NO 76, SEQ ID NO 77, SEQ ID NO 78 or SEQ ID 79.

Polynucleotides encoding CARs of the current invention may form an expression cassette. Said expression cassette contains genetic information to encode a CAR of current invention. The expression cassette comprises a polynucleotide sequence encoding a CAR of the current invention. Said expression cassette may comprise coding sequences of an antigen-binding domain, a spacer domain, a transmembrane domain, an intracellular cell signaling domain, and optionally co-stimulatory domain. In addition to the coding sequences said expression cassette may comprise sequences selected from: Promoter sequences, enhancer sequences, translation stop sequences and transcription termination sequences. An expression cassette encoding the CAR of the current invention may be introduced into host cells with viral or non-viral methods.

In non-viral methods the CAR encoding polynucleotide is introduced to host cell with methods based on opening the lipid membrane of the target cells for example with electrical current and/or coupling the polynucleotides with a lipid envelope. The expression cassette may be in a plasmid encoding the CAR or as an mRNA encoding the CAR. The expression cassette may comprise parts enabling the integration to host cell. Any available non-viral gene delivery methods may be selected by skilled person. Such methods are for example transfection and nucleofection methods, use of liposomes, cationic agents and electroporation. Non-viral methods and their uses are reviewed by Harris et al 2020, Riedl et al 2018.

With viral methods a viral vector is used to introduce the CAR encoding polynucleotide of current invention into a host cell. Viral vector may be for example retroviral vector, lentiviral vector or adenoviral vector. The viral vector may be generated using plasmids containing the expression cassette comprising CAR encoding material, packaging material and envelope related material. Plasmids may be selected for example from pRRL.SIN-19, RSV-rev, pMDLg/pRRE and pMD.G. Other expression cassette materials may be selected from Chimeric 5′LTR-packaging signal—REV-responsive element—Promoter-Transgene cassette, REV expression plasmid, expression vector for precursor protein for matrix and capsid and nucleocapsid and precursor for reverse transcriptase and integrase components, expression vector for envelope protein e.g. VSV-G. Such plasmids would be introduced into the host cells resulting in the production of self inactiving viral particles containing the CAR expression cassette insert. Such a vector may integrate the cassette into the recipient cell genome. A skilled person may use any available viral based method to introduce polynucleotides encoding the CARs of current invention to a host cell. Viral vectors and related methods are described for example in references Dull et al 1998, Levine et al 2016.

### Cells

Host cell of the current invention means a cell expressing the CAR of the current invention. Polynucleotides encoding the CAR of current invention may be introduced into host cells via viral or non-viral methods. Host cell may be an eukaryotic cell or prokaryotic cell. Prokaryotic cell may be for example a bacterial cell. Eukaryotic cell may be for example animal cell, plant cell, fungal cell, insect cell. Host cell may be a cultured cell line. Such cell lines may be for example NK92 or Jurkat T cells. Host cell may be isolated from an organism for example animal, plant, fungus, insect. Preferably the host cell is isolated from human. The host cell may be for example blood cell, neuronal cell, epithelial cell, endothelial cell, hepatocyte. Preferably the host cell is blood cell, more preferably a leukocyte. The host cell may be a leukocyte selected from neutrophils, eosinophils, basophils, lymphocytes, monocytes. The host cell may be a lymphocyte selected from natural killer cell (NK), T lymphocyte (T cell) and/or B lymphocyte (B cell) or plasma cell. Preferably the host cell of current invention is T cell. T cell may be T helper (TH) cell, cytotoxic T (Tc) cell, Regulatory T (Treg) cell, natural killer T (NKT) cell. T cells may express specific cell surface molecules for example T cells CD3, TH cells CD4, Tc cells CD8. Different memory phenotypes are naïve T cell, T memory stem cell like (TSCM-like) cell, T central memory (TCM) cell, T memory stem cell (TSCM) cell, T effector (Teff) cell, T effector memory (TEM) cell. Memory phenotypes may be identified based on cell surface molecule expression e.g. CD95, CD45RO, CD45RA, CD27. Memory T cells and their surface markers are summarized in Table 2. Memory T cells may express CD4 or CD8. The host cell may comprise a single cell type or a population of different cell types, preferably the host cell is a specific T cell type or specific NK cell type, or a population comprising multiple T cell types and/or NK cell types. In current invention host cells may be a cell population of different cell types for example peripheral blood mononuclear cells isolated from blood sample. The host cells may be T cells isolated from peripheral blood mononuclear cells. T cells should be understood as cells expressing CD3 on their surface. The cells may also comprise natural killer T (NKT) cells, different T cell phenotypes, memory T cells, T helper cells, T effector cells, NK cells. The cells may specifically express for example cell surface markers like CD3, CD4, and/or CD8. Proportions of different cell types in cell populations may differ.

Cell populations may comprise T cells and NKT cells. Preferably the host cell population comprises more than 80%, 86% or 90% of T cells. Preferably the host cell population comprises less than 15%, 13% or 9% of NKT cells. In preferred embodiment the host cell population comprises more than 86% of T cells and less than 13% of NKT cells. T cells of the host cells may comprise for example CD4 positive and CD8 positive cells. The host cell population may comprise T cells, wherein less than 40% of the cells are CD57 positive and/or PD-1 positive.

A CAR of the invention, a polynucleotide encoding the spacer modified CAR, a vector comprising the polynucleotide encoding the spacer modified CAR and/or cells expressing a CAR of the current invention may be used to treat a disease associated to an antigen, which is targeted by the antigen binding domain of the CAR. The CAR binding to an antigen results to cytotoxicity of the antigen expressing target sell. Cells expressing CAR of the current invention may be used in a cell therapy of a cancer disease, preferably in a treatment of refractory and relapsed patients with hematological malignancies, acute lymphocytic leukemia (ALL), diffuse large B-cell lymphoma (DLBCL) and Non-Hodkin's lymphoma. Target antigens for the CAR expressing cells, preferably T cells, may be for example CD19, HER-2 and other cancer related target antigens selected for example from cancer associated antigens reviewed by Townsend et al 2018 and Yu et al 2020. Therapeutic CAR T cells may be used in cancer immunotherapy. Therapeutic CAR T cells may be autologous or allogeneic. Autologous cells are isolated from a patient, polynucleotide encoding the CAR is introduced to the cells by a vector and cells expressing the CAR is administered back to the patient. Allogeneic cells are isolated from a different individual but are genetically similar with cells of a patient.

CAR expressing cells, preferably T cells, may be administered to a patient in a pharmaceutical composition. The pharmaceutical composition may comprise in addition to CAR expressing cells, other pharmaceutically active agents, preservatives and/or buffer substances.

### EXAMPLES

### Example 1 Materials and Methods

### Design of the CARs

The sequence of the FMC63 antibody clone variable regions (Genbank: immunoglobulin light chain, variable region; CAA74660.1 and immunoglobulin heavy chain, variable region; CAA74659.1) were modified to design the CD19 targeting single chain variable fragment (scFv). The variable light chain and the variable heavy chain were joined with four canonical GGGGS-linkers. The hinge region from IgG1-CH1-domain was used to join the spacer to the CD19 binding domain. The spacer between the antigen binding domain and the cell membrane was constructed from SIRP-alpha Ig-like C1-type 1 and/or C1-type 2 domains. The SIRP-alpha primary structure was obtained from the Uniprot database (P78324) and reverse translated using Homo sapiens codons by means of estimated probabilities based on frequency distribution. Some spacer structure were constructed to include an additional extracellular fragment of T cell-specific surface glycoprotein CD28. The transmembrane (TM) and intracellular (IC) sequences were from the T cell-specific surface glycoprotein CD28 and from the intracellular T lymphocyte activation domain of the T cell receptor (TCR, CD3zeta-chain, Uniprot P20963-3, CD28, Uniprot P10747). Amino acid sequences of different CARs are summarized in Table 1.

Human Ab4D5 (Carter et al 1992) antibody clone was used to design the HER-2 targeting single chain variable fragment. In HER-2 targeting CAR construct other domains of the CAR were same as in CD19 targeting CAR M. HER-2 targeting CAR was prepared otherwise similarly as CD19 targeting CAR.

IgG1-based CAR (FMC63 scFv, IgG1-CH2-CH3 spacer, CD28 transmembrane and intracellular domains, and CD3zeta-signaling domain) was used as a positive control. FcR-binding site free control was CD28-based CAR (CAR XS; FMC63 scFv, IgG hinge region, extracellular, transmembrane and intracellular sequences from CD28 and intracellular sequences from CD3zeta-signaling domain). To evaluate the (CAR-) T cell specific interactions against target cells after transduction, a negative transduction control, an empty pLV-vector (mock) was used.

### T Cell Expansion

CAR T cells were manufactured from peripheral blood mononuclear cells separated from buffy coats as previously described (Kaartinen et al. 2017). In T cell cultures, X-VIVO (Lonza, Basel, Switzerland) media supplemented with 5% human AB-serum (Seralab, Oviedo, Spain) and 100 U/ml of IL-2 (Proleukin, Novartis, Basel, Switzerland) was used. T cell density was adjusted to 1×10⁶ cells/ml on days 0-2 and on day 3, after washing off the vector, the T cell density was adjusted to 0,5×10⁶ cells/ml by adding fresh culture medium. The T cells were transduced on day 2 using a third generation lentiviral vector (Koponen et al 2003) containing sequences encoding different CAR structures or mock vector. CAR T cells were cultured until day 10 and then frozen to await further analysis of cell functionality. For assessing the CAR T cell functionality, day 10 CAR T cells were thawed, adjusted to a cell density of 0,5×10⁶ cells/ml and cultured until day 13 before analysis. For memory phenotyping, CAR T cells were cultured until day 13 without freezing.

### Cell Lines

NALM-6 (CD19+ B lineage, acute lymphoblastic leukemia, ALL) cells, THP-1 (FcR+monocytes, acute monocytic leukemia) cells and E6.1 Jurkat T cells were cultured in RPMI-1640 medium (Thermo Fisher Scientific, Waltham, USA) supplemented with 10% fetal bovine serum (Thermo Fisher Scientific), 100 IU/mL penicillin and 100 μg/mL streptomycin (Thermo Fisher Scientific). In addition for Jurkat T cells, 2 mM L-glutamine was added. The NALM-6-luc cell line was generated as described in Dufva et al 2019.

### Flow Cytometry

The cells were fixed with 1% paraformaldehyde (10 min, +4° C.) prior to staining with anti-human antibodies. As a control fluorescence minus one (FMO) and/or appropriate isotype controls were used. Samples were run on a BD FACSAria Ilu cytometer (BD Biosciences, Franklin Lakes, USA) and the results analyzed using FlowJo (version 10.5.3, BD Biosciences) software.

### Memory Phenotyping of CAR T Cells

After expansion, T cell subtypes and residual NK- and NKT cells (Table 2) were stained using following anti-human antibodies from BD Biosciences: CD3 (clone UCHT1)-Fluorescein isothiocyanate (FITC), CD4 (clone SK3)-BD Horizon™ Brilliant Violet™ 510 (BV510), CD8 (RPA-T8)-BD Horizon™ Brilliant Violet™ 421 (BV421), CD56 (clone B159)-Allophycocyanin (APC). Memory T cell phenotypes were identified using CD27 (clone M-T271)-Peridinin-chlorophyll protein (PerCP) conjugated with Cyanine 5.5 (Cy 5.5), CD45RA (clone HI100)-APC, CD45RO (clone UCHL1)-Phycoerythrin (PE) conjugated with Cyanine 7 (Cy7) and CD95 (clone DX2)-PE.

The T cell memory phenotypes were defined using expression markers shown in Table 2 for CD4 and CD8 subpopulations. To specify the T cell maturation into a terminal effector-phenotype and exhaustion, antibodies for CD57 (clone NK-1)-BD Horizon™ Brilliant Violet™ 421 (BV421) and CD279 (clone MIH4)-AF647 were used. The expression of programmed cell death protein 1 (CD279) and T cell terminal effector inducing marker CD57 was assessed in the CD95+ CD27+/−CD45RO+/− populations. CAR-expression was measured using a F(ab′)2 fragment goat-antihuman immunoglobulin (Ig)G(H+L) conjugated with Alexa Fluor® 647 (Jackson Immunoresearch, Inc West Grove, USA.).

### Cytotoxicity Assay

To assess the cytotoxic efficacy of spacer modified CARs, the cells were co-cultured with Luc+NALM-6 cells at various T cell:B cell ratios (effector:target-ratios, E:T) for 18 hours. At the end of the co-culture, luciferin (ONE-Glo Luciferase reagent, Promega) was added and the presence of live target cells was quantified according to the manufacturer's instructions with a CLARIOstar Plus Multi-Mode Microplate Reader (BMG Labtech).

### Degranulation Assay

To measure target cell-induced degranulation of T cells, cells were co-cultured with NALM-6 target cells at 1:1 (E:T) ratio for 4 hours in the presence of lysosomal- associated membrane protein 1 (CD107a) antibody (PE conjugated, clone H4A3, BD Biosciences) and GolgiStop™ Protein Transport Inhibitor (BD Biosciences). Degranulation was assessed as a proportion of cell surface expressing CD107a⁺ T cells from total T cells in co-cultures measured with flow cytometry.

Analyses Demonstrating CAR T Cell Interactions with Monocytes

To analyze the effects of CAR T cell binding to monocytes, T cells were co-cultured with THP-1 monocytes at 1:1 ratio for 18 h at +37° C. The cell surface activation markers CD25 (clone BC96, BioLegend) and CD69 (clone FN50, BD Biosciences) on T cells were measured using flow cytometry and the cell culture media were collected for further analyses of activation induced cytokines (monocytes: IL-1beta and CAR T cells: IFN-gamma and IL-2).

### Cytokine Assay

To quantify activation induced cytokines from cytotoxicity assays (IFN-gamma and IL-2) and from analyses demonstrating CAR T cell interactions with monocytes (IFN-gamma, IL-2 and IL-1B), cell culture media (effector:target ratio; 1:1) were analyzed Cytometric Bead Array (CBA Human Soluble Protein Master Buffer Kit together with IL-2, IFN-gamma and IL-1beta CBA Flex Sets, BD Biosciences) according to the manufacturers' instructions. Results were analyzed using FCAP Array Software v 3.0 (BD Biosciences).

### Antibody Conjugation and Magnetic Microbead Selection of CAR Positive Jurkat T Cells

SIRP-alpha binding antibody (SE12136; Seiffert et al. 2001) was conjugated with Cyanine 5 (Cy5) fluorochrome using LYNX Rapid Plus Cy5 Antibody Conjugation Kit (Bio-Rad, Hercules, USA) according to manufacturer's instructions. Jurkat T cells were selected utilizing single cell separation (Anti-Cy5/Anti-Alexa Fluor 647 MicroBeads, Miltenyi Biotec) according to manufacturer's instructions and the expression was confirmed by flow cytometry.

### Example 2 T Cell Expansion and CAR Expression

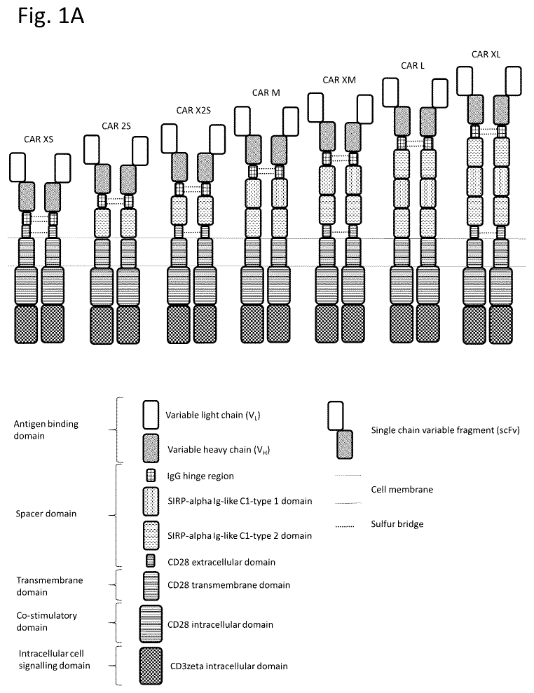

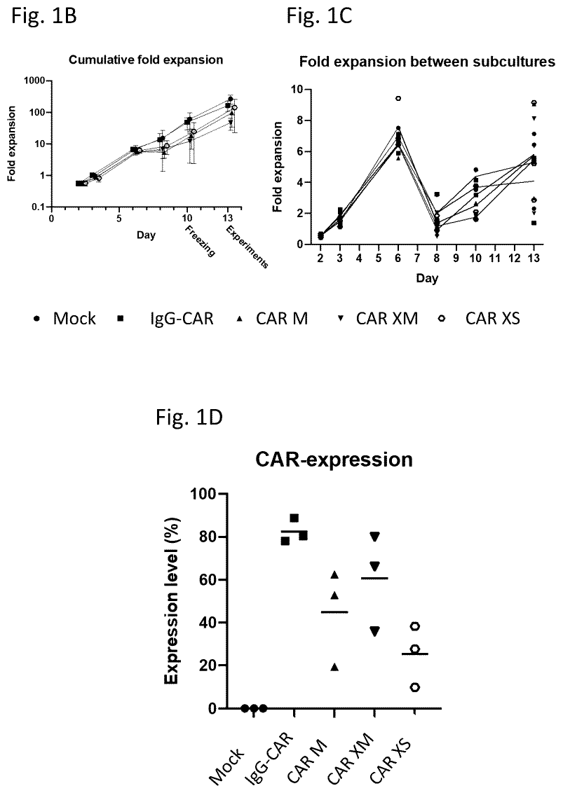

CAR constructs CAR XS, CAR XM, and CAR M comprising an scFv part from the monoclonal antibody FMC63, the extracellular spacer from Ig-like C1-type 1 and Ig-like C1-type 2 domains of SIRP-alpha, IgG hinge region and/or CD28, transmembrane domain from CD28 and intracellular domain from CD28 and CD3zeta (FIG. 1A). CARs were transduced into T cells using a lentiviral vector (pLV) under hPGK-promoter (Koponen JK et al 2003).

Different CAR-transduced T cells expanded 48-260 fold within 13 days (FIG. 1B). There were no significant differences in expansion rates, however CAR XM transduced cells showed a tendency for slower growth. Even though the differences in growth data can be seen in early phase, CARs M and XM appears to have a characteristic second peak in growth (FIG. 1C) after thawing the cells at day 10 in contrast to IgG1-CAR.

On day two of expansion, the T cells were stably transduced with lentiviruses carrying CAR genes or with mock vectors. After manufacturing the cells, on day 13 we analyzed the cells for CAR expression which was detected in 25.3% to 88.8% of the cells (mean±SD; IgG1-CAR 88.8±5.6, CAR M 45.0±22.6, CAR XM 60.6±22.6 and CAR XS 25.3±14.3) as measured by subtracting the CAR antibody binding results of empty vector-transduced T cells (Mock 13.25±5.2) (FIG. 1D).

All CARs were successfully expressed on T cells, but after day 6 of culture the expansion rate of the CAR XS, CAR M and CAR XM T cells appeared to be somewhat lower than that of IgG-CAR and mock T cells (FIG. 1C).

### Example 3 Characterization of the T Cellphenotypes and Maturation Between the Different CAR Expressing T Cells

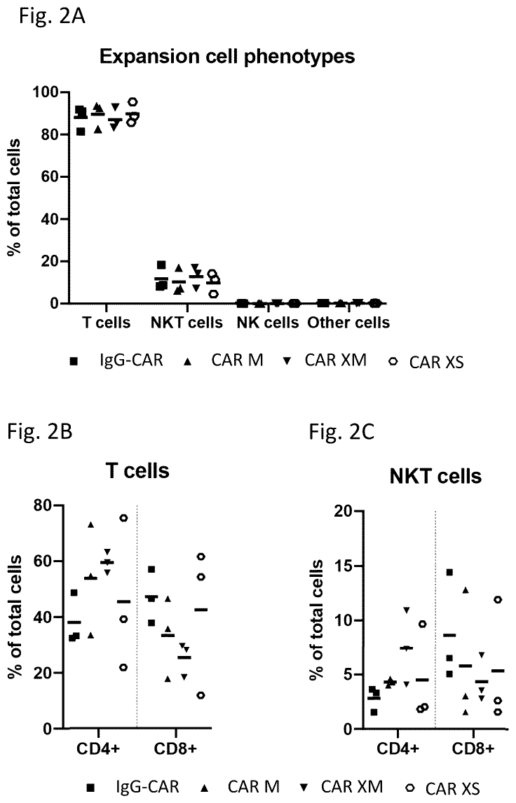

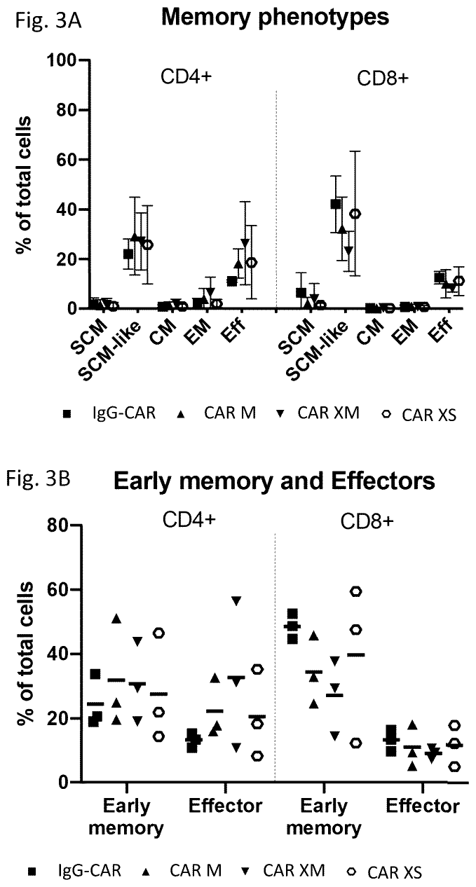

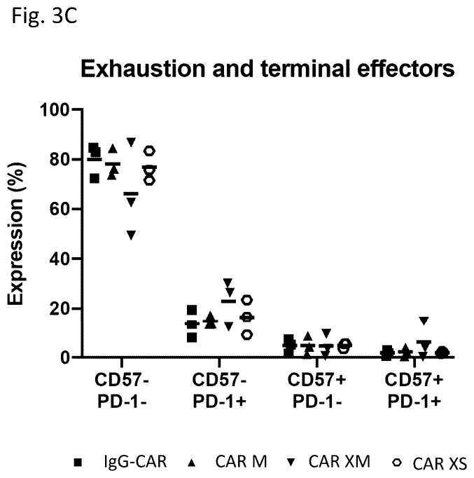

After 13 days of expansion, the majority of the cells (86%-90%) were T cells (CD3+ CD56−) including 9-13% NKT cells (CD3+ CD56+), with very little additional contribution by NK cells (CD3− CD56+) or residual CD3− CD56-cells (FIG. 2A). In addition to cell phenotypes, we then evaluated the T cell memory phenotypes. Earlier we reported that the concentration of IL-2 during CAR T-cell expansion influences T cell memory phenotype (Kaartinen T et al 2017). Accordingly, we used 100 U/ml IL-2 in the cultures to prevent excessive differentiation of the T cells. The repertoire of T cell memory phenotypes are shown in FIG. 3A. To more easily distinguish the effects of the expansion process and the various CARs on the memory phenotype of the T cells, we grouped the T cell memory subgroups into Early memory (=Tscm, Tscm-like and Tcm) and Effector (=Tem and Teff) groups (FIG. 3B). By day 13 of expansion T cells equipped with the SIRP-alpha based CAR M and CAR XM tended to favor differentiation toward CD4+ cells (FIG. 2B), of which the proportion of Effector T cells tended to be higher in contrast to the CD8 cells showing a stronger preponderance of early memory cells. Nevertheless, because of the variations between different donors, no statistically significant differences in T memory cell differentiation related to the various CARs were detected. Otherwise, memory phenotypes remained the same as well as the exhaustion levels measured with PD-1 surface expression and proliferation capacity with T cell terminal effector maturation associated marker CD57. After 13-day expansion, most of the CD4 and CD8 cells were negative for the exhaustion markers CD57 and PD-1 (66.2-79.9%), with a minority expressing one or both of the surface markers (FIG. 3C). Again, the CARs did not differentially influence exhaustion marker expression in T cells.

### Example 4 Activation and Cytotoxic Activity of the CAR T Cells

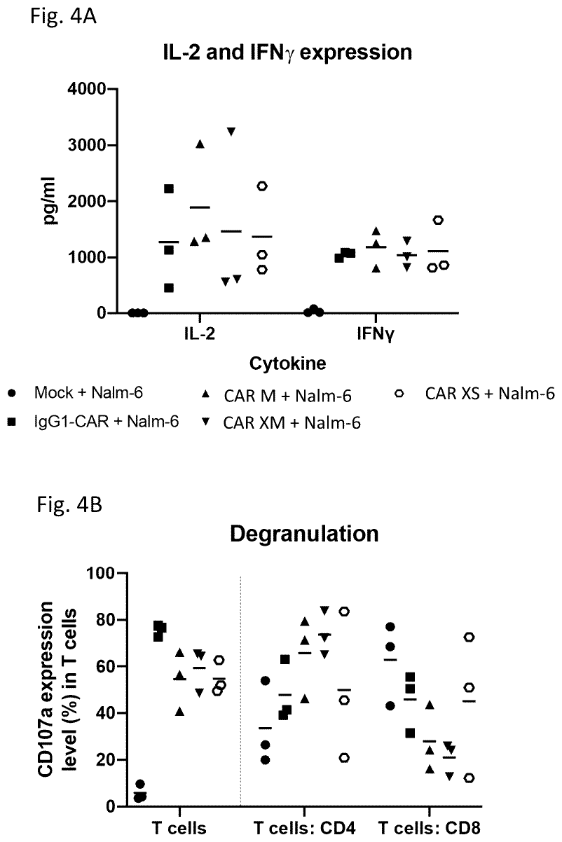

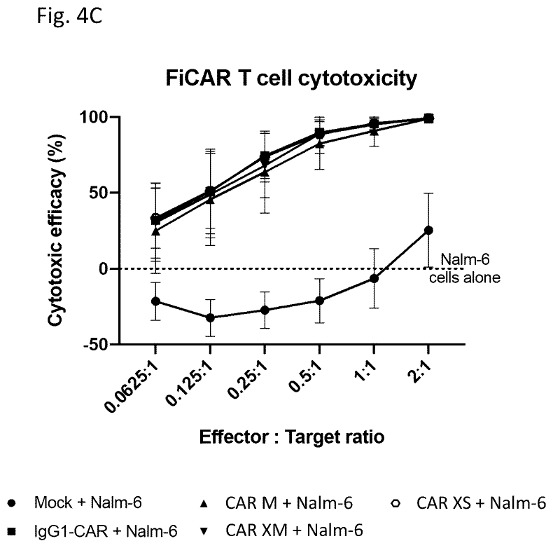

CAR T cell interaction with cells carrying the target antigen induces T cell activation and target cell killing. Having established that T cells carrying the spacer modified CAR constructs can successfully be generated, we next analyzed the functional characteristics of the CAR T cells in response to target-dependent activation. To analyze CAR function in T cell activation in response to CD19+ target cells, we measured cytokine production from overnight co-cultures using 1:1 effector:target cell-ratios (FIG. 4A). All the T cells carrying the different CARs, produced similar amounts of IL-2, with a non-significant tendency of higher IL-2 production (−1.3 fold more) by CAR M carrying cells. No differences in IFN-gamma production were detected.

We then investigated the ability of the T cells to degranulate in response to a 4-hour co-culture with CD19+ target cells by measuring the appearance of cell surface expression of CD107a. The proportion of CAR expressing cells was directly linked to the fraction of degranulating cells in response to target cells (FIG. 4B), confirming the functionality of CAR expressing cells. Although, the CAR expression levels with IgG1-CAR were higher than in CAR M, CAR XM or CAR XS, the CD107a expression of CD4+ cells were alike. In contrast, the IgG1-CAR and CAR XS showed higher expression of CD107a in CD8+ cells than CAR M and CAR XM.

Despite differing CAR expression and CD8+ cell degranulation levels, all CAR T cells displayed remarkably similar cytotoxic efficacy against NALM-6 cell targets (FIG. 4C). In an 18 h co-culture experiment with CD19+ target cells, all of the CAR T cell lines demonstrated 100% killing efficacy at a 2:1 (E:T) ratio and similar proficiencies also at lower E:T ratios.

### Example 5 Spacer Modified CAR T Cells Showed No Activation with “Off-target” Myeloid Cells

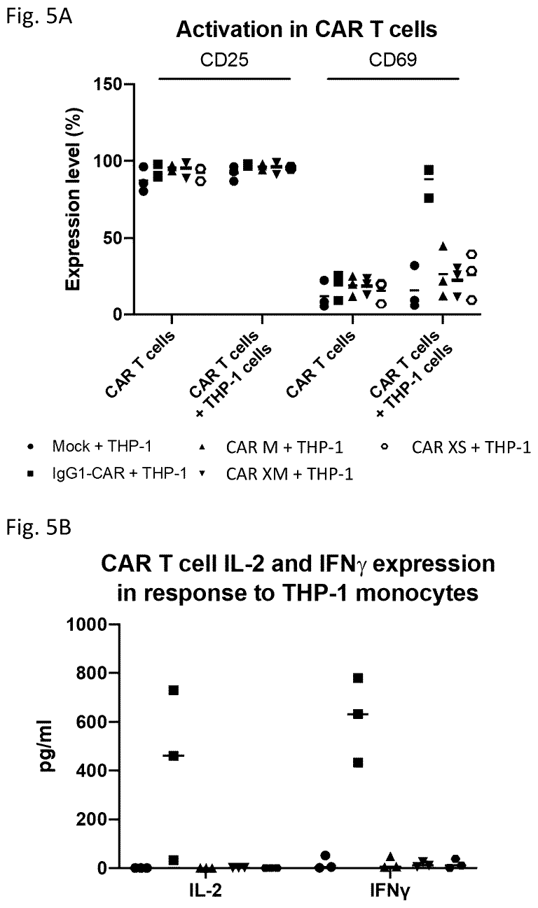

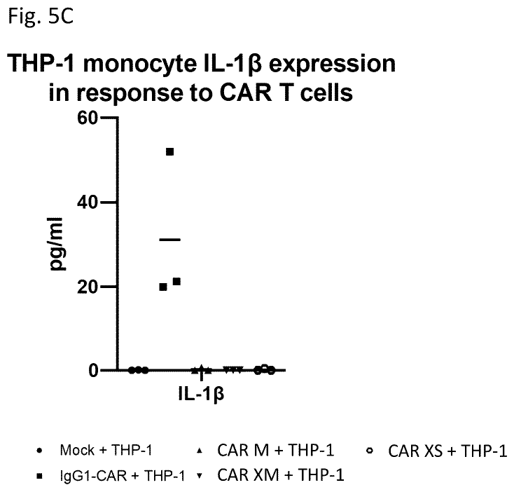

SIRP-alpha based FiCARs were designed to escape interactions with Fc-receptor expressing myeloid cells. We evaluated the CAR T cell interactions with myeloid cells by co-culturing CART cells with THP-1 monocytes at a 1:1 (effector:off-target cell; E:OT) ratio. CART cell activation was measured by staining for cell surface activation markers CD25 that indicated long-term activation and CD69 for short-term activation (FIG. 5A) and by measuring cytokines produced by T cells (FIG. 5B: CAR T cells: IFN-gamma and IL-2) and monocytes in response to CAR-related activation (FIG. 5C: monocytes: IL-1beta). All of the CART cells expressed high and equivalent levels of the CD25 activation marker with or without THP-1 monocytes. Furthermore, in co-cultures with CAR T cells and THP-1 monocytes, the Fc-region-containing CAR, the IgG1-CAR, expressed high levels of cell surface early activation marker CD69. In contrast, T cells with spacer modified CAR constructs, namely CAR XS, CAR M and CAR XM, cells did not show CD69 expression in conjunction with Mock T cells. Similar setting can be seen in cytokine production, in which the IgG-CAR produced activation induced cytokines IL-2 and IFN-gamma in addition to THP-1 produced activation induced cytokine IL-1beta. Again, spacer modified CART cells produced low levels of IL-2 and IFN-gamma that are all equal to mock-transduced control T cells with or without THP-1 monocytes. Furthermore, THP-1 monocytes in co-culture with spacer modified T cells, the THP-1 monocytes produced low levels of IL-1beta that is equal to THP-1 cells alone or with mock-transduced control T cells. Taken together, these data indicate that co-culture of spacer modified CAR T cells with FcR-carrying monocytes does not lead to undue activation of the T cells nor of the monocytes.

### Example 6 Modifying the SIRP-alpha Spacer Length

To further investigate whether the CAR backbone structure may be modified for a better binding of membrane proximal or membrane distal antigens on target cells, we designed various length CARs to target CD19. By adjusting the spacer length utilizing the different Ig-like C1 domains of SIRP-alpha, we designed length-adjusted CARs by removing another of the Ig-like C1 domains from CAR M or CAR XM or by adding an extra Ig-like C1 domain to the CAR M and CAR XM.

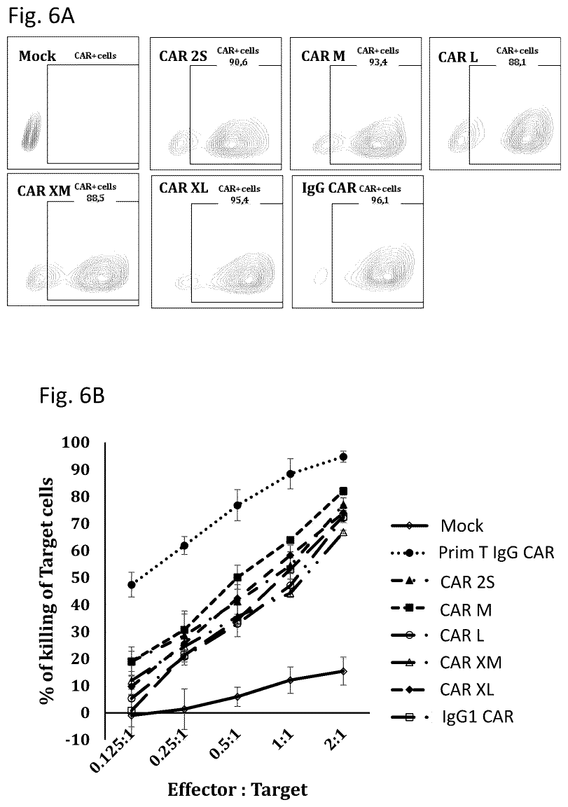

First, to assure the high expression of different length CARs, CAR expressing Jurkat T cells were selected using single cell microbead separation. Then, to measure the expression, the various length CARs were stained using biotinylated antihuman CD19 CAR Detection Reagent (Miltenyi Biotec) and a Biotin antibody conjugated with APC (Miltenyi Biotec) as a secondary antibody. The staining was performed according to manufacturer's instructions. All the transduced Jurkat T cell cultures displayed high expression levels of different CARs (FIG. 6A: CAR 2S 90,6%, CAR L 88,1%, CAR XL 95,4%) in contrast to empty vector transduced mock Jurkat T cells showing no unspecific binding of antibodies.

Furthermore, to assess the functionality of various length CARs, we tested the cytotoxic efficacy of CAR-transduced Jurkat T cells against CD19 positive Nalm-6-luc cells in several E:T ratios (FIG. 6B). Jurkat T cells expressing each of the various CARs (CAR 2S, CAR L and CAR XL) all displayed similar killing efficacy in as did the Jurkat T cells equipped with CAR M, CAR XM and IgG1-based control CAR ; from 0-20% to 66.8-82% depending on the E:T ratio. As a positive control for the killing, we used primary T cells that showed superior killing efficacy (48-94,7%) and as negative control mock transduced Jurkat T cells showing non-CAR related killing efficacy of 0-15.4% at different E:T -ratios.

### Example 7 Targeting HER-2 with CAR Based on SIRPalpha Backbone

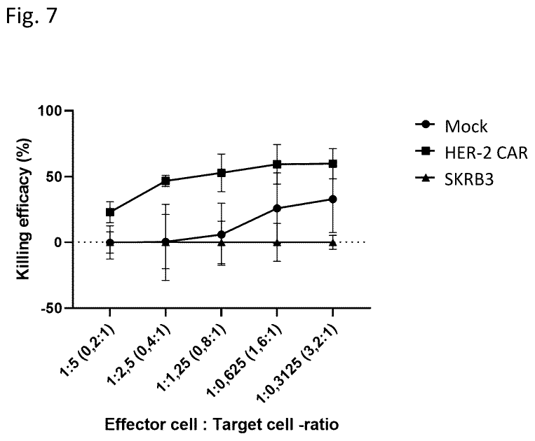

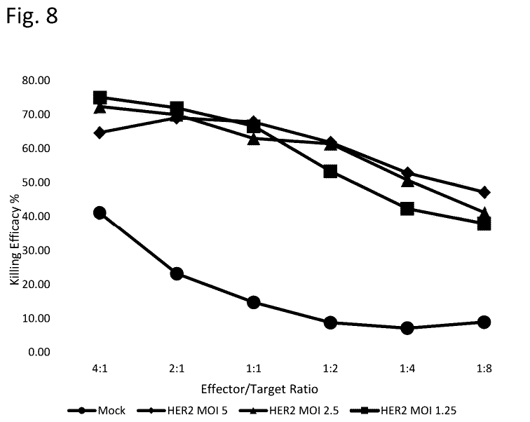

After demonstrating that the spacer length can be adjusted, we designed a new CAR targeting HER-2 by replacing the CD19-targeting scFv domain in the previous CAR M structure with a HER-2 targeting ScFv domain. To demonstrate the function of HER-2 targeting CAR M, the CAR was transduced into primary T cells. After expansion, the HER2 targeting CAR T cells were co-cultured with HER-2 positive SKBR-3-eGFP-luc breast carcinoma cells at various effector-target (E:T) ratios (FIG. 7). In 18 h cytotoxic preliminary testing (n=1), T cells transduced with HER-2 targeting CAR M showed higher killing efficacy compared to mock-transduced T cells, that by themselves displayed minor CAR-independent cell killing.

### Example 8 Cytotoxicity of T Cells Expressing CAR M with a scFv Targeting HER-2

T cells were isolated from healthy donor buffy coats, transduced with lentiviral vectors carrying the HER-2 CAR M gene construct using different multiplicities of infection (MOI) 1,25, 2,5 and 5, and expanded for 11 days. T cells expressing HER-2 CAR M with an alternative scFv targeting HER-2 (effector cells) were incubated together with firefly luciferase-expressing HER-2+ SKBR3 breast carcinoma cells (target cells) at the effector-target (E:T) ratios 4:1, 2:1, 1:1, 1:2, 1:4 and 1:8. After 24 hours luciferin was added and the live target cells were quantified showing high killing efficacy with all the different E:T ratios compared to empty vector (mock) transduced T cells.

### Example 9 Cell Expansion, CAR Expression and Cytotoxicity of CAR Constructs with Modified Multimerization Domains

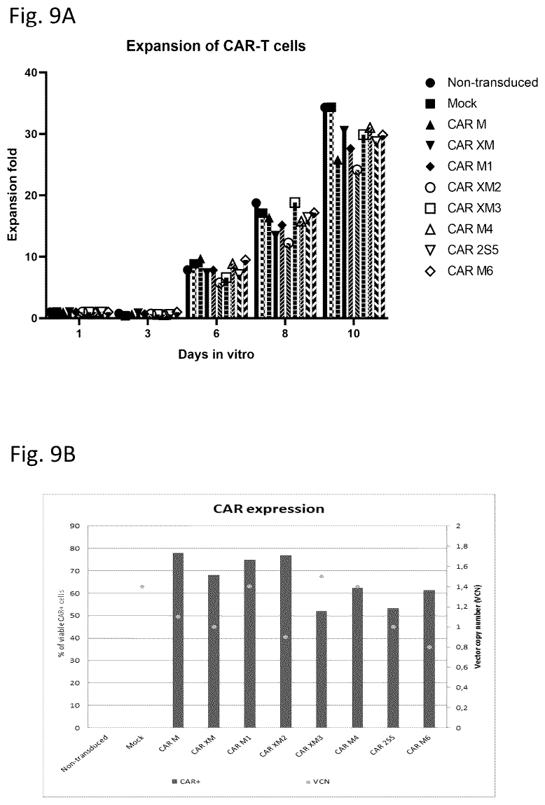

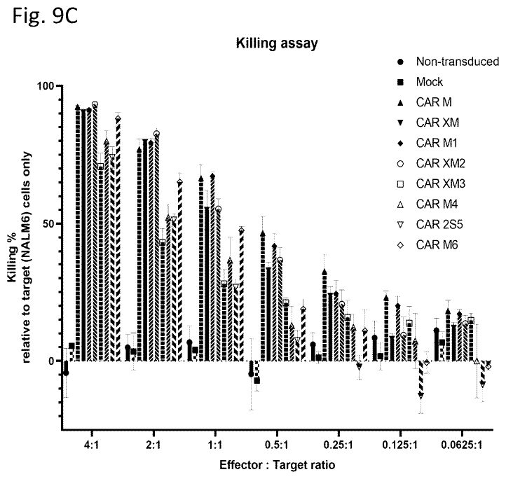

CD4+ and CD8+ T cells were purified from peripheral blood mononuclear cells with magnetic beads (Miltenyi Biotec). Purified CD4+ and CD8+ T cells were transduced with lentivirus vectors encoding CAR constructs (CAR M, CAR, XM, CAR Ml, CAR XM2, CAR XM3, CAR M4, CAR 2S5, CAR M6) and expanded in culture medium containing IL-7 and IL-15 (Miltenyi Biotec) at 12,5 ng/ml. Cell amounts and viability were measured during the expansion. Different CAR constructs were studied for the effect on expansion (FIG. 9A) up until day 10. The different constructs did not clearly have an effect on the cell expansion and all the constructs reached over 20-fold expansion.

The cells were also studied for their CAR expression with flow cytometry. The CAR constructs were detected by a biotin labelled antibody detecting a specific domain present in all the CAR constructs (FIG. 9B). The vector copy number (VCN) was studied by isolating the genomic DNA and detecting the integrated gene with transgene specific primers (FIG. 9B). With a VCN of roughly 1 in the cell population, over 50% of cells expressed CAR transgene on the surface of the cell.

The CAR-T cells (post thaw) were co-cultured with CD19+ NALM-6 target cells with different ratios of effector (CAR-T) and target (cancer) cells for 24 hours. At this point the cells were lysed and measured for target cell specific (trans)gene activity (FIG. 9C). In the killing assay CAR M, XM, M1 and M6 showed tendency of higher killing efficacy than other CAR constructs, but all constructs displayed significantly elevated killing efficacy of target cells compared to non-transduced or empty vector (mock) transduced T cells.

### REFERENCES

Almåsbak H et al (2015) Inclusion of an IgG1-Fc spacer abrogates efficacy of CD19CART cells in a xenograft mouse model. Gene Ther 22: 391-403.

Casucci M et al (2018) Extracellular NGFR spacers allow efficient tracking and enrichment of fully functional car-t cells co-expressing a suicide gene. Front Immunology 9

Carter P et al (1992) Humanization of an anti-p185HER2 antibody for human cancer therapy. Proc Natl Acad Sci U S A 89:4285-4289.

Dull et al (1998) A Third-Generation Lentivirus Vector with a Conditional Packaging System. J Virol 72 (11): 8463-8471.

Harris E et al (2020) Optimization of electroporation and other non-viral gene delivery strategies for T cells. Biotechnol Progress, e3066.

Hatherley D et al (2007) The structure of the macrophage signal regulatory protein α (SIRP-alpha) inhibitory receptor reveals a binding face reminiscent of that used by T cell receptors. J Biol Chem 282: 14567-14575.

Hatherley D et al (2009) Structure of signal-regulatory protein a: A link to antigen receptor evolution. J Biol Chem 284: 26613-26619.

Hombach A et al (2010) Adoptive immunotherapy with genetically engineered T cells: modification of the IgG1 Fc ‘spacer’ domain in the extracellular moiety of chimeric antigen receptors avoids ‘off-target’ activation and unintended initiation of an innate immune response. Gene Ther 17: 1206

Hudecek M et al (2015) The Nonsignaling Extracellular Spacer Domain of Chimeric Antigen Receptors Is Decisive for In Vivo Antitumor Activity. Cancer Immunol Res 3: 125-135.

Kaartinen T, Luostarinen A, Maliniemi P, et al (2017) Low interleukin-2 concentration favors generation of early memory T cells over effector phenotypes during chimeric antigen receptor T-cell expansion. Cytotherapy 19:689-702.

Koponen J K et al (2003) Doxycycline-regulated lentiviral vector system with a novel reverse transactivator rtTA2S-M2 shows a tight control of gene expression in vitro and in vivo. Gene Ther 10: 459-466.

Levine B et al (2017) Global Manufacturing of CAR T Cell Therapy. Molecular Therapy: Methods a Clinical Development Vol. 4: 92-101

Riedl S et al (2018) Non-Viral Transfection of Human T Lymphocytes. Processes, 6, 188.

Seiffert M et al (2001) Signal-regulatory protein a (SIRPα) but not SIRPβ is involved in T-cell activation, binds to CD47 with high affinity, and is expressed on immature CD34+CD38− hematopoietic cells. Blood 97:2741-2749.

Sentman C L et al (2014) NKG2D CARs as Cell Therapy for Cancer. Cancer J 20: 156-159.

Townsend M H et al (2018) The expansion of targetable biomarkers for CAR T cell therapy. Journal of Experimental a Clinical Cancer Research 37:163

van Beek E M et al (2005) Signal Regulatory Proteins in the Immune System. J. Immunol. 175: 7781-7.

Yu J X et al (2020) Cancer cell therapies: the clinical trial landscape. Nature Reviews Drug Discovery 19, 583-584.

TABLE 1
Summary of amino acid sequences and nucleic acid sequences
Abbreviations (T) sequence type; (a) protein or peptide sequence
comprising amino acids; (n) polynucleotide sequence comprising
nucleic acids; (SP) species; (h) homo sapiens (a) artificial

| Description | Sequence | T | SP | ID no |
|---|---|---|---|---|
| SIRP-alpha Ig- | PSAPVVSGPAARATPQHTVSFTCESHGFSPRDITLKWFKNGNELSDFQ | a | h | 1. |
| like C1 type 1 | TNVDPVGESVSYSIHSTAKVVLTREDVHSQVICEVAHVTLQGDPLRGTA | | | |
| | NLS | | | |
| SIRP-alpha Ig- | PTLEVTQQPVRAENQVNVTCQVRKFYPQRLQLTWLENGNVSRTETAS | a | h | 2. |
| like C1 type 2 | TVTENKDGTYNWMSWLLVNVSAHRDDVKLTCQVEHDGQPAVSKSHD | | | |
| | LK | | | |
| fragment of | KGKHLCPSPLFPGPSKP | a | h | 3. |
| extracellular | | | | |
| CD28 domain | | | | |
| IgG1 hinge | YVTVSSQDPAEPKSPDKTHTCPPCP | a | h | 4. |
| region | | | | |
| linker | GGGGS | a | a | 5. |
| linker | GGGGSVP | a | a | 6. |
| linker | GGGGSAK | a | a | 7. |
| linker | VSGGGGS | a | a | 8. |
| linker C1 | ETIRVP | a | a | 9. |
| CAR spacer XS | YVTVSSQDPAEPKSPDKTHTCPPCPKGKHLCPSPLFPGPSKP | a | a | 10. |
| CAR spacer 1S | YVTVSSQDPAEPKSPDKTHTCPPCPGGGGSAKPSAPVVSGPAARATPQ | a | a | 11. |
| | HTVSFTCESHGFSPRDITLKWFKNGNELSDFQTNVDPVGESVSYSIHST | | | |
| | AKVVLTREDVHSQVICEVAHVTLQGDPLRGTANLS | | | |
| CAR spacer 2S | YVTVSSQDPAEPKSPDKTHTCPPCPGGGGSVPPTLEVTQQPVRAENQ | a | a | 12. |
| | VNVTCQVRKFYPQRLQLTWLENGNVSRTETASTVTENKDGTYNWMS | | | |
| | WLLVNVSAHRDDVKLTCQVEHDGQPAVSKSHDLKVS | | | |
| CAR spacer | YVTVSSQDPAEPKSPDKTHTCPPCPGGGGSAKPSAPVVSGPAARATPQ | a | a | 13. |
| X1S | HTVSFTCESHGFSPRDITLKWFKNGNELSDFQTNVDPVGESVSYSIHST | | | |
| | AKVVLTREDVHSQVICEVAHVTLQGDPLRGTANLSGGGGSKGKHLCPS | | | |
| | PLFPGPSKP | | | |
| CAR spacer | YVTVSSQDPAEPKSPDKTHTCPPCPGGGGSVPPTLEVTQQPVRAENQ | a | a | 14. |
| X2S | VNVTCQVRKFYPQRLQLTWLENGNVSRTETASTVTENKDGTYNWMS | | | |
| | WLLVNVSAHRDDVKLTCQVEHDGQPAVSKSHDLKVSKGKHLCPSPLFP | | | |
| | GPSKP | | | |
| CAR spacer M | YVTVSSQDPAEPKSPDKTHTCPPCPGGGGSAKPSAPVVSGPAARATPQ | a | a | 15. |
| | HTVSFTCESHGFSPRDITLKWFKNGNELSDFQTNVDPVGESVSYSIHST | | | |
| | AKVVLTREDVHSQVICEVAHVTLQGDPLRGTANLSETIRVPPTLEVTQQ | | | |
| | PVRAENQVNVTCQVRKFYPQRLQLTWLENGNVSRTETASTVTENKDG | | | |
| | TYNWMSWLLVNVSAHRDDVKLTCQVEHDGQPAVSKSHDLKVS | | | |
| CAR spacer XM | YVTVSSQDPAEPKSPDKTHTCPPCPGGGGSAKPSAPVVSGPAARATPQ | a | a | 16. |
| | HTVSFTCESHGFSPRDITLKWFKNGNELSDFQTNVDPVGESVSYSIHST | | | |
| | AKVVLTREDVHSQVICEVAHVTLQGDPLRGTANLSETIRVPPTLEVTQQ | | | |
| | PVRAENQVNVTCQVRKFYPQRLQLTWLENGNVSRTETASTVTENKDG | | | |
| | TYNWMSWLLVNVSAHRDDVKLTCQVEHDGQPAVSKSHDLKVSKGKH | | | |
| | LCPSPLFPGPSKP | | | |
| CAR spacer L | YVTVSSQDPAEPKSPDKTHTCPPCPGGGGSVPPTLEVTQQPVRAENQ | a | a | 17. |
| | VNVTCQVRKFYPQRLQLTWLENGNVSRTETASTVTENKDGTYNWMS | | | |
| | WLLVNVSAHRDDVKLTCQVEHDGQPAVSKSHDLKVSGGGGSAKPSAP | | | |
| | VVSGPAARATPQHTVSFTCESHGFSPRDITLKWFKNGNELSDFQTNVD | | | |
| | PVGESVSYSIHSTAKVVLTREDVHSQVICEVAHVTLQGDPLRGTANLSE | | | |
| | TIRVPPTLEVTQQPVRAENQVNVTCQVRKFYPQRLQLTWLENGNVSR | | | |
| | TETASTVTENKDGTYNWMSWLLVNVSAHRDDVKLTCQVEHDGQPAV | | | |
| | SKSHDLKVS | | | |
| CAR spacer XL | YVTVSSQDPAEPKSPDKTHTCPPCPGGGGSVPPTLEVTQQPVRAENQ | a | a | 18. |
| | VNVTCQVRKFYPQRLQLTWLENGNVSRTETASTVTENKDGTYNWMS | | | |
| | WLLVNVSAHRDDVKLTCQVEHDGQPAVSKSHDLKVSGGGGSAKPSAP | | | |
| | VVSGPAARATPQHTVSFTCESHGFSPRDITLKWFKNGNELSDFQTNVD | | | |
| | PVGESVSYSIHSTAKVVLTREDVHSQVICEVAHVTLQGDPLRGTANLSE | | | |
| | TIRVPPTLEVTQQPVRAENQVNVTCQVRKFYPQRLQLTWLENGNVSR | | | |
| | TETASTVTENKDGTYNWMSWLLVNVSAHRDDVKLTCQVEHDGQPAV | | | |
| | SKSHDLKVSKGKHLCPSPLFPGPSKP | | | |
| Variable light | DIQMTQTTSSLSASLGDRVTISCRASQDISKYLNWYQQKPDGTVKLLIY | a | h | 19. |
| chain of anti- | HTSRLHSGVPSRFSGSGSGTDYSLTISNLEQEDIATYFCQQGNTLPYTF | | | |
| CD19 scFv | GGGTKLELKR | | | |
| Variable heavy | EVQLQQSGPGLVAPSQSLSVTCTVSGVSLPDYGVSWIRQPPRKGLEWL | a | h | 20. |
| chain of anti- | GVIWGSETTYYNSALKSRLTIIKDNSKSQVFLKMNSLQTDDTAIYYCAKH | | | |
| CD19 scFv | YYYGGSYAMDYWGQGTTVTVSS | | | |
| Linker | GGGGSGGGGSGGGGSGGGGS | a | a | 21. |
| scFV anti-CD19 | DIQMTQTTSSLSASLGDRVTISCRASQDISKYLNWYQQKPDGTVKLLIY | a | a | 22. |
| | HTSRLHSGVPSRFSGSGSGTDYSLTISNLEQEDIATYFCQQGNTLPYTF | | | |
| | GGGTKLELKRGGGGSGGGGSGGGGSGGGGSEVQLQQSGPGLVAPS | | | |
| | QSLSVTCTVSGVSLPDYGVSWIRQPPRKGLEWLGVIWGSETTYYNSAL | | | |
| | KSRLTIIKDNSKSQVFLKMNSLQTDDTAIYYCAKHYYYGGSYAMDYWG | | | |
| | QGTTVTVSS | | | |
| CD28 trans- | FWVLVVVGGVLACYSLLVTVAFIIFWV | a | h | 23. |
| membrane | | | | |
| domain | | | | |
| CD28 | RSKRSRLLHSDYMNMTPRRPGPTRKHYQPYAPPRDFAAYRS | a | h | 24. |
| intracellular | | | | |
| domain | | | | |
| CD3zeta | RVKFSRSADAPAYQQGQNQLYNELNLGRREEYDVLDKRRGRDPEMGG | a | h | 25. |
| | KPRRKNPQEGLYNELQKDKMAEAYSEIGMKGERRRGKGHDGLYQGLS | | | |
| | TATKDTYDALHMQALPPR | | | |
| CAR XS | MEFGLSWLFLVAILKGVQCSRDIQMTQTTSSLSASLGDRVTISCRASQDI | a | a | 26. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPKGKHLCPSPLFPGPSKPFWVLVVVGGVLACYSLLVTVAFIIFWVR | | | |
| | SKRSRLLHSDYMNMTPRRPGPTRKHYQPYAPPRDFAAYRSRVKFSRSA | | | |
| | DAPAYQQGQNQLYNELNLGRREEYDVLDKRRGRDPEMGGKPRRKNP | | | |
| | QEGLYNELQKDKMAEAYSEIGMKGERRRGKGHDGLYQGLSTATKDTY | | | |
| | DALHMQALPPR | | | |
| CAR 1S | MEFGLSWLFLVAILKGVQCSRDIQMTQTTSSLSASLGDRVTISCRASQDI | a | a | 27. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSAKPSAPVVSGPAARATPQHTVSFTCESHGFSPRDITLKW | | | |
| | FKNGNELSDFQTNVDPVGESVSYSIHSTAKVVLTREDVHSQVICEVAHV | | | |
| | TLQGDPLRGTANLSGGGGSFWVLVVVGGVLACYSLLVTVAFIIFWVRS | | | |
| | KRSRLLHSDYMNMTPRRPGPTRKHYQPYAPPRDFAAYRSRVKFSRSAD | | | |
| | APAYQQGQNQLYNELNLGRREEYDVLDKRRGRDPEMGGKPRRKNPQ | | | |
| | EGLYNELQKDKMAEAYSEIGMKGERRRGKGHDGLYQGLSTATKDTYD | | | |
| | ALHMQALPPR | | | |
| CAR 2S | MEFGLSWLFLVAILKGVQCSRDIQMTQTTSSLSASLGDRVTISCRASQDI | a | a | 28. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSVPPTLEVTQQPVRAENQVNVTCQVRKFYPQRLQLTWLE | | | |
| | NGNVSRTETASTVTENKDGTYNWMSWLLVNVSAHRDDVKLTCQVEH | | | |
| | DGQPAVSKSHDLKVSGGGGSFWVLVVVGGVLACYSLLVTVAFIIFWVR | | | |
| | SKRSRLLHSDYMNMTPRRPGPTRKHYQPYAPPRDFAAYRSRVKFSRSA | | | |
| | DAPAYQQGQNQLYNELNLGRREEYDVLDKRRGRDPEMGGKPRRKNP | | | |
| | QEGLYNELQKDKMAEAYSEIGMKGERRRGKGHDGLYQGLSTATKDTY | | | |
| | DALHMQALPPR | | | |
| CAR X1S | MEFGLSWLFLVAILKGVQCSRDIQMTQTTSSLSASLGDRVTISCRASQDI | a | a | 29. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSAKPSAPVVSGPAARATPQHTVSFTCESHGFSPRDITLKW | | | |
| | FKNGNELSDFQTNVDPVGESVSYSIHSTAKVVLTREDVHSQVICEVAHV | | | |
| | TLQGDPLRGTANLSGGGGSKGKHLCPSPLFPGPSKPFWVLVVVGGVL | | | |
| | ACYSLLVTVAFIIFWVRSKRSRLLHSDYMNMTPRRPGPTRKHYQPYAPP | | | |
| | RDFAAYRSRVKFSRSADAPAYQQGQNQLYNELNLGRREEYDVLDKRR | | | |
| | GRDPEMGGKPRRKNPQEGLYNELQKDKMAEAYSEIGMKGERRRGKGH | | | |
| | DGLYQGLSTATKDTYDALHMQALPPR | | | |
| CAR X2S | MEFGLSWLFLVAILKGVQCSRDIQMTQTTSSLSASLGDRVTISCRASQDI | a | a | 30. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSVPPTLEVTQQPVRAENQVNVTCQVRKFYPQRLQLTWLE | | | |
| | NGNVSRTETASTVTENKDGTYNWMSWLLVNVSAHRDDVKLTCQVEH | | | |
| | DGQPAVSKSHDLKVSKGKHLCPSPLFPGPSKPFWVLVVVGGVLACYSL | | | |
| | LVTVAFIIFWVRSKRSRLLHSDYMNMTPRRPGPTRKHYQPYAPPRDFA | | | |
| | AYRSRVKFSRSADAPAYQQGQNQLYNELNLGRREEYDVLDKRRGRDPE | | | |
| | MGGKPRRKNPQEGLYNELQKDKMAEAYSEIGMKGERRRGKGHDGLY | | | |
| | QGLSTATKDTYDALHMQALPPR | | | |
| CAR M | MEFGLSWLFLVAILKGVQCSRDIQMTQTTSSLSASLGDRVTISCRASQDI | a | a | 31. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSAKPSAPVVSGPAARATPQHTVSFTCESHGFSPRDITLKW | | | |
| | FKNGNELSDFQTNVDPVGESVSYSIHSTAKVVLTREDVHSQVICEVAHV | | | |
| | TLQGDPLRGTANLSETIRVPPTLEVTQQPVRAENQVNVTCQVRKFYPQ | | | |
| | RLQLTWLENGNVSRTETASTVTENKDGTYNWMSWLLVNVSAHRDDV | | | |
| | KLTCQVEHDGQPAVSKSHDLKVSGGGGSFWVLVVVGGVLACYSLLVT | | | |
| | VAFIIFWVRSKRSRLLHSDYMNMTPRRPGPTRKHYQPYAPPRDFAAYR | | | |
| | SRVKFSRSADAPAYQQGQNQLYNELNLGRREEYDVLDKRRGRDPEMG | | | |
| | GKPRRKNPQEGLYNELQKDKMAEAYSEIGMKGERRRGKGHDGLYQGL | | | |
| | STATKDTYDALHMQALPPR | | | |
| CAR XM | MEFGLSWLFLVAILKGVQCSRDIQMTQTTSSLSASLGDRVTISCRASQDI | a | a | 32. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSAKPSAPVVSGPAARATPQHTVSFTCESHGFSPRDITLKW | | | |
| | FKNGNELSDFQTNVDPVGESVSYSIHSTAKVVLTREDVHSQVICEVAHV | | | |
| | TLQGDPLRGTANLSETIRVPPTLEVTQQPVRAENQVNVTCQVRKFYPQ | | | |
| | RLQLTWLENGNVSRTETASTVTENKDGTYNWMSWLLVNVSAHRDDV | | | |
| | KLTCQVEHDGQPAVSKSHDLKVSKGKHLCPSPLFPGPSKPFWVLVVVG | | | |
| | GVLACYSLLVTVAFIIFWVRSKRSRLLHSDYMNMTPRRPGPTRKHYQPY | | | |
| | APPRDFAAYRSRVKFSRSADAPAYQQGQNQLYNELNLGRREEYDVLDK | | | |
| | RRGRDPEMGGKPRRKNPQEGLYNELQKDKMAEAYSEIGMKGERRRGK | | | |
| | GHDGLYQGLSTATKDTYDALHMQALPPR | | | |
| CAR L | MEFGLSWLFLVAILKGVQCSRDIQMTQTTSSLSASLGDRVTISCRASQDI | a | a | 33. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSVPPTLEVTQQPVRAENQVNVTCQVRKFYPQRLQLTWLE | | | |
| | NGNVSRTETASTVTENKDGTYNWMSWLLVNVSAHRDDVKLTCQVEH | | | |
| | DGQPAVSKSHDLKVSGGGGSAKPSAPVVSGPAARATPQHTVSFTCESH | | | |
| | GFSPRDITLKWFKNGNELSDFQTNVDPVGESVSYSIHSTAKVVLTREDV | | | |
| | HSQVICEVAHVTLQGDPLRGTANLSETIRVPPTLEVTQQPVRAENQVN | | | |
| | VTCQVRKFYPQRLQLTWLENGNVSRTETASTVTENKDGTYNWMSWL | | | |
| | LVNVSAHRDDVKLTCQVEHDGQPAVSKSHDLKVSGGGGSFWVLVVVG | | | |
| | GVLACYSLLVTVAFIIFWVRSKRSRLLHSDYMNMTPRRPGPTRKHYQPY | | | |
| | APPRDFAAYRSRVKFSRSADAPAYQQGQNQLYNELNLGRREEYDVLDK | | | |
| | RRGRDPEMGGKPRRKNPQEGLYNELQKDKMAEAYSEIGMKGERRRGK | | | |
| | GHDGLYQGLSTATKDTYDALHMQALPPR | | | |
| CAR XL | MEFGLSWLFLVAILKGVQCSRDIQMTQTTSSLSASLGDRVTISCRASQDI | a | a | 34. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSYVTVSSQDPAEPKSPDKTHTC | | | |
| | PPCPGGGGSVPPTLEVTQQPVRAENQVNVTCQVRKFYPQRLQLTWLE | | | |
| | NGNVSRTETASTVTENKDGTYNWMSWLLVNVSAHRDDVKLTCQVEH | | | |
| | DGQPAVSKSHDLKVSGGGGSAKPSAPVVSGPAARATPQHTVSFTCESH | | | |
| | GFSPRDITLKWFKNGNELSDFQTNVDPVGESVSYSIHSTAKVVLTREDV | | | |
| | HSQVICEVAHVTLQGDPLRGTANLSETIRVPPTLEVTQQPVRAENQVN | | | |
| | VTCQVRKFYPQRLQLTWLENGNVSRTETASTVTENKDGTYNWMSWL | | | |
| | LVNVSAHRDDVKLTCQVEHDGQPAVSKSHDLKVSKGKHLCPSPLFPGP | | | |
| | SKPFWVLVVVGGVLACYSLLVTVAFIIFWVRSKRSRLLHSDYMNMTPRR | | | |
| | PGPTRKHYQPYAPPRDFAAYRSRVKFSRSADAPAYQQGQNQLYNELN | | | |
| | LGRREEYDVLDKRRGRDPEMGGKPRRKNPQEGLYNELQKDKMAEAYS | | | |
| | EIGMKGERRRGKGHDGLYQGLSTATKDTYDALHMQALPPR | | | |
| CAR spacer XS | AGCTACGTGACCGTGAGCAGCCAGGACCCCGCCGAGCCCAAGAGC | n | a | 35. |
| | CCCGACAAGACCCACACCTGCCCCCCCTGCCCCAAGGGCAAGCAC | | | |
| | CTGTGCCCCAGCCCCCTGTTCCCCGGCCCCAGCAAGCCC | | | |
| CAR spacer 1S | AGCTACGTGACCGTGAGCAGCCAGGACCCCGCCGAGCCCAAGAGC | n | a | 36. |
| | CCCGACAAGACCCACACCTGCCCCCCCTGCCCCGGAGGAGGAGGA | | | |
| | TCTGCCAAGCCCAGCGCCCCCGTGGTGAGCGGCCCCGCCGCCAG | | | |
| | GGCCACCCCCCAGCACACCGTGAGCTTCACCTGCGAGAGCCACGG | | | |
| | CTTCAGCCCCAGGGACATCACCCTGAAGTGGTTCAAGAACGGCAA | | | |
| | CGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGC | | | |
| CAR spacer 25 | AGCTACGTGACCGTGAGCAGCCAGGACCCCGCCGAGCCCAAGAGC | n | a | 37. |
| | CCCGACAAGACCCACACCTGCCCCCCCTGCCCCGGAGGAGGAGGA | | | |
| | TCTGTGCCCCCCACCCTGGAGGTGACCCAGCAGCCCGTGAGGGCC | | | |
| | GAGAACCAGGTGAACGTGACCTGCCAGGTGAGGAAGTTCTACCCC | | | |
| | CAGAGGCTGCAGCTGACCTGGCTGGAGAACGGCAACGTGAGCAG | | | |
| | GACCGAGACCGCCAGCACCGTGACCGAGAACAAGGACGGCACCTA | | | |
| | CAACTGGATGAGCTGGCTGCTGGTGAACGTGAGCGCCCACAGGG | | | |
| | ACGACGTGAAGCTGACCTGCCAGGTGGAGCACGACGGCCAGCCC | | | |
| | GCCGTGAGCAAGAGCCACGACCTGAAGGTGAGC | | | |
| CAR spacer | AGCTACGTGACCGTGAGCAGCCAGGACCCCGCCGAGCCCAAGAGC | n | a | 38. |
| X1S | CCCGACAAGACCCACACCTGCCCCCCCTGCCCCGGAGGAGGAGGA | | | |
| | TCTGCCAAGCCCAGCGCCCCCGTGGTGAGCGGCCCCGCCGCCAG | | | |
| | GGCCACCCCCCAGCACACCGTGAGCTTCACCTGCGAGAGCCACGG | | | |
| | CTTCAGCCCCAGGGACATCACCCTGAAGTGGTTCAAGAACGGCAA | | | |
| | CGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGGA | | | |
| | GGAGGAGGATCTAAGGGCAAGCACCTGTGCCCCAGCCCCCTGTTC | | | |
| | CCCGGCCCCAGCAAGCCC | | | |
| CAR spacer | AGCTACGTGACCGTGAGCAGCCAGGACCCCGCCGAGCCCAAGAGC | n | a | 39. |
| X2S | CCCGACAAGACCCACACCTGCCCCCCCTGCCCCGGAGGAGGAGGA | | | |
| | TCTGTGCCCCCCACCCTGGAGGTGACCCAGCAGCCCGTGAGGGCC | | | |
| | GAGAACCAGGTGAACGTGACCTGCCAGGTGAGGAAGTTCTACCCC | | | |
| | CAGAGGCTGCAGCTGACCTGGCTGGAGAACGGCAACGTGAGCAG | | | |
| | GACCGAGACCGCCAGCACCGTGACCGAGAACAAGGACGGCACCTA | | | |
| | CAACTGGATGAGCTGGCTGCTGGTGAACGTGAGCGCCCACAGGG | | | |
| | ACGACGTGAAGCTGACCTGCCAGGTGGAGCACGACGGCCAGCCC | | | |
| | GCCGTGAGCAAGAGCCACGACCTGAAGGTGAGCAAGGGCAAGCA | | | |
| | CCTGTGCCCCAGCCCCCTGTTCCCCGGCCCCAGCAAGCCC | | | |
| CAR spacer M | AGCTACGTGACCGTGAGCAGCCAGGACCCCGCCGAGCCCAAGAGC | n | a | 40. |
| | CCCGACAAGACCCACACCTGCCCCCCCTGCCCCGGAGGAGGAGGA | | | |
| | TCTGCCAAGCCCAGCGCCCCCGTGGTGAGCGGCCCCGCCGCCAG | | | |
| | GGCCACCCCCCAGCACACCGTGAGCTTCACCTGCGAGAGCCACGG | | | |
| | CTTCAGCCCCAGGGACATCACCCTGAAGTGGTTCAAGAACGGCAA | | | |
| | CGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGAG | | | |
| | ACCATCAGGGTCCCCCCACCCTGGAGGTGACCCAGCAGCCCGTGA | | | |
| | GGGCCGAGAACCAGGTGAACGTGACCTGCCAGGTGAGGAAGTTC | | | |
| | TACCCCCAGAGGCTGCAGCTGACCTGGCTGGAGAACGGCAACGTG | | | |
| | AGCAGGACCGAGACCGCCAGCACCGTGACCGAGAACAAGGACGGC | | | |
| | ACCTACAACTGGATGAGCTGGCTGCTGGTGAACGTGAGCGCCCAC | | | |
| | AGGGACGACGTGAAGCTGACCTGCCAGGTGGAGCACGACGGCCA | | | |
| | GCCCGCCGTGAGCAAGAGCCACGACCTGAAGGTGAGC | | | |
| CAR spacer XM | AGCTACGTGACCGTGAGCAGCCAGGACCCCGCCGAGCCCAAGAGC | n | a | 41. |
| | CCCGACAAGACCCACACCTGCCCCCCCTGCCCCGGAGGAGGAGGA | | | |
| | TCTGCCAAGCCCAGCGCCCCCGTGGTGAGCGGCCCCGCCGCCAG | | | |
| | GGCCACCCCCCAGCACACCGTGAGCTTCACCTGCGAGAGCCACGG | | | |
| | CTTCAGCCCCAGGGACATCACCCTGAAGTGGTTCAAGAACGGCAA | | | |
| | CGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGAG | | | |
| | ACCATCAGGGTGCCCCCCACCCTGGAGGTGACCCAGCAGCCCGTG | | | |
| | AGGGCCGAGAACCAGGTGAACGTGACCTGCCAGGTGAGGAAGTT | | | |
| | CTACCCCCAGAGGCTGCAGCTGACCTGGCTGGAGAACGGCAACGT | | | |
| | GAGCAGGACCGAGACCGCCAGCACCGTGACCGAGAACAAGGACG | | | |
| | GCACCTACAACTGGATGAGCTGGCTGCTGGTGAACGTGAGCGCCC | | | |
| | ACAGGGACGACGTGAAGCTGACCTGCCAGGTGGAGCACGACGGC | | | |
| | CAGCCCGCCGTGAGCAAGAGCCACGACCTGAAGGTGAGCAAGGG | | | |
| | CAAGCACCTGTGCCCCAGCCCCCTGTTCCCCGGCCCCAGCAAGCC | | | |
| | C | | | |
| CAR spacer L | AGCTACGTGACCGTGAGCAGCCAGGACCCCGCCGAGCCCAAGAGC | n | a | 42. |
| | CCCGACAAGACCCACACCTGCCCCCCCTGCCCCGGTGGCGGTGGA | | | |
| | AGTGTGCCCCCCACCCTGGAGGTGACCCAGCAGCCCGTGAGAGCC | | | |
| | GAGAACCAGGTGAACGTGACCTGCCAGGTGAGAAAGTTCTACCCC | | | |
| | CAGAGACTGCAGCTGACCTGGCTGGAGAACGGCAACGTGAGCAGA | | | |
| | ACCGAGACCGCCAGCACCGTGACCGAGAACAAGGACGGCACCTAC | | | |
| | AACTGGATGAGCTGGCTGCTGGTGAACGTGAGCGCCCACAGAGAC | | | |
| | GACGTGAAGCTGACCTGCCAGGTGGAGCACGACGGCCAGCCCGC | | | |
| | CGTGAGCAAGAGCCACGACCTGAAGGTGAGCGGCGGAGGCGGGA | | | |
| | GTGCCAAGCCCAGCGCCCCCGTGGTGAGCGGCCCCGCCGCCAGG | | | |
| | GCCACCCCCCAGCACACCGTGAGCTTCACCTGCGAGAGCCACGGC | | | |
| | TTCAGCCCCAGGGACATCACCCTGAAGTGGTTCAAGAACGGCAAC | | | |
| | GAGCTGAGCGACTTCCAGACCAACGTGGACCCCGTGGGCGAGAG | | | |
| | CGTGAGCTACAGCATCCACAGCACCGCCAAGGTGGTGCTGACCAG | | | |
| | GGAGGACGTGCACAGCCAGGTGATCTGCGAGGTGGCCCACGTGA | | | |
| | CCCTGCAGGGCGACCCCCTGAGGGGCACCGCCAACCTGAGCGAGA | | | |
| | CCATCAGGGTGCCCCCCACCCTGGAGGTGACCCAGCAGCCCGTGA | | | |
| | GGGCCGAGAACCAGGTGAACGTGACCTGCCAGGTGAGGAAGTTC | | | |
| | TACCCCCAGAGGCTGCAGCTGACCTGGCTGGAGAACGGCAACGTG | | | |
| | AGCAGGACCGAGACCGCCAGCACCGTGACCGAGAACAAGGACGGC | | | |
| | ACCTACAACTGGATGAGCTGGCTGCTGGTGAACGTGAGCGCCCAC | | | |
| | AGGGACGACGTGAAGCTGACCTGCCAGGTGGAGCACGACGGCCA | | | |
| | GCCCGCCGTGAGCAAGAGCCACGACCTGAAGGTGAGC | | | |
| CAR spacer XL | AGCTACGTGACCGTGAGCAGCCAGGACCCCGCCGAGCCCAAGAGC | n | a | 43. |
| | CCCGACAAGACCCACACCTGCCCCCCCTGCCCCGGTGGCGGTGGA | | | |
| | AGTGTGCCCCCCACCCTGGAGGTGACCCAGCAGCCCGTGAGAGCC | | | |
| | GAGAACCAGGTGAACGTGACCTGCCAGGTGAGAAAGTTCTACCCC | | | |
| | CAGAGACTGCAGCTGACCTGGCTGGAGAACGGCAACGTGAGCAGA | | | |
| | ACCGAGACCGCCAGCACCGTGACCGAGAACAAGGACGGCACCTAC | | | |
| | AACTGGATGAGCTGGCTGCTGGTGAACGTGAGCGCCCACAGAGAC | | | |
| | GACGTGAAGCTGACCTGCCAGGTGGAGCACGACGGCCAGCCCGC | | | |
| | CGTGAGCAAGAGCCACGACCTGAAGGTGAGCGGCGGAGGCGGGA | | | |
| | GTGCCAAGCCCAGCGCCCCCGTGGTGAGCGGCCCCGCCGCCAGG | | | |
| | GCCACCCCCCAGCACACCGTGAGCTTCACCTGCGAGAGCCACGGC | | | |
| | TTCAGCCCCAGGGACATCACCCTGAAGTGGTTCAAGAACGGCAAC | | | |
| | GAGCTGAGCGACTTCCAGACCAACGTGGACCCCGTGGGCGAGAG | | | |
| | CGTGAGCTACAGCATCCACAGCACCGCCAAGGTGGTGCTGACCAG | | | |
| | GGAGGACGTGCACAGCCAGGTGATCTGCGAGGTGGCCCACGTGA | | | |
| | CCCTGCAGGGCGACCCCCTGAGGGGCACCGCCAACCTGAGCGAGA | | | |
| | CCATCAGGGTGCCCCCCACCCTGGAGGTGACCCAGCAGCCCGTGA | | | |
| | GGGCCGAGAACCAGGTGAACGTGACCTGCCAGGTGAGGAAGTTC | | | |
| | TACCCCCAGAGGCTGCAGCTGACCTGGCTGGAGAACGGCAACGTG | | | |
| | AGCAGGACCGAGACCGCCAGCACCGTGACCGAGAACAAGGACGGC | | | |
| | ACCTACAACTGGATGAGCTGGCTGCTGGTGAACGTGAGCGCCCAC | | | |
| | AGGGACGACGTGAAGCTGACCTGCCAGGTGGAGCACGACGGCCA | | | |
| | GCCCGCCGTGAGCAAGAGCCACGACCTGAAGGTGAGCAAGGGCA | | | |
| | AGCACCTGTGCCCCAGCCCCCTGTTCCCCGGCCCCAGCAAGCCC | | | |
| CAR XS | ATGGAGTTCGGCCTGAGCTGGCTGTTCCTGGTGGCCATCCTGAAG | n | a | 44. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGTGGTGGTGGTTCTG | | | |
| | GTGGTGGTGGTTCTGGCGGCGGCGGCTCCGGTGGTGGTGGGTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCAAGGGCAAGC | | | |
| | ACCTGTGCCCCAGCCCCCTGTTCCCCGGCCCCAGCAAGCCCTTCT | | | |
| | GGGTGCTGGTGGTGGTGGGCGGCGTGCTGGCCTGCTACAGCCTG | | | |
| | CTGGTGACCGTGGCCTTCATCATCTTCTGGGTGAGGAGCAAGAGG | | | |
| | AGCAGGCTGCTGCACAGCGACTACATGAACATGACCCCCAGGAGG | | | |
| | CCCGGCCCCACCAGGAAGCACTACCAGCCCTACGCCCCCCCCAGG | | | |
| | GACTTCGCCGCCTACAGGAGCAGGGTGAAGTTCAGCAGGAGCGC | | | |
| | CGACGCCCCCGCCTACCAGCAGGGCCAGAACCAGCTGTACAACGA | | | |
| | GCTGAACCTGGGCAGGAGGGAGGAGTACGACGTGCTGGACAAGA | | | |
| | GGAGGGGCAGGGACCCCGAGATGGGCGGCAAGCCCAGGAGGAAG | | | |
| | AACCCCCAGGAGGGCCTGTACAACGAGCTGCAGAAGGACAAGATG | | | |
| | GCCGAGGCCTACAGCGAGATCGGCATGAAGGGCGAGAGGAGGAG | | | |
| | GGGCAAGGGCCACGACGGCCTGTACCAGGGCCTGAGCACCGCCA | | | |
| | CCAAGGACACCTACGACGCCCTGCACATGCAGGCCCTGCCCCCCA | | | |
| | GGTAA | | | |
| CAR 1S | ATGGAGTTCGGCCTGAGCTGGCTGTTCCTGGTGGCCATCCTGAAG | n | a | 45. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGTGGTGGTGGTTCTG | | | |
| | GTGGTGGTGGTTCTGGCGGCGGCGGCTCCGGTGGTGGTGGGTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGAGGAGGAG | | | |
| | GATCTGCCAAGCCCAGCGCCCCCGTGGTGAGCGGCCCCGCCGCCA | | | |
| | GGGCCACCCCCCAGCACACCGTGAGCTTCACCTGCGAGAGCCACG | | | |
| | GCTTCAGCCCCAGGGACATCACCCTGAAGTGGTTCAAGAACGGCA | | | |
| | ACGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGGT | | | |
| | GGTGGTGGTTCCTTCTGGGTGCTGGTGGTGGTGGGCGGCGTGCT | | | |
| | GGCCTGCTACAGCCTGCTGGTGACCGTGGCCTTCATCATCTTCTG | | | |
| | GGTGAGGAGCAAGAGGAGCAGGCTGCTGCACAGCGACTACATGA | | | |
| | ACATGACCCCCAGGAGGCCCGGCCCCACCAGGAAGCACTACCAGC | | | |
| | CCTACGCCCCCCCCAGGGACTTCGCCGCCTACAGGAGCAGGGTGA | | | |
| | AGTTCAGCAGGAGCGCCGACGCCCCCGCCTACCAGCAGGGCCAGA | | | |
| | ACCAGCTGTACAACGAGCTGAACCTGGGCAGGAGGGAGGAGTAC | | | |
| | GACGTGCTGGACAAGAGGAGGGGCAGGGACCCCGAGATGGGCGG | | | |
| | CAAGCCCAGGAGGAAGAACCCCCAGGAGGGCCTGTACAACGAGCT | | | |
| | GCAGAAGGACAAGATGGCCGAGGCCTACAGCGAGATCGGCATGAA | | | |
| | GGGCGAGAGGAGGAGGGGCAAGGGCCACGACGGCCTGTACCAGG | | | |
| | GCCTGAGCACCGCCACCAAGGACACCTACGACGCCCTGCACATGC | | | |
| | AGGCCCTGCCCCCCAGGTAA | | | |
| CAR 2S | ATGGAGTTCGGCCTGAGCTGGCTGTTCCTGGTGGCCATCCTGAAG | n | a | 46. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGTGGTGGTGGTTCTG | | | |
| | GTGGTGGTGGTTCTGGCGGCGGCGGCTCCGGTGGTGGTGGGTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGAGGAGGAG | | | |
| | GATCTGTGCCCCCCACCCTGGAGGTGACCCAGCAGCCCGTGAGGG | | | 47. |
| | CCGAGAACCAGGTGAACGTGACCTGCCAGGTGAGGAAGTTCTACC | | | |
| | CCCAGAGGCTGCAGCTGACCTGGCTGGAGAACGGCAACGTGAGC | | | |
| | AGGACCGAGACCGCCAGCACCGTGACCGAGAACAAGGACGGCACC | | | |
| | TACAACTGGATGAGCTGGCTGCTGGTGAACGTGAGCGCCCACAGG | | | |
| | GACGACGTGAAGCTGACCTGCCAGGTGGAGCACGACGGCCAGCC | | | |
| | CGCCGTGAGCAAGAGCCACGACCTGAAGGTGAGCGGTGGTGGTG | | | |
| | GTTCCTTCTGGGTGCTGGTGGTGGTGGGCGGCGTGCTGGCCTGC | | | |
| | TACAGCCTGCTGGTGACCGTGGCCTTCATCATCTTCTGGGTGAGG | | | |
| | AGCAAGAGGAGCAGGCTGCTGCACAGCGACTACATGAACATGACC | | | |
| | CCCAGGAGGCCCGGCCCCACCAGGAAGCACTACCAGCCCTACGCC | | | |
| | CCCCCCAGGGACTTCGCCGCCTACAGGAGCAGGGTGAAGTTCAGC | | | |
| | AGGAGCGCCGACGCCCCCGCCTACCAGCAGGGCCAGAACCAGCTG | | | |
| | TACAACGAGCTGAACCTGGGCAGGAGGGAGGAGTACGACGTGCT | | | |
| | GGACAAGAGGAGGGGCAGGGACCCCGAGATGGGGGGCAAGCCCA | | | |
| | GGAGGAAGAACCCCCAGGAGGGCCTGTACAACGAGCTGCAGAAG | | | |
| | GACAAGATGGCCGAGGCCTACAGCGAGATCGGCATGAAGGGCGA | | | |
| | GAGGAGGAGGGGCAAGGGCCACGACGGCCTGTACCAGGGCCTGA | | | |
| | GCACCGCCACCAAGGACACCTACGACGCCCTGCACATGCAGGCCC | | | |
| | TGCCCCCCAGGTAA | | | |
| CAR X1S | ATGGAGTTCGGCCTGAGCTGGCTGTTCCTGGTGGCCATCCTGAAG | | | 47. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGTGGTGGTGGTTCTG | | | |
| | GTGGTGGTGGTTCTGGCGGCGGCGGCTCCGGTGGTGGTGGGTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGAGGAGGAG | | | |
| | GATCTGCCAAGCCCAGCGCCCCCGTGGTGAGCGGCCCCGCCGCCA | | | |
| | GGGCCACCCCCCAGCACACCGTGAGCTTCACCTGCGAGAGCCACG | | | |
| | GCTTCAGCCCCAGGGACATCACCCTGAAGTGGTTCAAGAACGGCA | | | |
| | ACGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGGA | | | |
| | GGAGGAGGATCTAAGGGCAAGCACCTGTGCCCCAGCCCCCTGTTC | | | |
| | CCCGGCCCCAGCAAGCCCTTCTGGGTGCTGGTGGTGGTGGGCGG | | | |
| | CGTGCTGGCCTGCTACAGCCTGCTGGTGACCGTGGCCTTCATCAT | | | |
| | CTTCTGGGTGAGGAGCAAGAGGAGCAGGCTGCTGCACAGCGACT | | | |
| | ACATGAACATGACCCCCAGGAGGCCCGGCCCCACCAGGAAGCACT | | | |
| | ACCAGCCCTACGCCCCCCCCAGGGACTTCGCCGCCTACAGGAGCA | | | |
| | GGGTGAAGTTCAGCAGGAGCGCCGACGCCCCCGCCTACCAGCAG | | | |
| | GGCCAGAACCAGCTGTACAACGAGCTGAACCTGGGCAGGAGGGA | | | |
| | GGAGTACGACGTGCTGGACAAGAGGAGGGGCAGGGACCCCGAGA | | | |
| | TGGGCGGCAAGCCCAGGAGGAAGAACCCCCAGGAGGGCCTGTAC | | | |
| | AACGAGCTGCAGAAGGACAAGATGGCCGAGGCCTACAGCGAGATC | | | |
| | GGCATGAAGGGCGAGAGGAGGAGGGGCAAGGGCCACGACGGCCT | | | |
| | GTACCAGGGCCTGAGCACCGCCACCAAGGACACCTACGACGCCCT | | | |
| | GCACATGCAGGCCCTGCCCCCCAGGTAA | | | |
| CAR X2S | ATGGAGTTCGGCCTGAGCTGGCTGTTCCTGGTGGCCATCCTGAAG | n | a | 48. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGTGGTGGTGGTTCTG | | | |
| | GTGGTGGTGGTTCTGGCGGCGGCGGCTCCGGTGGTGGTGGGTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGAGGAGGAG | | | |
| | GATCTGTGCCCCCCACCCTGGAGGTGACCCAGCAGCCCGTGAGGG | | | |
| | CCGAGAACCAGGTGAACGTGACCTGCCAGGTGAGGAAGTTCTACC | | | |
| | CCCAGAGGCTGCAGCTGACCTGGCTGGAGAACGGCAACGTGAGC | | | |
| | AGGACCGAGACCGCCAGCACCGTGACCGAGAACAAGGACGGCACC | | | |
| | TACAACTGGATGAGCTGGCTGCTGGTGAACGTGAGCGCCCACAGG | | | |
| | GACGACGTGAAGCTGACCTGCCAGGTGGAGCACGACGGCCAGCC | | | |
| | CGCCGTGAGCAAGAGCCACGACCTGAAGGTGAGCAAGGGCAAGC | | | |
| | ACCTGTGCCCCAGCCCCCTGTTCCCCGGCCCCAGCAAGCCCTTCT | | | |
| | GGGTGCTGGTGGTGGTGGGCGGCGTGCTGGCCTGCTACAGCCTG | | | |
| | CTGGTGACCGTGGCCTTCATCATCTTCTGGGTGAGGAGCAAGAGG | | | |
| | AGCAGGCTGCTGCACAGCGACTACATGAACATGACCCCCAGGAGG | | | |
| | CCCGGCCCCACCAGGAAGCACTACCAGCCCTACGCCCCCCCCAGG | | | |
| | GACTTCGCCGCCTACAGGAGCAGGGTGAAGTTCAGCAGGAGCGC | | | |
| | CGACGCCCCCGCCTACCAGCAGGGCCAGAACCAGCTGTACAACGA | | | |
| | GCTGAACCTGGGCAGGAGGGAGGAGTACGACGTGCTGGACAAGA | | | |
| | GGAGGGGCAGGGACCCCGAGATGGGCGGCAAGCCCAGGAGGAAG | | | |
| | AACCCCCAGGAGGGCCTGTACAACGAGCTGCAGAAGGACAAGATG | | | |
| | GCCGAGGCCTACAGCGAGATCGGCATGAAGGGCGAGAGGAGGAG | | | |
| | GGGCAAGGGCCACGACGGCCTGTACCAGGGCCTGAGCACCGCCA | | | |
| | CCAAGGACACCTACGACGCCCTGCACATGCAGGCCCTGCCCCCCA | | | |
| | GGTAA | | | |
| CAR M | ATGGAGTTCGGCCTGAGCTGGCTGTTCCTGGTGGCCATCCTGAAG | n | a | 49. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGTGGTGGTGGTTCTG | | | |
| | GTGGTGGTGGTTCTGGCGGCGGCGGCTCCGGTGGTGGTGGGTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGAGGAGGAG | | | |
| | GATCTGCCAAGCCCAGCGCCCCCGTGGTGAGCGGCCCCGCCGCCA | | | |
| | GGGCCACCCCCCAGCACACCGTGAGCTTCACCTGCGAGAGCCACG | | | |
| | GCTTCAGCCCCAGGGACATCACCCTGAAGTGGTTCAAGAACGGCA | | | |
| | ACGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGAG | | | |
| | ACCATCAGGGTGCCCCCCACCCTGGAGGTGACCCAGCAGCCCGTG | | | |
| | AGGGCCGAGAACCAGGTGAACGTGACCTGCCAGGTGAGGAAGTT | | | |
| | CTACCCCCAGAGGCTGCAGCTGACCTGGCTGGAGAACGGCAACGT | | | |
| | GAGCAGGACCGAGACCGCCAGCACCGTGACCGAGAACAAGGACG | | | |
| | GCACCTACAACTGGATGAGCTGGCTGCTGGTGAACGTGAGCGCCC | | | |
| | ACAGGGACGACGTGAAGCTGACCTGCCAGGTGGAGCACGACGGC | | | |
| | CAGCCCGCCGTGAGCAAGAGCCACGACCTGAAGGTGAGCGGTGG | | | |
| | TGGTGGTTCCTTCTGGGTGCTGGTGGTGGTGGGCGGCGTGCTGG | | | |
| | CCTGCTACAGCCTGCTGGTGACCGTGGCCTTCATCATCTTCTGGG | | | |
| | TGAGGAGCAAGAGGAGCAGGCTGCTGCACAGCGACTACATGAACA | | | |
| | TGACCCCCAGGAGGCCCGGCCCCACCAGGAAGCACTACCAGCCCT | | | |
| | ACGCCCCCCCCAGGGACTTCGCCGCCTACAGGAGCAGGGTGAAGT | | | |
| | TCAGCAGGAGCGCCGACGCCCCCGCCTACCAGCAGGGCCAGAACC | | | |
| | AGCTGTACAACGAGCTGAACCTGGGCAGGAGGGAGGAGTACGAC | | | |
| | GTGCTGGACAAGAGGAGGGGCAGGGACCCCGAGATGGGCGGCAA | | | |
| | GCCCAGGAGGAAGAACCCCCAGGAGGGCCTGTACAACGAGCTGCA | | | |
| | GAAGGACAAGATGGCCGAGGCCTACAGCGAGATCGGCATGAAGG | | | |
| | GCGAGAGGAGGAGGGGCAAGGGCCACGACGGCCTGTACCAGGGC | | | |
| | CTGAGCACCGCCACCAAGGACACCTACGACGCCCTGCACATGCAG | | | |
| | GCCCTGCCCCCCAGGTAA | | | |
| CAR XM | ATGGAGTTCGGCCTGAGCTGGCTGTTCCTGGTGGCCATCCTGAAG | n | a | 50. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGTGGTGGTGGTTCTG | | | |
| | GTGGTGGTGGTTCTGGCGGCGGCGGCTCCGGTGGTGGTGGGTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGAGGAGGAG | | | |
| | GATCTGCCAAGCCCAGCGCCCCCGTGGTGAGCGGCCCCGCCGCCA | | | |
| | GGGCCACCCCCCAGCACACCGTGAGCTTCACCTGCGAGAGCCACG | | | |
| | GCTTCAGCCCCAGGGACATCACCCTGAAGTGGTTCAAGAACGGCA | | | |
| | ACGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGAG | | | |
| | ACCATCAGGGTGCCCCCCACCCTGGAGGTGACCCAGCAGCCCGTG | | | |
| | AGGGCCGAGAACCAGGTGAACGTGACCTGCCAGGTGAGGAAGTT | | | |
| | CTACCCCCAGAGGCTGCAGCTGACCTGGCTGGAGAACGGCAACGT | | | |
| | GAGCAGGACCGAGACCGCCAGCACCGTGACCGAGAACAAGGACG | | | |
| | GCACCTACAACTGGATGAGCTGGCTGCTGGTGAACGTGAGCGCCC | | | |
| | ACAGGGACGACGTGAAGCTGACCTGCCAGGTGGAGCACGACGGC | | | |
| | CAGCCCGCCGTGAGCAAGAGCCACGACCTGAAGGTGAGCAAGGG | | | |
| | CAAGCACCTGTGCCCCAGCCCCCTGTTCCCCGGCCCCAGCAAGCC | | | |
| | CTTCTGGGTGCTGGTGGTGGTGGGCGGCGTGCTGGCCTGCTACA | | | |
| | GCCTGCTGGTGACCGTGGCCTTCATCATCTTCTGGGTGAGGAGCA | | | |
| | AGAGGAGCAGGCTGCTGCACAGCGACTACATGAACATGACCCCCA | | | |
| | GGAGGCCCGGCCCCACCAGGAAGCACTACCAGCCCTACGCCCCCC | | | |
| | CCAGGGACTTCGCCGCCTACAGGAGCAGGGTGAAGTTCAGCAGGA | | | |
| | GCGCCGACGCCCCCGCCTACCAGCAGGGCCAGAACCAGCTGTACA | | | |
| | ACGAGCTGAACCTGGGCAGGAGGGAGGAGTACGACGTGCTGGAC | | | |
| | AAGAGGAGGGGCAGGGACCCCGAGATGGGCGGCAAGCCCAGGAG | | | |
| | GAAGAACCCCCAGGAGGGCCTGTACAACGAGCTGCAGAAGGACAA | | | |
| | GATGGCCGAGGCCTACAGCGAGATCGGCATGAAGGGCGAGAGGA | | | |
| | GGAGGGGCAAGGGCCACGACGGCCTGTACCAGGGCCTGAGCACC | | | |
| | GCCACCAAGGACACCTACGACGCCCTGCACATGCAGGCCCTGCCC | | | |
| | CCCAGGTAA | | | |
| CAR L | ATGGAGTTCGGCCTGAGCTGGCTGTTCCTGGTGGCCATCCTGAAG | n | a | 51. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGCGGTGGAGGTTCCG | | | |
| | GCGGTGGCGGTTCCGGAGGCGGTGGGTCAGGAGGTGGAGGCTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGTGGCGGTG | | | |
| | GAAGTGTGCCCCCCACCCTGGAGGTGACCCAGCAGCCCGTGAGAG | | | |
| | CCGAGAACCAGGTGAACGTGACCTGCCAGGTGAGAAAGTTCTACC | | | |
| | CCCAGAGACTGCAGCTGACCTGGCTGGAGAACGGCAACGTGAGCA | | | |
| | GAACCGAGACCGCCAGCACCGTGACCGAGAACAAGGACGGCACCT | | | |
| | ACAACTGGATGAGCTGGCTGCTGGTGAACGTGAGCGCCCACAGAG | | | |
| | ACGACGTGAAGCTGACCTGCCAGGTGGAGCACGACGGCCAGCCC | | | |
| | GCCGTGAGCAAGAGCCACGACCTGAAGGTGAGCGGCGGAGGCGG | | | |
| | GAGTGCCAAGCCCAGCGCCCCCGTGGTGAGCGGCCCCGCCGCCA | | | |
| | GGGCCACCCCCCAGCACACCGTGAGCTTCACCTGCGAGAGCCACG | | | |
| | GCTTCAGCCCCAGGGACATCACCCTGAAGTGGTTCAAGAACGGCA | | | |
| | ACGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGAG | | | |
| | ACCATCAGGGTGCCCCCCACCCTGGAGGTGACCCAGCAGCCCGTG | | | |
| | AGGGCCGAGAACCAGGTGAACGTGACCTGCCAGGTGAGGAAGTT | | | |
| | CTACCCCCAGAGGCTGCAGCTGACCTGGCTGGAGAACGGCAACGT | | | |
| | GAGCAGGACCGAGACCGCCAGCACCGTGACCGAGAACAAGGACG | | | |
| | GCACCTACAACTGGATGAGCTGGCTGCTGGTGAACGTGAGCGCCC | | | |
| | ACAGGGACGACGTGAAGCTGACCTGCCAGGTGGAGCACGACGGC | | | |
| | CAGCCCGCCGTGAGCAAGAGCCACGACCTGAAGGTGAGCGGCGG | | | |
| | TGGCGGCAGCTTCTGGGTGCTGGTGGTGGTGGGCGGCGTGCTG | | | |
| | GCCTGCTACAGCCTGCTGGTGACCGTGGCCTTCATCATCTTCTGG | | | |
| | GTGAGGAGCAAGAGGAGCAGGCTGCTGCACAGCGACTACATGAAC | | | |
| | ATGACCCCCAGGAGGCCCGGCCCCACCAGGAAGCACTACCAGCCC | | | |
| | TACGCCCCCCCCAGGGACTTCGCCGCCTACAGGAGCAGGGTGAAG | | | |
| | TTCAGCAGGAGCGCCGACGCCCCCGCCTACCAGCAGGGCCAGAAC | | | |
| | CAGCTGTACAACGAGCTGAACCTGGGCAGGAGGGAGGAGTACGA | | | |
| | CGTGCTGGACAAGAGGAGGGGCAGGGACCCCGAGATGGGCGGCA | | | |
| | AGCCCAGGAGGAAGAACCCCCAGGAGGGCCTGTACAACGAGCTGC | | | |
| | AGAAGGACAAGATGGCCGAGGCCTACAGCGAGATCGGCATGAAG | | | |
| | GGCGAGAGGAGGAGGGGCAAGGGCCACGACGGCCTGTACCAGGG | | | |
| | CCTGAGCACCGCCACCAAGGACACCTACGACGCCCTGCACATGCA | | | |
| | GGCCCTGCCCCCCAGGTAA | | | |
| CAR XL | ATGGAGTTCGGCCTGAGCTGGCTGTTCCTGGTGGCCATCCTGAAG | n | a | 52. |
| | GGCGTGCAGTGCAGCAGGGACATCCAGATGACCCAGACCACCAGC | | | |
| | AGCCTGAGCGCCAGCCTGGGCGACAGGGTGACCATCAGCTGCAG | | | |
| | GGCCAGCCAGGACATCAGCAAGTACCTGAACTGGTACCAGCAGAA | | | |
| | GCCCGACGGCACCGTGAAGCTGCTGATCTACCACACCAGCAGGCT | | | |
| | GCACAGCGGCGTGCCCAGCAGGTTCAGCGGCAGCGGCAGCGGCA | | | |
| | CCGACTACAGCCTGACCATCAGCAACCTGGAGCAGGAGGACATCG | | | |
| | CCACCTACTTCTGCCAGCAGGGCAACACCCTGCCCTACACCTTCGG | | | |
| | CGGCGGCACCAAGCTGGAGCTGAAGAGGGGCGGTGGAGGTTCCG | | | |
| | GCGGTGGCGGTTCCGGAGGCGGTGGGTCAGGAGGTGGAGGCTC | | | |
| | CGAGGTGCAGCTGCAGCAGAGCGGCCCCGGCCTGGTGGCCCCCA | | | |
| | GCCAGAGCCTGAGCGTGACCTGCACCGTGAGCGGCGTGAGCCTG | | | |
| | CCCGACTACGGCGTGAGCTGGATCAGGCAGCCCCCCAGGAAGGG | | | |
| | CCTGGAGTGGCTGGGCGTGATCTGGGGCAGCGAGACCACCTACT | | | |
| | ACAACAGCGCCCTGAAGAGCAGGCTGACCATCATCAAGGACAACA | | | |
| | GCAAGAGCCAGGTGTTCCTGAAGATGAACAGCCTGCAGACCGACG | | | |
| | ACACCGCCATCTACTACTGCGCCAAGCACTACTACTACGGCGGCA | | | |
| | GCTACGCCATGGACTACTGGGGCCAGGGCACCACCGTGACCGTGA | | | |
| | GCAGCTACGTGACCGTGAGCAGCCAGGACCCCGCCGAGCCCAAGA | | | |
| | GCCCCGACAAGACCCACACCTGCCCCCCCTGCCCCGGTGGCGGTG | | | |
| | GAAGTGTGCCCCCCACCCTGGAGGTGACCCAGCAGCCCGTGAGAG | | | |
| | CCGAGAACCAGGTGAACGTGACCTGCCAGGTGAGAAAGTTCTACC | | | |
| | CCCAGAGACTGCAGCTGACCTGGCTGGAGAACGGCAACGTGAGCA | | | |
| | GAACCGAGACCGCCAGCACCGTGACCGAGAACAAGGACGGCACCT | | | |
| | ACAACTGGATGAGCTGGCTGCTGGTGAACGTGAGCGCCCACAGAG | | | |
| | ACGACGTGAAGCTGACCTGCCAGGTGGAGCACGACGGCCAGCCC | | | |
| | GCCGTGAGCAAGAGCCACGACCTGAAGGTGAGCGGCGGAGGCGG | | | |
| | GAGTGCCAAGCCCAGCGCCCCCGTGGTGAGCGGCCCCGCCGCCA | | | |
| | GGGCCACCCCCCAGCACACCGTGAGCTTCACCTGCGAGAGCCACG | | | |
| | GCTTCAGCCCCAGGGACATCACCCTGAAGTGGTTCAAGAACGGCA | | | |
| | ACGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGAG | | | |
| | ACCATCAGGGTGCCCCCCACCCTGGAGGTGACCCAGCAGCCCGTG | | | |
| | AGGGCCGAGAACCAGGTGAACGTGACCTGCCAGGTGAGGAAGTT | | | |
| | CTACCCCCAGAGGCTGCAGCTGACCTGGCTGGAGAACGGCAACGT | | | |
| | GAGCAGGACCGAGACCGCCAGCACCGTGACCGAGAACAAGGACG | | | |
| | GCACCTACAACTGGATGAGCTGGCTGCTGGTGAACGTGAGCGCCC | | | |
| | ACAGGGACGACGTGAAGCTGACCTGCCAGGTGGAGCACGACGGC | | | |
| | CAGCCCGCCGTGAGCAAGAGCCACGACCTGAAGGTGAGCAAGGG | | | |
| | CAAGCACCTGTGCCCCAGCCCCCTGTTCCCCGGCCCCAGCAAGCC | | | |
| | CTTCTGGGTGCTGGTGGTGGTGGGCGGCGTGCTGGCCTGCTACA | | | |
| | GCCTGCTGGTGACCGTGGCCTTCATCATCTTCTGGGTGAGGAGCA | | | |
| | AGAGGAGCAGGCTGCTGCACAGCGACTACATGAACATGACCCCCA | | | |
| | GGAGGCCCGGCCCCACCAGGAAGCACTACCAGCCCTACGCCCCCC | | | |
| | CCAGGGACTTCGCCGCCTACAGGAGCAGGGTGAAGTTCAGCAGGA | | | |
| | GCGCCGACGCCCCCGCCTACCAGCAGGGCCAGAACCAGCTGTACA | | | |
| | ACGAGCTGAACCTGGGCAGGAGGGAGGAGTACGACGTGCTGGAC | | | |
| | AAGAGGAGGGGCAGGGACCCCGAGATGGGCGGCAAGCCCAGGAG | | | |
| | GAAGAACCCCCAGGAGGGCCTGTACAACGAGCTGCAGAAGGACAA | | | |
| | GATGGCCGAGGCCTACAGCGAGATCGGCATGAAGGGCGAGAGGA | | | |
| | GGAGGGGCAAGGGCCACGACGGCCTGTACCAGGGCCTGAGCACC | | | |
| | GCCACCAAGGACACCTACGACGCCCTGCACATGCAGGCCCTGCCC | | | |
| | CCCAGGTAA | | | |
| HER-2 scFv | DIQMTQSPSSLSASVGDRVTITCRASQDVNTAVAWYQQKPGKAPKLLIY | a | a | 53. |
| | SASFLYSGVPSRFSGSRSGTDFTLTISSLQPEDFATYYCQQHYTTPPTF | | | |
| | GQGTKVEIKRGGGGSGGGGSGGGGSGGGGSEVQLVESGGGLVQPG | | | |
| | GSLRLSCAASGFNIKDTYIHWVRQAPGKGLEWVARIYPTNGYTRYADS | | | |
| | VKGRFTISADTSKNTAYLQMNSLRAEDTAVYYCSRWGGDGFYAMDYW | | | |
| | GQGTLVTVS | | | |
| HER-2 CAR M | MEFGLSWLFLVAILKGVQCSRDIQMTQSPSSLSASVGDRVTITCRASQD | a | a | 54. |
| | VNTAVAWYQQKPGKAPKLLIYSASFLYSGVPSRFSGSRSGTDFTLTISSL | | | |
| | QPEDFATYYCQQHYTTPPTFGQGTKVEIKRGGGGSGGGGSGGGGSG | | | |
| | GGGSEVQLVESGGGLVQPGGSLRLSCAASGFNIKDTYIHWVRQAPGK | | | |
| | GLEWVARIYPTNGYTRYADSVKGRFTISADTSKNTAYLQMNSLRAEDT | | | |
| | AVYYCSRWGGDGFYAMDYWGQGTLVTVSSYVTVSSQDPAEPKSPDKT | | | |
| | HTCPPCPGGGGSAKPSAPVVSGPAARATPQHTVSFTCESHGFSPRDIT | | | |
| | LKWFKNGNELSDFQTNVDPVGESVSYSIHSTAKVVLTREDVHSQVICEV | | | |
| | AHVTLQGDPLRGTANLSETIRVPPTLEVTQQPVRAENQVNVTCQVRKF | | | |
| | YPQRLQLTWLENGNVSRTETASTVTENKDGTYNWMSWLLVNVSAHR | | | |
| | DDVKLTCQVEHDGQPAVSKSHDLKVSGGGGSFWVLVVVGGVLACYSL | | | |
| | LVTVAFIIFWVRSKRSRLLHSDYMNMTPRRPGPTRKHYQPYAPPRDFA | | | |
| | AYRSRVKFSRSADAPAYQQGQNQLYNELNLGRREEYDVLDKRRGRDPE | | | |
| | MGGKPRRKNPQEGLYNELQKDKMAEAYSEIGMKGERRRGKGHDGLY | | | |
| | QGLSTATKDTYDALHMQALPPR | | | |
| HER-2 CAR M | ATGGAGTTCGGCCTGAGCTGGCTGTTCCTGGTGGCCATCCTGAAG | n | a | 55. |
| | GGCGTGCAGTGCAGCAGGGACATCCAGATGACCCAGAGCCCCAGC | | | |
| | AGCCTGAGCGCCAGCGTGGGCGACAGGGTGACCATCACCTGCAG | | | |
| | GGCCAGCCAGGACGTGAACACCGCCGTGGCCTGGTACCAGCAGAA | | | |
| | GCCCGGCAAGGCCCCCAAGCTGCTGATCTACAGCGCCAGCTTCCT | | | |
| | GTACAGCGGCGTGCCCAGCAGGTTCAGCGGCAGCAGGAGCGGCA | | | |
| | CCGACTTCACCCTGACCATCAGCAGCCTGCAGCCCGAGGACTTCG | | | |
| | CCACCTACTACTGCCAGCAGCACTACACCACCCCCCCCACCTTCGG | | | |
| | CCAGGGCACCAAGGTGGAGATCAAGAGGGGCGGTGGAGGTTCCG | | | |
| | GCGGTGGCGGTTCCGGAGGCGGTGGGTCAGGAGGTGGAGGCTC | | | |
| | CGAGGTGCAGCTGGTGGAGAGCGGCGGCGGCCTGGTGCAGCCCG | | | |
| | GCGGCAGCCTGAGGCTGAGCTGCGCCGCCAGCGGCTTCAACATCA | | | |
| | AGGACACCTACATCCACTGGGTGAGGCAGGCCCCCGGCAAGGGCC | | | |
| | TGGAGTGGGTGGCCAGGATCTACCCCACCAACGGCTACACCAGGT | | | |
| | ACGCCGACAGCGTGAAGGGCAGGTTCACCATCAGCGCCGACACCA | | | |
| | GCAAGAACACCGCCTACCTGCAGATGAACAGCCTGAGGGCCGAGG | | | |
| | ACACCGCCGTGTACTACTGCAGCAGGTGGGGCGGCGACGGCTTCT | | | |
| | ACGCCATGGACTACTGGGGCCAGGGCACCCTGGTGACCGTGAGC | | | |
| | AGCTACGTGACCGTGAGCAGCCAGGACCCCGCCGAGCCCAAGAGC | | | |
| | CCCGACAAGACCCACACCTGCCCCCCCTGCCCCGGTGGCGGTGGA | | | |
| | AGTGCCAAGCCCAGCGCCCCCGTGGTGAGCGGCCCCGCCGCCAG | | | |
| | GGCCACCCCCCAGCACACCGTGAGCTTCACCTGCGAGAGCCACGG | | | |
| | CTTCAGCCCCAGGGACATCACCCTGAAGTGGTTCAAGAACGGCAA | | | |
| | CGAGCTGAGCGACTTCCAGACCAACGTGGACCCCGTGGGCGAGA | | | |
| | GCGTGAGCTACAGCATCCACAGCACCGCCAAGGTGGTGCTGACCA | | | |
| | GGGAGGACGTGCACAGCCAGGTGATCTGCGAGGTGGCCCACGTG | | | |
| | ACCCTGCAGGGCGACCCCCTGAGGGGCACCGCCAACCTGAGCGAG | | | |
| | ACCATCAGGGTGCCCCCCACCCTGGAGGTGACCCAGCAGCCCGTG | | | |
| | AGGGCCGAGAACCAGGTGAACGTGACCTGCCAGGTGAGGAAGTT | | | |
| | CTACCCCCAGAGGCTGCAGCTGACCTGGCTGGAGAACGGCAACGT | | | |
| | GAGCAGGACCGAGACCGCCAGCACCGTGACCGAGAACAAGGACG | | | |
| | GCACCTACAACTGGATGAGCTGGCTGCTGGTGAACGTGAGCGCCC | | | |
| | ACAGGGACGACGTGAAGCTGACCTGCCAGGTGGAGCACGACGGC | | | |
| | CAGCCCGCCGTGAGCAAGAGCCACGACCTGAAGGTGAGCGGCGG | | | |
| | TGGCGGCAGCTTCTGGGTGCTGGTGGTGGTGGGCGGCGTGCTG | | | |
| | GCCTGCTACAGCCTGCTGGTGACCGTGGCCTTCATCATCTTCTGG | | | |
| | GTGAGGAGCAAGAGGAGCAGGCTGCTGCACAGCGACTACATGAAC | | | |
| | ATGACCCCCAGGAGGCCCGGCCCCACCAGGAAGCACTACCAGCCC | | | |
| | TACGCCCCCCCCAGGGACTTCGCCGCCTACAGGAGCAGGGTGAAG | | | |
| | TTCAGCAGGAGCGCCGACGCCCCCGCCTACCAGCAGGGCCAGAAC | | | |
| | CAGCTGTACAACGAGCTGAACCTGGGCAGGAGGGAGGAGTACGA | | | |
| | CGTGCTGGACAAGAGGAGGGGCAGGGACCCCGAGATGGGCGGCA | | | |
| | AGCCCAGGAGGAAGAACCCCCAGGAGGGCCTGTACAACGAGCTGC | | | |
| | AGAAGGACAAGATGGCCGAGGCCTACAGCGAGATCGGCATGAAG | | | |
| | GGCGAGAGGAGGAGGGGCAAGGGCCACGACGGCCTGTACCAGGG | | | |
| | CCTGAGCACCGCCACCAAGGACACCTACGACGCCCTGCACATGCA | | | |
| | GGCCCTGCCCCCCAGGTAA | | | |
| CAR spacer | ESKYGPPCPPCPGGGGSAKPSAPVVSGPAARATPQHTVSFTCESHGFS | a | a | 56. |
| M1 | PRDITLKWFKNGNELSDFQTNVDPVGESVSYSIHSTAKVVLTREDVHSQ | | | |
| | VICEVAHVTLQGDPLRGTANLSETIRVPPTLEVTQQPVRAENQVNVTC | | | |
| | QVRKFYPQRLQLTWLENGNVSRTETASTVTENKDGTYNWMSWLLVN | | | |
| | VSAHRDDVKLTCQVEHDGQPAVSKSHDLK | | | |
| CAR spacer | ESKYGPPCPPCPGGGGSAKPSAPVVSGPAARATPQHTVSFTCESHGFS | a | a | 57. |
| XM2 | PRDITLKWFKNGNELSDFQTNVDPVGESVSYSIHSTAKVVLTREDVHSQ | | | |
| | VICEVAHVTLQGDPLRGTANLSETIRVPPTLEVTQQPVRAENQVNVTC | | | |
| | QVRKFYPQRLQLTWLENGNVSRTETASTVTENKDGTYNWMSWLLVN | | | |
| | VSAHRDDVKLTCQVEHDGQPAVSKSHDLKVSKGKHLCPSPLFPGPSKP | | | |
| CAR spacer | ESKYGPPCPPCPGGGGSAKPSAPVVSGPAARATPQHTVSFTCESHGFS | a | a | 58. |
| XM3 | PRDITLKWFKNGNELSDFQTNVDPVGESVSYSIHSTAKVVLTREDVHSQ | | | |
| | VICEVAHVTLQGDPLRGTANLSETIRESKYGPPCPPCPGGGGSVPPTLE | | | |
| | VTQQPVRAENQVNVTCQVRKFYPQRLQLTWLENGNVSRTETASTVTE | | | |
| | NKDGTYNWMSWLLVNVSAHRDDVKLTCQVEHDGQPAVSKSHDLKVS | | | |
| | KGKHLCPSPLFPGPSKP | | | |
| CAR spacer M4 | ESKYGPPCPPCPGGGGSAKPSAPVVSGPAARATPQHTVSFTCESHGFS | a | a | 59. |
| | PRDITLKWFKNGNELSDFQTNVDPVGESVSYSIHSTAKVVLTREDVHSQ | | | |
| | VICEVAHVTLQGDPLRGTANLSETIRESKYGPPCPPCPGGGGSVPPTLE | | | |
| | VTQQPVRAENQVNVTCQVRKFYPQRLQLTWLENGNVSRTETASTVTE | | | |
| | NKDGTYNWMSWLLVNVSAHRDDVKLTCQVEHDGQPAVSKSHDLKVS | | | |
| | ESKYGPPCPPCPGGGGS | | | |
| CAR spacer | ESKYGPPCPPCPGGGGSVPPTLEVTQQPVRAENQVNVTCQVRKFYPQ | a | a | 60. |
| 2S5 | RLQLTWLENGNVSRTETASTVTENKDGTYNWMSWLLVNVSAHRDDV | | | |
| | KLTCQVEHDGQPAVSKSHDLKVSESKYGPPCPPCPGGGGS | | | |
| CAR spacer M6 | PSAPVVSGPAARATPQHTVSFTCESHGFSPRDITLKWFKNGNELSDFQ | a | a | 61. |
| | TNVDPVGESVSYSIHSTAKVVLTREDVHSQVICEVAHVTLQGDPLRGTA | | | |
| | NLSETIRVPPTLEVTQQPVRAENQVNVTCQVRKFYPQRLQLTWLENG | | | |
| | NVSRTETASTVTENKDGTYNWMSWLLVNVSAHRDDVKLTCQVEHDG | | | |
| | QPAVSKSHDLKVSGGGGS | | | |
| CAR M1 | MEFGLSWLFLVAILKGVQCSRDIQMTQTTSSLSASLGDRVTISCRASQDI | a | a | 62. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSESKYGPPCPPCPGGGGSAKP | | | |
| | SAPVVSGPAARATPQHTVSFTCESHGFSPRDITLKWFKNGNELSDFQT | | | |
| | NVDPVGESVSYSIHSTAKVVLTREDVHSQVICEVAHVTLQGDPLRGTAN | | | |
| | LSETIRVPPTLEVTQQPVRAENQVNVTCQVRKFYPQRLQLTWLENGN | | | |
| | VSRTETASTVTENKDGTYNWMSWLLVNVSAHRDDVKLTCQVEHDGQ | | | |
| | PAVSKSHDLKVSGGGGSFWVLVVVGGVLACYSLLVTVAFIIFWVRSKRS | | | |
| | RLLHSDYMNMTPRRPGPTRKHYQPYAPPRDFAAYRSRVKFSRSADAPA | | | |
| | YQQGQNQLYNELNLGRREEYDVLDKRRGRDPEMGGKPRRKNPQEGL | | | |
| | YNELQKDKMAEAYSEIGMKGERRRGKGHDGLYQGLSTATKDTYDALH | | | |
| | MQALPPR | | | |
| CAR XM2 | MEFGLSWLFLVAILKGVQCSRDIQMTQTTSSLSASLGDRVTISCRASQDI | a | a | 63. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSESKYGPPCPPCPGGGGSAKP | | | |
| | SAPVVSGPAARATPQHTVSFTCESHGFSPRDITLKWFKNGNELSDFQT | | | |
| | NVDPVGESVSYSIHSTAKVVLTREDVHSQVICEVAHVTLQGDPLRGTAN | | | |
| | LSETIRVPPTLEVTQQPVRAENQVNVTCQVRKFYPQRLQLTWLENGN | | | |
| | VSRTETASTVTENKDGTYNWMSWLLVNVSAHRDDVKLTCQVEHDGQ | | | |
| | PAVSKSHDLKVSKGKHLCPSPLFPGPSKPFWVLVVVGGVLACYSLLVT | | | |
| | VAFIIFWVRSKRSRLLHSDYMNMTPRRPGPTRKHYQPYAPPRDFAAYR | | | |
| | SRVKFSRSADAPAYQQGQNQLYNELNLGRREEYDVLDKRRGRDPEMG | | | |
| | GKPRRKNPQEGLYNELQKDKMAEAYSEIGMKGERRRGKGHDGLYQGL | | | |
| | STATKDTYDALHMQALPPR | | | |
| CAR XM3 | MEFGLSWLFLVAILKGVQCSRDIQMTQTTSSLSASLGDRVTISCRASQDI | a | a | 64. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSESKYGPPCPPCPGGGGSAKP | | | |
| | SAPVVSGPAARATPQHTVSFTCESHGFSPRDITLKWFKNGNELSDFQT | | | |
| | NVDPVGESVSYSIHSTAKVVLTREDVHSQVICEVAHVTLQGDPLRGTAN | | | |
| | LSETIRESKYGPPCPPCPGGGGSVPPTLEVTQQPVRAENQVNVTCQVR | | | |
| | KFYPQRLQLTWLENGNVSRTETASTVTENKDGTYNWMSWLLVNVSA | | | |
| | HRDDVKLTCQVEHDGQPAVSKSHDLKVSKGKHLCPSPLFPGPSKPFW | | | |
| | VLVVVGGVLACYSLLVTVAFIIFWVRSKRSRLLHSDYMNMTPRRPGPTR | | | |
| | KHYQPYAPPRDFAAYRSRVKFSRSADAPAYQQGQNQLYNELNLGRRE | | | |
| | EYDVLDKRRGRDPEMGGKPRRKNPQEGLYNELQKDKMAEAYSEIGMK | | | |
| | GERRRGKGHDGLYQGLSTATKDTYDALHMQALPPR | | | |
| CAR M4 | MEFGLSWLFLVAILKGVQCSRDIQMTQTTSSLSASLGDRVTISCRASQDI | a | a | 65. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSESKYGPPCPPCPGGGGSAKP | | | |
| | SAPVVSGPAARATPQHTVSFTCESHGFSPRDITLKWFKNGNELSDFQT | | | |
| | NVDPVGESVSYSIHSTAKVVLTREDVHSQVICEVAHVTLQGDPLRGTAN | | | |
| | LSETIRESKYGPPCPPCPGGGGSVPPTLEVTQQPVRAENQVNVTCQVR | | | |
| | KFYPQRLQLTWLENGNVSRTETASTVTENKDGTYNWMSWLLVNVSA | | | |
| | HRDDVKLTCQVEHDGQPAVSKSHDLKVSESKYGPPCPPCPGGGGSFW | | | |
| | VLVVVGGVLACYSLLVTVAFIIFWVRSKRSRLLHSDYMNMTPRRPGPTR | | | |
| | KHYQPYAPPRDFAAYRSRVKFSRSADAPAYQQGQNQLYNELNLGRRE | | | |
| | EYDVLDKRRGRDPEMGGKPRRKNPQEGLYNELQKDKMAEAYSEIGMK | | | |
| | GERRRGKGHDGLYQGLSTATKDTYDALHMQALPPR | | | |
| CAR 2S5 | MEFGLSWLFLVAILKGVQCSRDIQMTQTTSSLSASLGDRVTISCRASQDI | a | a | 66. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSESKYGPPCPPCPGGGGSVPP | | | |
| | TLEVTQQPVRAENQVNVTCQVRKFYPQRLQLTWLENGNVSRTETAST | | | |
| | VTENKDGTYNWMSWLLVNVSAHRDDVKLTCQVEHDGQPAVSKSHDL | | | |
| | KVSESKYGPPCPPCPGGGGSFWVLVVVGGVLACYSLLVTVAFIIFWVR | | | |
| | SKRSRLLHSDYMNMTPRRPGPTRKHYQPYAPPRDFAAYRSRVKFSRSA | | | |
| | DAPAYQQGQNQLYNELNLGRREEYDVLDKRRGRDPEMGGKPRRKNP | | | |
| | QEGLYNELQKDKMAEAYSEIGMKGERRRGKGHDGLYQGLSTATKDTY | | | |
| | DALHMQALPPR | | | |
| CAR M6 | MEFGLSWLFLVAILKGVQCSRDIQMTQTTSSLSASLGDRVTISCRASQDI | a | a | 67. |
| | SKYLNWYQQKPDGTVKLLIYHTSRLHSGVPSRFSGSGSGTDYSLTISNL | | | |
| | EQEDIATYFCQQGNTLPYTFGGGTKLELKRGGGGSGGGGSGGGGSG | | | |
| | GGGSEVQLQQSGPGLVAPSQSLSVTCTVSGVSLPDYGVSWIRQPPRKG | | | |
| | LEWLGVIWGSETTYYNSALKSRLTIIKDNSKSQVFLKMNSLQTDDTAIYY | | | |
| | CAKHYYYGGSYAMDYWGQGTTVTVSSGGGGSAKPSAPVVSGPAARA | | | |
| | TPQHTVSFTCESHGFSPRDITLKWFKNGNELSDFQTNVDPVGESVSYSI | | | |
| | HSTAKVVLTREDVHSQVICEVAHVTLQGDPLRGTANLSETIRVPPTLEV | | | |
| | TQQPVRAENQVNVTCQVRKFYPQRLQLTWLENGNVSRTETASTVTEN | | | |
| | KDGTYNWMSWLLVNVSAHRDDVKLTCQVEHDGQPAVSKSHDLKVSG | | | |
| | GGGSFWVLVVVGGVLACYSLLVTVAFIIFWVRSKRSRLLHSDYMNMTP | | | |
| | RRPGPTRKHYQPYAPPRDFAAYRSRVKFSRSADAPAYQQGQNQLYNE | | | |
| | LNLGRREEYDVLDKRRGRDPEMGGKPRRKNPQEGLYNELQKDKMAEA | | | |
| | YSEIGMKGERRRGKGHDGLYQGLSTATKDTYDALHMQALPPR | | | |
| CAR spacer M1 | GAGAGCAAGTACGGCCCCCCCTGCCCCCCCTGCCCCGGTGGCGGT | n | a | 68. |
| | GGAAGTGCCAAGCCCAGCGCCCCCGTGGTGAGCGGCCCCGCCGC | | | |
| | CAGGGCCACCCCCCAGCACACCGTGAGCTTCACCTGCGAGAGCCA | | | |
| | CGGCTTCAGCCCCAGGGACATCACCCTGAAGTGGTTCAAGAACGG | | | |
| | CAACGAGCTGAGCGACTTCCAGACCAACGTGGACCCCGTGGGCGA | | | |
| | GAGCGTGAGCTACAGCATCCACAGCACCGCCAAGGTGGTGCTGAC | | | |
| | CAGGGAGGACGTGCACAGCCAGGTGATCTGCGAGGTGGCCCACG | | | |
| | TGACCCTGCAGGGCGACCCCCTGAGGGGCACCGCCAACCTGAGCG | | | |
| | AGACCATCAGGGTGCCCCCCACCCTGGAGGTGACCCAGCAGCCCG | | | |
| | TGAGGGCCGAGAACCAGGTGAACGTGACCTGCCAGGTGAGGAAG | | | |
| | TTCTACCCCCAGAGGCTGCAGCTGACCTGGCTGGAGAACGGCAAC | | | |
| | GTGAGCAGGACCGAGACCGCCAGCACCGTGACCGAGAACAAGGAC | | | |
| | GGCACCTACAACTGGATGAGCTGGCTGCTGGTGAACGTGAGCGCC | | | |
| | CACAGGGACGACGTGAAGCTGACCTGCCAGGTGGAGCACGACGG | | | |
| | CCAGCCCGCCGTGAGCAAGAGCCACGACCTGAAGGTGAGC | | | |
| CAR spacer | GAGAGCAAGTACGGCCCCCCCTGCCCCCCCTGCCCCGGTGGCGGT | n | a | 69. |
| XM2 | GGAAGTGCCAAGCCCAGCGCCCCCGTGGTGAGCGGCCCCGCCGC | | | |
| | CAGGGCCACCCCCCAGCACACCGTGAGCTTCACCTGCGAGAGCCA | | | |
| | CGGCTTCAGCCCCAGGGACATCACCCTGAAGTGGTTCAAGAACGG | | | |
| | CAACGAGCTGAGCGACTTCCAGACCAACGTGGACCCCGTGGGCGA | | | |
| | GAGCGTGAGCTACAGCATCCACAGCACCGCCAAGGTGGTGCTGAC | | | |
| | CAGGGAGGACGTGCACAGCCAGGTGATCTGCGAGGTGGCCCACG | | | |
| | TGACCCTGCAGGGCGACCCCCTGAGGGGCACCGCCAACCTGAGCG | | | |
| | AGACCATCAGGGTGCCCCCCACCCTGGAGGTGACCCAGCAGCCCG | | | |
| | TGAGGGCCGAGAACCAGGTGAACGTGACCTGCCAGGTGAGGAAG | | | |
| | TTCTACCCCCAGAGGCTGCAGCTGACCTGGCTGGAGAACGGCAAC | | | |
| | GTGAGCAGGACCGAGACCGCCAGCACCGTGACCGAGAACAAGGAC | | | |
| | GGCACCTACAACTGGATGAGCTGGCTGCTGGTGAACGTGAGCGCC | | | |
| | CACAGGGACGACGTGAAGCTGACCTGCCAGGTGGAGCACGACGG | | | |
| | CCAGCCCGCCGTGAGCAAGAGCCACGACCTGAAGGTGAGCAAGG | | | |
| | GCAAGCACCTGTGCCCCAGCCCCCTGTTCCCCGGCCCCAGCAAGC | | | |
| | CC | | | |
| CAR spacer | GAGAGCAAGTACGGCCCCCCCTGCCCCCCCTGCCCCGGTGGCGGT | n | a | 70. |
| XM3 | GGAAGTGCCAAGCCCAGCGCCCCCGTGGTGAGCGGCCCCGCCGC | | | |
| | CAGGGCCACCCCCCAGCACACCGTGAGCTTCACCTGCGAGAGCCA | | | |
| | CGGCTTCAGCCCCAGGGACATCACCCTGAAGTGGTTCAAGAACGG | | | |
| | CAACGAGCTGAGCGACTTCCAGACCAACGTGGACCCCGTGGGCGA | | | |
| | GAGCGTGAGCTACAGCATCCACAGCACCGCCAAGGTGGTGCTGAC | | | |
| | CAGGGAGGACGTGCACAGCCAGGTGATCTGCGAGGTGGCCCACG | | | |
| | TGACCCTGCAGGGCGACCCCCTGAGGGGCACCGCCAACCTGAGCG | | | |
| | AGACCATCAGGGAATCCAAATACGGACCACCATGCCCACCATGCCC | | | |
| | AGGCGGAGGCGGTAGTGTGCCCCCCACCCTGGAGGTGACCCAGC | | | |
| | AGCCCGTGAGGGCCGAGAACCAGGTGAACGTGACCTGCCAGGTG | | | |
| | AGGAAGTTCTACCCCCAGAGGCTGCAGCTGACCTGGCTGGAGAAC | | | |
| | GGCAACGTGAGCAGGACCGAGACCGCCAGCACCGTGACCGAGAAC | | | |
| | AAGGACGGCACCTACAACTGGATGAGCTGGCTGCTGGTGAACGTG | | | |
| | AGCGCCCACAGGGACGACGTGAAGCTGACCTGCCAGGTGGAGCA | | | |
| | CGACGGCCAGCCCGCCGTGAGCAAGAGCCACGACCTGAAGGTGA | | | |
| | GCAAGGGCAAGCACCTGTGCCCCAGCCCCCTGTTCCCCGGCCCCA | | | |
| | GCAAGCCC | | | |
| CAR spacer M4 | GAGAGCAAGTACGGCCCCCCCTGCCCCCCCTGCCCCGGTGGCGGT | n | a | 71. |
| | GGAAGTGCCAAGCCCAGCGCCCCCGTGGTGAGCGGCCCCGCCGC | | | |
| | CAGGGCCACCCCCCAGCACACCGTGAGCTTCACCTGCGAGAGCCA | | | |
| | CGGCTTCAGCCCCAGGGACATCACCCTGAAGTGGTTCAAGAACGG | | | |
| | CAACGAGCTGAGCGACTTCCAGACCAACGTGGACCCCGTGGGCGA | | | |
| | GAGCGTGAGCTACAGCATCCACAGCACCGCCAAGGTGGTGCTGAC | | | |
| | CAGGGAGGACGTGCACAGCCAGGTGATCTGCGAGGTGGCCCACG | | | |
| | TGACCCTGCAGGGCGACCCCCTGAGGGGCACCGCCAACCTGAGCG | | | |
| | AGACCATCAGGGAATCCAAATACGGACCACCATGCCCACCATGCCC | | | |
| | AGGAGGTGGCGGAAGTGTGCCCCCCACCCTGGAGGTGACCCAGC | | | |
| | AGCCCGTGAGGGCCGAGAACCAGGTGAACGTGACCTGCCAGGTG | | | |
| | AGGAAGTTCTACCCCCAGAGGCTGCAGCTGACCTGGCTGGAGAAC | | | |
| | GGCAACGTGAGCAGGACCGAGACCGCCAGCACCGTGACCGAGAAC | | | |
| | AAGGACGGCACCTACAACTGGATGAGCTGGCTGCTGGTGAACGTG | | | |
| | AGCGCCCACAGGGACGACGTGAAGCTGACCTGCCAGGTGGAGCA | | | |
| | CGACGGCCAGCCCGCCGTGAGCAAGAGCCACGACCTGAAGGTGA | | | |
| | GCGAATCCAAATACGGACCACCATGCCCACCATGCCCAGGCGGTG | | | |
| | GCGGCAGC | | | |
| CAR spacer | GAGAGCAAGTACGGCCCCCCCTGCCCCCCCTGCCCCGGTGGCGGT | n | a | 72. |
| 2S5 | GGAAGTGTGCCCCCCACCCTGGAGGTGACCCAGCAGCCCGTGAG | | | |
| | GGCCGAGAACCAGGTGAACGTGACCTGCCAGGTGAGGAAGTTCTA | | | |
| | CCCCCAGAGGCTGCAGCTGACCTGGCTGGAGAACGGCAACGTGA | | | |
| | GCAGGACCGAGACCGCCAGCACCGTGACCGAGAACAAGGACGGCA | | | |
| | CCTACAACTGGATGAGCTGGCTGCTGGTGAACGTGAGCGCCCACA | | | |
| | GGGACGACGTGAAGCTGACCTGCCAGGTGGAGCACGACGGCCAG | | | |
| | CCCGCCGTGAGCAAGAGCCACGACCTGAAGGTGAGCGAATCCAAA | | | |
| | TACGGACCACCATGCCCACCATGCCCAGGCGGTGGCGGCAGC | | | |
| CAR spacer M6 | GGTGGCGGTGGAAGTGCCAAGCCCAGCGCCCCCGTGGTGAGCGG | n | a | 73. |
| | CCCCGCCGCCAGGGCCACCCCCCAGCACACCGTGAGCTTCACCTG | | | |
| | CGAGAGCCACGGCTTCAGCCCCAGGGACATCACCCTGAAGTGGTT | | | |
| | CAAGAACGGCAACGAGCTGAGCGACTTCCAGACCAACGTGGACCC | | | |
| | CGTGGGCGAGAGCGTGAGCTACAGCATCCACAGCACCGCCAAGGT | | | |
| | GGTGCTGACCAGGGAGGACGTGCACAGCCAGGTGATCTGCGAGG | | | |
| | TGGCCCACGTGACCCTGCAGGGCGACCCCCTGAGGGGCACCGCCA | | | |
| | ACCTGAGCGAGACCATCAGGGTGCCCCCCACCCTGGAGGTGACCC | | | |
| | AGCAGCCCGTGAGGGCCGAGAACCAGGTGAACGTGACCTGCCAG | | | |
| | GTGAGGAAGTTCTACCCCCAGAGGCTGCAGCTGACCTGGCTGGAG | | | |
| | AACGGCAACGTGAGCAGGACCGAGACCGCCAGCACCGTGACCGAG | | | |
| | AACAAGGACGGCACCTACAACTGGATGAGCTGGCTGCTGGTGAAC | | | |
| | GTGAGCGCCCACAGGGACGACGTGAAGCTGACCTGCCAGGTGGA | | | |
| | GCACGACGGCCAGCCCGCCGTGAGCAAGAGCCACGACCTGAAGGT | | | |
| | GAGCGGCGGTGGCGGCAGC | | | |
| CAR M1 | GCCACCATGGAGTTCGGCCTGAGCTGGCTGTTCCTGGTGGCCATC | n | a | 74. |
| | CTGAAGGGCGTGCAGTGCAGCAGGGACATCCAGATGACCCAGACC | | | |
| | ACCAGCAGCCTGAGCGCCAGCCTGGGCGACAGGGTGACCATCAGC | | | |
| | TGCAGGGCCAGCCAGGACATCAGCAAGTACCTGAACTGGTACCAG | | | |
| | CAGAAGCCCGACGGCACCGTGAAGCTGCTGATCTACCACACCAGC | | | |
| | AGGCTGCACAGCGGCGTGCCCAGCAGGTTCAGCGGCAGCGGCAG | | | |
| | CGGCACCGACTACAGCCTGACCATCAGCAACCTGGAGCAGGAGGA | | | |
| | CATCGCCACCTACTTCTGCCAGCAGGGCAACACCCTGCCCTACACC | | | |
| | TTCGGCGGCGGCACCAAGCTGGAGCTGAAGAGGGGCGGTGGAGG | | | |
| | TTCCGGCGGTGGCGGTTCCGGAGGCGGTGGGTCAGGAGGTGGA | | | |
| | GGCTCCGAGGTGCAGCTGCAGCAGAGCGGCCCCGGCCTGGTGGC | | | |
| | CCCCAGCCAGAGCCTGAGCGTGACCTGCACCGTGAGCGGCGTGA | | | |
| | GCCTGCCCGACTACGGCGTGAGCTGGATCAGGCAGCCCCCCAGGA | | | |
| | AGGGCCTGGAGTGGCTGGGCGTGATCTGGGGCAGCGAGACCACC | | | |
| | TACTACAACAGCGCCCTGAAGAGCAGGCTGACCATCATCAAGGAC | | | |
| | AACAGCAAGAGCCAGGTGTTCCTGAAGATGAACAGCCTGCAGACC | | | |
| | GACGACACCGCCATCTACTACTGCGCCAAGCACTACTACTACGGC | | | |
| | GGCAGCTACGCCATGGACTACTGGGGCCAGGGCACCACCGTGACC | | | |
| | GTGAGCAGCGAGAGCAAGTACGGCCCCCCCTGCCCCCCCTGCCCC | | | |
| | GGTGGCGGTGGAAGTGCCAAGCCCAGCGCCCCCGTGGTGAGCGG | | | |
| | CCCCGCCGCCAGGGCCACCCCCCAGCACACCGTGAGCTTCACCTG | | | |
| | CGAGAGCCACGGCTTCAGCCCCAGGGACATCACCCTGAAGTGGTT | | | |
| | CAAGAACGGCAACGAGCTGAGCGACTTCCAGACCAACGTGGACCC | | | |
| | CGTGGGCGAGAGCGTGAGCTACAGCATCCACAGCACCGCCAAGGT | | | |
| | GGTGCTGACCAGGGAGGACGTGCACAGCCAGGTGATCTGCGAGG | | | |
| | TGGCCCACGTGACCCTGCAGGGCGACCCCCTGAGGGGCACCGCCA | | | |
| | ACCTGAGCGAGACCATCAGGGTGCCCCCCACCCTGGAGGTGACCC | | | |
| | AGCAGCCCGTGAGGGCCGAGAACCAGGTGAACGTGACCTGCCAG | | | |
| | GTGAGGAAGTTCTACCCCCAGAGGCTGCAGCTGACCTGGCTGGAG | | | |
| | AACGGCAACGTGAGCAGGACCGAGACCGCCAGCACCGTGACCGAG | | | |
| | AACAAGGACGGCACCTACAACTGGATGAGCTGGCTGCTGGTGAAC | | | |
| | GTGAGCGCCCACAGGGACGACGTGAAGCTGACCTGCCAGGTGGA | | | |
| | GCACGACGGCCAGCCCGCCGTGAGCAAGAGCCACGACCTGAAGGT | | | |
| | GAGCGGCGGTGGCGGCAGCTTCTGGGTGCTGGTGGTGGTGGGC | | | |
| | GGCGTGCTGGCCTGCTACAGCCTGCTGGTGACCGTGGCCTTCATC | | | |
| | ATCTTCTGGGTGAGGAGCAAGAGGAGCAGGCTGCTGCACAGCGA | | | |
| | CTACATGAACATGACCCCCAGGAGGCCCGGCCCCACCAGGAAGCA | | | |
| | CTACCAGCCCTACGCCCCCCCCAGGGACTTCGCCGCCTACAGGAG | | | |
| | CAGGGTGAAGTTCAGCAGGAGCGCCGACGCCCCCGCCTACCAGCA | | | |
| | GGGCCAGAACCAGCTGTACAACGAGCTGAACCTGGGCAGGAGGG | | | |
| | AGGAGTACGACGTGCTGGACAAGAGGAGGGGCAGGGACCCCGAG | | | |
| | ATGGGCGGCAAGCCCAGGAGGAAGAACCCCCAGGAGGGCCTGTA | | | |
| | CAACGAGCTGCAGAAGGACAAGATGGCCGAGGCCTACAGCGAGAT | | | |
| | CGGCATGAAGGGCGAGAGGAGGAGGGGCAAGGGCCACGACGGCC | | | |
| | TGTACCAGGGCCTGAGCACCGCCACCAAGGACACCTACGACGCCC | | | |
| | TGCACATGCAGGCCCTGCCCCCCAGGTAA | | | |
| CAR XM2 | GCCACCATGGAGTTCGGCCTGAGCTGGCTGTTCCTGGTGGCCATC | n | a | 75. |
| | CTGAAGGGCGTGCAGTGCAGCAGGGACATCCAGATGACCCAGACC | | | |
| | ACCAGCAGCCTGAGCGCCAGCCTGGGCGACAGGGTGACCATCAGC | | | |
| | TGCAGGGCCAGCCAGGACATCAGCAAGTACCTGAACTGGTACCAG | | | |
| | CAGAAGCCCGACGGCACCGTGAAGCTGCTGATCTACCACACCAGC | | | |
| | AGGCTGCACAGCGGCGTGCCCAGCAGGTTCAGCGGCAGCGGCAG | | | |
| | CGGCACCGACTACAGCCTGACCATCAGCAACCTGGAGCAGGAGGA | | | |
| | CATCGCCACCTACTTCTGCCAGCAGGGCAACACCCTGCCCTACACC | | | |
| | TTCGGCGGCGGCACCAAGCTGGAGCTGAAGAGGGGCGGTGGAGG | | | |
| | TTCCGGCGGTGGCGGTTCCGGAGGCGGTGGGTCAGGAGGTGGA | | | |
| | GGCTCCGAGGTGCAGCTGCAGCAGAGCGGCCCCGGCCTGGTGGC | | | |
| | CCCCAGCCAGAGCCTGAGCGTGACCTGCACCGTGAGCGGCGTGA | | | |
| | GCCTGCCCGACTACGGCGTGAGCTGGATCAGGCAGCCCCCCAGGA | | | |
| | AGGGCCTGGAGTGGCTGGGCGTGATCTGGGGCAGCGAGACCACC | | | |
| | TACTACAACAGCGCCCTGAAGAGCAGGCTGACCATCATCAAGGAC | | | |
| | AACAGCAAGAGCCAGGTGTTCCTGAAGATGAACAGCCTGCAGACC | | | |
| | GACGACACCGCCATCTACTACTGCGCCAAGCACTACTACTACGGC | | | |
| | GGCAGCTACGCCATGGACTACTGGGGCCAGGGCACCACCGTGACC | | | |
| | GTGAGCAGCGAGAGCAAGTACGGCCCCCCCTGCCCCCCCTGCCCC | | | |
| | GGTGGCGGTGGAAGTGCCAAGCCCAGCGCCCCCGTGGTGAGCGG | | | |
| | CCCCGCCGCCAGGGCCACCCCCCAGCACACCGTGAGCTTCACCTG | | | |
| | CGAGAGCCACGGCTTCAGCCCCAGGGACATCACCCTGAAGTGGTT | | | |
| | CAAGAACGGCAACGAGCTGAGCGACTTCCAGACCAACGTGGACCC | | | |
| | CGTGGGCGAGAGCGTGAGCTACAGCATCCACAGCACCGCCAAGGT | | | |
| | GGTGCTGACCAGGGAGGACGTGCACAGCCAGGTGATCTGCGAGG | | | |
| | TGGCCCACGTGACCCTGCAGGGCGACCCCCTGAGGGGCACCGCCA | | | |
| | ACCTGAGCGAGACCATCAGGGTGCCCCCCACCCTGGAGGTGACCC | | | |
| | AGCAGCCCGTGAGGGCCGAGAACCAGGTGAACGTGACCTGCCAG | | | |
| | GTGAGGAAGTTCTACCCCCAGAGGCTGCAGCTGACCTGGCTGGAG | | | |
| | AACGGCAACGTGAGCAGGACCGAGACCGCCAGCACCGTGACCGAG | | | |
| | AACAAGGACGGCACCTACAACTGGATGAGCTGGCTGCTGGTGAAC | | | |
| | GTGAGCGCCCACAGGGACGACGTGAAGCTGACCTGCCAGGTGGA | | | |
| | GCACGACGGCCAGCCCGCCGTGAGCAAGAGCCACGACCTGAAGGT | | | |
| | GAGCAAGGGCAAGCACCTGTGCCCCAGCCCCCTGTTCCCCGGCCC | | | |
| | CAGCAAGCCCTTCTGGGTGCTGGTGGTGGTGGGCGGCGTGCTGG | | | |
| | CCTGCTACAGCCTGCTGGTGACCGTGGCCTTCATCATCTTCTGGG | | | |
| | TGAGGAGCAAGAGGAGCAGGCTGCTGCACAGCGACTACATGAACA | | | |
| | TGACCCCCAGGAGGCCCGGCCCCACCAGGAAGCACTACCAGCCCT | | | |
| | ACGCCCCCCCCAGGGACTTCGCCGCCTACAGGAGCAGGGTGAAGT | | | |
| | TCAGCAGGAGCGCCGACGCCCCCGCCTACCAGCAGGGCCAGAACC | | | |
| | AGCTGTACAACGAGCTGAACCTGGGCAGGAGGGAGGAGTACGAC | | | |
| | GTGCTGGACAAGAGGAGGGGCAGGGACCCCGAGATGGGCGGCAA | | | |
| | GCCCAGGAGGAAGAACCCCCAGGAGGGCCTGTACAACGAGCTGCA | | | |
| | GAAGGACAAGATGGCCGAGGCCTACAGCGAGATCGGCATGAAGG | | | |
| | GCGAGAGGAGGAGGGGCAAGGGCCACGACGGCCTGTACCAGGGC | | | |
| | CTGAGCACCGCCACCAAGGACACCTACGACGCCCTGCACATGCAG | | | |
| | GCCCTGCCCCCCAGGTAA | | | |
| CAR XM3 | GCCACCATGGAGTTCGGCCTGAGCTGGCTGTTCCTGGTGGCCATC | n | a | 76. |
| | CTGAAGGGCGTGCAGTGCAGCAGGGACATCCAGATGACCCAGACC | | | |
| | ACCAGCAGCCTGAGCGCCAGCCTGGGCGACAGGGTGACCATCAGC | | | |
| | TGCAGGGCCAGCCAGGACATCAGCAAGTACCTGAACTGGTACCAG | | | |
| | CAGAAGCCCGACGGCACCGTGAAGCTGCTGATCTACCACACCAGC | | | |
| | AGGCTGCACAGCGGCGTGCCCAGCAGGTTCAGCGGCAGCGGCAG | | | |
| | CGGCACCGACTACAGCCTGACCATCAGCAACCTGGAGCAGGAGGA | | | |
| | CATCGCCACCTACTTCTGCCAGCAGGGCAACACCCTGCCCTACACC | | | |
| | TTCGGGGGGGGCACCAAGCTGGAGCTGAAGAGGGGCGGTGGAGG | | | |
| | TTCCGGCGGTGGCGGTTCCGGAGGCGGTGGGTCAGGAGGTGGA | | | |
| | GGCTCCGAGGTGCAGCTGCAGCAGAGCGGCCCCGGCCTGGTGGC | | | |
| | CCCCAGCCAGAGCCTGAGCGTGACCTGCACCGTGAGCGGCGTGA | | | |
| | GCCTGCCCGACTACGGCGTGAGCTGGATCAGGCAGCCCCCCAGGA | | | |
| | AGGGCCTGGAGTGGCTGGGCGTGATCTGGGGCAGCGAGACCACC | | | |
| | TACTACAACAGCGCCCTGAAGAGCAGGCTGACCATCATCAAGGAC | | | |
| | AACAGCAAGAGCCAGGTGTTCCTGAAGATGAACAGCCTGCAGACC | | | |
| | GACGACACCGCCATCTACTACTGCGCCAAGCACTACTACTACGGC | | a | 77. |
| | GGCAGCTACGCCATGGACTACTGGGGCCAGGGCACCACCGTGACC | | | |
| | GTGAGCAGCGAGAGCAAGTACGGCCCCCCCTGCCCCCCCTGCCCC | | | |
| | GGTGGCGGTGGAAGTGCCAAGCCCAGCGCCCCCGTGGTGAGCGG | | | |
| | CCCCGCCGCCAGGGCCACCCCCCAGCACACCGTGAGCTTCACCTG | | | |
| | CGAGAGCCACGGCTTCAGCCCCAGGGACATCACCCTGAAGTGGTT | | | |
| | CAAGAACGGCAACGAGCTGAGCGACTTCCAGACCAACGTGGACCC | | | |
| | CGTGGGCGAGAGCGTGAGCTACAGCATCCACAGCACCGCCAAGGT | | | |
| | GGTGCTGACCAGGGAGGACGTGCACAGCCAGGTGATCTGCGAGG | | | |
| | TGGCCCACGTGACCCTGCAGGGCGACCCCCTGAGGGGCACCGCCA | | | |
| | ACCTGAGCGAGACCATCAGGGAATCCAAATACGGACCACCATGCC | | | |
| | CACCATGCCCAGGCGGAGGCGGTAGTGTGCCCCCCACCCTGGAG | | | |
| | GTGACCCAGCAGCCCGTGAGGGCCGAGAACCAGGTGAACGTGAC | | | |
| | CTGCCAGGTGAGGAAGTTCTACCCCCAGAGGCTGCAGCTGACCTG | | | |
| | GCTGGAGAACGGCAACGTGAGCAGGACCGAGACCGCCAGCACCG | | | |
| | TGACCGAGAACAAGGACGGCACCTACAACTGGATGAGCTGGCTGC | | | |
| | TGGTGAACGTGAGCGCCCACAGGGACGACGTGAAGCTGACCTGC | | | |
| | CAGGTGGAGCACGACGGCCAGCCCGCCGTGAGCAAGAGCCACGA | | | |
| | CCTGAAGGTGAGCAAGGGCAAGCACCTGTGCCCCAGCCCCCTGTT | | | |
| | CCCCGGCCCCAGCAAGCCCTTCTGGGTGCTGGTGGTGGTGGGCG | | | |
| | GCGTGCTGGCCTGCTACAGCCTGCTGGTGACCGTGGCCTTCATCA | | | |
| | TCTTCTGGGTGAGGAGCAAGAGGAGCAGGCTGCTGCACAGCGAC | | | |
| | TACATGAACATGACCCCCAGGAGGCCCGGCCCCACCAGGAAGCAC | | | |
| | TACCAGCCCTACGCCCCCCCCAGGGACTTCGCCGCCTACAGGAGC | | | |
| | AGGGTGAAGTTCAGCAGGAGCGCCGACGCCCCCGCCTACCAGCAG | | | |
| | GGCCAGAACCAGCTGTACAACGAGCTGAACCTGGGCAGGAGGGA | | | |
| | GGAGTACGACGTGCTGGACAAGAGGAGGGGCAGGGACCCCGAGA | | | |
| | TGGGCGGCAAGCCCAGGAGGAAGAACCCCCAGGAGGGCCTGTAC | | | |
| | AACGAGCTGCAGAAGGACAAGATGGCCGAGGCCTACAGCGAGATC | | | |
| | GGCATGAAGGGCGAGAGGAGGAGGGGCAAGGGCCACGACGGCCT | | | |
| | GTACCAGGGCCTGAGCACCGCCACCAAGGACACCTACGACGCCCT | | | |
| | GCACATGCAGGCCCTGCCCCCCAGGTAA | | | |
| CAR M4 | GCCACCATGGAGTTCGGCCTGAGCTGGCTGTTCCTGGTGGCCATC | n | | |
| | CTGAAGGGCGTGCAGTGCAGCAGGGACATCCAGATGACCCAGACC | | | |
| | ACCAGCAGCCTGAGCGCCAGCCTGGGCGACAGGGTGACCATCAGC | | | |
| | TGCAGGGCCAGCCAGGACATCAGCAAGTACCTGAACTGGTACCAG | | | |
| | CAGAAGCCCGACGGCACCGTGAAGCTGCTGATCTACCACACCAGC | | | |
| | AGGCTGCACAGCGGCGTGCCCAGCAGGTTCAGCGGCAGCGGCAG | | | |
| | CGGCACCGACTACAGCCTGACCATCAGCAACCTGGAGCAGGAGGA | | | |
| | CATCGCCACCTACTTCTGCCAGCAGGGCAACACCCTGCCCTACACC | | | |
| | TTCGGCGGCGGCACCAAGCTGGAGCTGAAGAGGGGCGGTGGAGG | | | |
| | TTCCGGCGGTGGCGGTTCCGGAGGCGGTGGGTCAGGAGGTGGA | | | |
| | GGCTCCGAGGTGCAGCTGCAGCAGAGCGGCCCCGGCCTGGTGGC | | | |
| | CCCCAGCCAGAGCCTGAGCGTGACCTGCACCGTGAGCGGCGTGA | | | |
| | GCCTGCCCGACTACGGCGTGAGCTGGATCAGGCAGCCCCCCAGGA | | | |
| | AGGGCCTGGAGTGGCTGGGCGTGATCTGGGGCAGCGAGACCACC | | | |
| | TACTACAACAGCGCCCTGAAGAGCAGGCTGACCATCATCAAGGAC | | | |
| | AACAGCAAGAGCCAGGTGTTCCTGAAGATGAACAGCCTGCAGACC | | | |
| | GACGACACCGCCATCTACTACTGCGCCAAGCACTACTACTACGGC | | | |
| | GGCAGCTACGCCATGGACTACTGGGGCCAGGGCACCACCGTGACC | | | |
| | GTGAGCAGCGAGAGCAAGTACGGCCCCCCCTGCCCCCCCTGCCCC | | | |
| | GGTGGCGGTGGAAGTGCCAAGCCCAGCGCCCCCGTGGTGAGCGG | | | |
| | CCCCGCCGCCAGGGCCACCCCCCAGCACACCGTGAGCTTCACCTG | | | |
| | CGAGAGCCACGGCTTCAGCCCCAGGGACATCACCCTGAAGTGGTT | | | |
| | CAAGAACGGCAACGAGCTGAGCGACTTCCAGACCAACGTGGACCC | | | |
| | CGTGGGCGAGAGCGTGAGCTACAGCATCCACAGCACCGCCAAGGT | | | |
| | GGTGCTGACCAGGGAGGACGTGCACAGCCAGGTGATCTGCGAGG | | | |
| | TGGCCCACGTGACCCTGCAGGGCGACCCCCTGAGGGGCACCGCCA | | | |
| | ACCTGAGCGAGACCATCAGGGAATCCAAATACGGACCACCATGCC | | | |
| | CACCATGCCCAGGAGGTGGCGGAAGTGTGCCCCCCACCCTGGAG | | | |
| | GTGACCCAGCAGCCCGTGAGGGCCGAGAACCAGGTGAACGTGAC | | | |
| | CTGCCAGGTGAGGAAGTTCTACCCCCAGAGGCTGCAGCTGACCTG | | | |
| | GCTGGAGAACGGCAACGTGAGCAGGACCGAGACCGCCAGCACCG | | | |
| | TGACCGAGAACAAGGACGGCACCTACAACTGGATGAGCTGGCTGC | | | |
| | TGGTGAACGTGAGCGCCCACAGGGACGACGTGAAGCTGACCTGC | | | |
| | CAGGTGGAGCACGACGGCCAGCCCGCCGTGAGCAAGAGCCACGA | | | |
| | CCTGAAGGTGAGCGAATCCAAATACGGACCACCATGCCCACCATG | | | |
| | CCCAGGCGGTGGCGGCAGCTTCTGGGTGCTGGTGGTGGTGGGCG | | | |
| | GCGTGCTGGCCTGCTACAGCCTGCTGGTGACCGTGGCCTTCATCA | | | |
| | TCTTCTGGGTGAGGAGCAAGAGGAGCAGGCTGCTGCACAGCGAC | | | |
| | TACATGAACATGACCCCCAGGAGGCCCGGCCCCACCAGGAAGCAC | | | |
| | TACCAGCCCTACGCCCCCCCCAGGGACTTCGCCGCCTACAGGAGC | | | |
| | AGGGTGAAGTTCAGCAGGAGCGCCGACGCCCCCGCCTACCAGCAG | | | |
| | GGCCAGAACCAGCTGTACAACGAGCTGAACCTGGGCAGGAGGGA | | | |
| | GGAGTACGACGTGCTGGACAAGAGGAGGGGCAGGGACCCCGAGA | | | |
| | TGGGCGGCAAGCCCAGGAGGAAGAACCCCCAGGAGGGCCTGTAC | | | |
| | AACGAGCTGCAGAAGGACAAGATGGCCGAGGCCTACAGCGAGATC | | | |
| | GGCATGAAGGGCGAGAGGAGGAGGGGCAAGGGCCACGACGGCCT | | | |
| | GTACCAGGGCCTGAGCACCGCCACCAAGGACACCTACGACGCCCT | | | |
| | GCACATGCAGGCCCTGCCCCCCAGGTAA | | | |
| CAR 2S5 | GCCACCATGGAGTTCGGCCTGAGCTGGCTGTTCCTGGTGGCCATC | n | a | 78. |
| | CTGAAGGGCGTGCAGTGCAGCAGGGACATCCAGATGACCCAGACC | | | |
| | ACCAGCAGCCTGAGCGCCAGCCTGGGCGACAGGGTGACCATCAGC | | | |
| | TGCAGGGCCAGCCAGGACATCAGCAAGTACCTGAACTGGTACCAG | | | |
| | CAGAAGCCCGACGGCACCGTGAAGCTGCTGATCTACCACACCAGC | | | |
| | AGGCTGCACAGCGGCGTGCCCAGCAGGTTCAGCGGCAGCGGCAG | | | |
| | CGGCACCGACTACAGCCTGACCATCAGCAACCTGGAGCAGGAGGA | | | |
| | CATCGCCACCTACTTCTGCCAGCAGGGCAACACCCTGCCCTACACC | | | |
| | TTCGGCGGCGGCACCAAGCTGGAGCTGAAGAGGGGCGGTGGAGG | | | |
| | TTCCGGCGGTGGCGGTTCCGGAGGCGGTGGGTCAGGAGGTGGA | | | |
| | GGCTCCGAGGTGCAGCTGCAGCAGAGCGGCCCCGGCCTGGTGGC | | | |
| | CCCCAGCCAGAGCCTGAGCGTGACCTGCACCGTGAGCGGCGTGA | | | |
| | GCCTGCCCGACTACGGCGTGAGCTGGATCAGGCAGCCCCCCAGGA | | | |
| | AGGGCCTGGAGTGGCTGGGCGTGATCTGGGGCAGCGAGACCACC | | | |
| | TACTACAACAGCGCCCTGAAGAGCAGGCTGACCATCATCAAGGAC | | | |
| | AACAGCAAGAGCCAGGTGTTCCTGAAGATGAACAGCCTGCAGACC | | | |
| | GACGACACCGCCATCTACTACTGCGCCAAGCACTACTACTACGGC | | | |
| | GGCAGCTACGCCATGGACTACTGGGGCCAGGGCACCACCGTGACC | | | |
| | GTGAGCAGCGAGAGCAAGTACGGCCCCCCCTGCCCCCCCTGCCCC | | | |
| | GGTGGCGGTGGAAGTGTGCCCCCCACCCTGGAGGTGACCCAGCA | | | |
| | GCCCGTGAGGGCCGAGAACCAGGTGAACGTGACCTGCCAGGTGA | | | |
| | GGAAGTTCTACCCCCAGAGGCTGCAGCTGACCTGGCTGGAGAACG | | | |
| | GCAACGTGAGCAGGACCGAGACCGCCAGCACCGTGACCGAGAACA | | | |
| | AGGACGGCACCTACAACTGGATGAGCTGGCTGCTGGTGAACGTGA | | | |
| | GCGCCCACAGGGACGACGTGAAGCTGACCTGCCAGGTGGAGCAC | | | |
| | GACGGCCAGCCCGCCGTGAGCAAGAGCCACGACCTGAAGGTGAG | | | |
| | CGAATCCAAATACGGACCACCATGCCCACCATGCCCAGGCGGTGG | | | |
| | CGGCAGCTTCTGGGTGCTGGTGGTGGTGGGCGGCGTGCTGGCCT | | | |
| | GCTACAGCCTGCTGGTGACCGTGGCCTTCATCATCTTCTGGGTGA | | | |
| | GGAGCAAGAGGAGCAGGCTGCTGCACAGCGACTACATGAACATGA | | | |
| | CCCCCAGGAGGCCCGGCCCCACCAGGAAGCACTACCAGCCCTACG | | | |
| | CCCCCCCCAGGGACTTCGCCGCCTACAGGAGCAGGGTGAAGTTCA | | | |
| | GCAGGAGCGCCGACGCCCCCGCCTACCAGCAGGGCCAGAACCAGC | | | |
| | TGTACAACGAGCTGAACCTGGGCAGGAGGGAGGAGTACGACGTG | | | |
| | CTGGACAAGAGGAGGGGCAGGGACCCCGAGATGGGCGGCAAGCC | | | |
| | CAGGAGGAAGAACCCCCAGGAGGGCCTGTACAACGAGCTGCAGAA | | | |
| | GGACAAGATGGCCGAGGCCTACAGCGAGATCGGCATGAAGGGCG | | | |
| | AGAGGAGGAGGGGCAAGGGCCACGACGGCCTGTACCAGGGCCTG | | | |
| | AGCACCGCCACCAAGGACACCTACGACGCCCTGCACATGCAGGCC | | | |
| | CTGCCCCCCAGGTAA | | | |
| CAR M6 | GCCACCATGGAGTTCGGCCTGAGCTGGCTGTTCCTGGTGGCCATC | n | a | 79. |
| | CTGAAGGGCGTGCAGTGCAGCAGGGACATCCAGATGACCCAGACC | | | |
| | ACCAGCAGCCTGAGCGCCAGCCTGGGCGACAGGGTGACCATCAGC | | | |
| | TGCAGGGCCAGCCAGGACATCAGCAAGTACCTGAACTGGTACCAG | | | |
| | CAGAAGCCCGACGGCACCGTGAAGCTGCTGATCTACCACACCAGC | | | |
| | AGGCTGCACAGCGGCGTGCCCAGCAGGTTCAGCGGCAGCGGCAG | | | |
| | CGGCACCGACTACAGCCTGACCATCAGCAACCTGGAGCAGGAGGA | | | |
| | CATCGCCACCTACTTCTGCCAGCAGGGCAACACCCTGCCCTACACC | | | |
| | TTCGGCGGCGGCACCAAGCTGGAGCTGAAGAGGGGCGGTGGAGG | | | |
| | TTCCGGCGGTGGCGGTTCCGGAGGCGGTGGGTCAGGAGGTGGA | | | |
| | GGCTCCGAGGTGCAGCTGCAGCAGAGCGGCCCCGGCCTGGTGGC | | | |
| | CCCCAGCCAGAGCCTGAGCGTGACCTGCACCGTGAGCGGCGTGA | | | |
| | GCCTGCCCGACTACGGCGTGAGCTGGATCAGGCAGCCCCCCAGGA | | | |
| | AGGGCCTGGAGTGGCTGGGCGTGATCTGGGGCAGCGAGACCACC | | | |
| | TACTACAACAGCGCCCTGAAGAGCAGGCTGACCATCATCAAGGAC | | | |
| | AACAGCAAGAGCCAGGTGTTCCTGAAGATGAACAGCCTGCAGACC | | | |
| | GACGACACCGCCATCTACTACTGCGCCAAGCACTACTACTACGGC | | | |
| | GGCAGCTACGCCATGGACTACTGGGGCCAGGGCACCACCGTGACC | | | |
| | GTGAGCAGCGGTGGCGGTGGAAGTGCCAAGCCCAGCGCCCCCGT | | | |
| | GGTGAGCGGCCCCGCCGCCAGGGCCACCCCCCAGCACACCGTGA | | | |
| | GCTTCACCTGCGAGAGCCACGGCTTCAGCCCCAGGGACATCACCC | | | |
| | TGAAGTGGTTCAAGAACGGCAACGAGCTGAGCGACTTCCAGACCA | | | |
| | ACGTGGACCCCGTGGGCGAGAGCGTGAGCTACAGCATCCACAGCA | | | |
| | CCGCCAAGGTGGTGCTGACCAGGGAGGACGTGCACAGCCAGGTG | | | |
| | ATCTGCGAGGTGGCCCACGTGACCCTGCAGGGCGACCCCCTGAG | | | |
| | GGGCACCGCCAACCTGAGCGAGACCATCAGGGTGCCCCCCACCCT | | | |
| | GGAGGTGACCCAGCAGCCCGTGAGGGCCGAGAACCAGGTGAACG | | | |
| | TGACCTGCCAGGTGAGGAAGTTCTACCCCCAGAGGCTGCAGCTGA | | | |
| | CCTGGCTGGAGAACGGCAACGTGAGCAGGACCGAGACCGCCAGC | | | |
| | ACCGTGACCGAGAACAAGGACGGCACCTACAACTGGATGAGCTGG | | | |
| | CTGCTGGTGAACGTGAGCGCCCACAGGGACGACGTGAAGCTGAC | | | |
| | CTGCCAGGTGGAGCACGACGGCCAGCCCGCCGTGAGCAAGAGCC | | | |
| | ACGACCTGAAGGTGAGCGGCGGTGGCGGCAGCTTCTGGGTGCTG | | | |
| | GTGGTGGTGGGCGGCGTGCTGGCCTGCTACAGCCTGCTGGTGAC | | | |
| | CGTGGCCTTCATCATCTTCTGGGTGAGGAGCAAGAGGAGCAGGCT | | | |
| | GCTGCACAGCGACTACATGAACATGACCCCCAGGAGGCCCGGCCC | | | |
| | CACCAGGAAGCACTACCAGCCCTACGCCCCCCCCAGGGACTTCGC | | | |
| | CGCCTACAGGAGCAGGGTGAAGTTCAGCAGGAGCGCCGACGCCC | | | |
| | CCGCCTACCAGCAGGGCCAGAACCAGCTGTACAACGAGCTGAACC | | | |
| | TGGGCAGGAGGGAGGAGTACGACGTGCTGGACAAGAGGAGGGGC | | | |
| | AGGGACCCCGAGATGGGGGGCAAGCCCAGGAGGAAGAACCCCCA | | | |
| | GGAGGGCCTGTACAACGAGCTGCAGAAGGACAAGATGGCCGAGG | | | |
| | CCTACAGCGAGATCGGCATGAAGGGCGAGAGGAGGAGGGGCAAG | | | |
| | GGCCACGACGGCCTGTACCAGGGCCTGAGCACCGCCACCAAGGAC | | | |
| | ACCTACGACGCCCTGCACATGCAGGCCCTGCCCCCCAGGTAA | | | |
| IgG1 hinge | EPKSPDKTHTCPPCP | a | h | 80. |
| IgG2 hinge | ERKCCVECPPCP | a | h | 81. |
| IgG3 hinge | ELKTPLGDTHTCPRCP | a | h | 82. |
| IgG4 hinge | ESKYGPPCPPCP | a | h | 83. |
| linker C2 | ETIRESKYGPPCPPCPGGGGSVP | a | a | 84. |

TABLE 2
Table 1. Cell surface marker expression patterns used for
cell phenotype and T cell memory phenotype analysis.
Cell phenotypes

| | | |
|---|---|---|
| T cells | CD3+ | CD56− |
| NKT cells | CD3+ | CD56+ |
| NK cells | CD3− | CD56+ |
| Other cells | CD3− | CD56− |

Memory phenotypes

| | | | | |
|---|---|---|---|---|
| Naïve | CD95− | CD45RO− | CD45RA+ | CD27+ |
| SCM¹ | CD95+ | CD45RO− | CD45RA+ | CD27+ |
| SCM-like¹ | CD95+ | CD45RO+ | CD45RA+ | CD27+ |
| CM¹ | CD95+ | CD45RO+ | CD45RA− | CD27+ |
| EM² | CD95+ | CD45RO+ | CD45RA− | CD27− |
| Eff² | CD95+ | CD45RO+ | CD45RA+ | CD27− |

¹Early memory
²Effector

1. A chimeric antigen receptor (CAR) comprising an extracellular spacer which comprises at least one Ig-like C1 domain of signal-regulatory protein alpha (SIRP-alpha) or its fragment or its variant.

2. The CAR according to claim 1, wherein the Ig-like C1 domain of SIRP-alpha is (i) type 1 domain according to SEQ ID NO 1 or its fragment or its variant; or (ii) type 2 domain according to SEQ ID NO 2 or its fragment or its variant.

3. The CAR according to claim 1, wherein the extracellular spacer comprises Ig-like C1 type 1 domain and Ig-like C1 type 2 domain of SIRP-alpha.

4. The CAR according to claim 1, wherein the extracellular spacer further comprises at least one multimerization domain.

5. The CAR according to claim 4, wherein the multimerization domain is selected or multiple multimerization domains are selected from IgG1 hinge region according to SEQ ID NO 4 or SEQ ID NO 80, IgG2 hinge region according to SEQ ID NO 81, IgG3 hinge region according to SEQ ID NO 82, IgG4 hinge region according to SEQ ID NO 83 and/or extracellular domain of CD28 according to SEQ ID NO 3 and/or their fragment and variants.

6. The CAR according to claim 4, wherein the multimerization domain is selected or multiple multimerization domains are selected from IgG1 hinge region according to SEQ ID NO 4 or its fragment and/or extracellular domain of CD28 according to SEQ ID NO 3 or its fragment.

7. The CAR according to claim 4, wherein the multimerization domain is selected or multiple multimerization domains are selected from IgG4 hinge region according to SEQ ID NO 83 or its fragment and/or extracellular domain of CD28 according to SEQ ID NO 3 or its fragment.

8. The CAR according to claim 1, wherein the extracellular spacer locates between a transmembrane domain and an antigen binding domain and connects them.

9. The CAR according to claim 8, wherein the antigen binding domain comprises a single chain variable fragment (scFv).

10. The CAR according to claim 1, wherein the spacer dimerizes CAR at least with one disulfide bridge.

11. A CAR comprising an extracellular spacer comprising an amino acid sequence according to SEQ ID NO 10, SEQ ID NO 11, SEQ ID NO 12, SEQ ID NO 13, SEQ ID NO 14, SEQ ID NO 15, SEQ ID NO 16, SEQ ID NO 17, SEQ ID NO 18, SEQ ID NO 56, SEQ ID NO 57, SEQ ID NO 58, SEQ ID NO 59, SEQ ID NO 60, or SEQ ID NO 61.

12. A chimeric antigen receptor (CAR) comprising (i) an extracellular spacer according to claim 1, (ii) an antigen binding domain, (iii) a transmembrane domain, (iv) an intracellular signaling domain, and (v) optionally a costimulatory domain.

13. The CAR according to claim 12, wherein the antigen binding domain comprises an antibody or its fragment.

14. The CAR according to claim 12, wherein the antigen binding domain comprises a single chain variable fragment (scFv).

15. The CAR according to claim 12, wherein the antigen binding domain targets a tumor antigen.

16. The CAR according to claim 15, wherein the tumor antigen is CD19 or HER-2.

17. The CAR according to claim 12, wherein the transmembrane domain comprises a transmembrane domain of CD28 according to SEQ ID NO 23.

18. The CAR according to claim 12, wherein the intracellular signaling domain and/or co-stimulatory domain comprises intracellular domain of CD3zeta according to SEQ ID NO 25 or its fragment and/or intracellular domain of CD28 according to SEQ ID NO 24 or its fragment.

19. A chimeric antigen receptor (CAR) comprising
(i) a single chain variable fragment (scFv);
(ii) IgG hinge domain;
(iii) Ig-like C1 type 1 and/or Ig-like C1 type 2 domain of signal-regulatory protein alpha-1;
(iv) CD3zeta;
(v) CD28 transmembrane domain; and
(vi) optionally CD28 extracellular domain and/or CD28 intracellular domain.

20. A CAR comprising an amino acid sequence according to SEQ ID NO 26, SEQ ID NO 27, SEQ ID NO 28, SEQ ID NO 29 SEQ ID NO 30, SEQ ID NO 31, SEQ ID NO 32, SEQ ID NO 33, SEQ ID NO 34, SEQ ID NO 54, SEQ ID NO 62, SEQ ID NO 63, SEQ ID NO 64, SEQ ID NO 65, SEQ ID NO 66, or SEQ ID NO 67.

21. A polynucleotide encoding a CAR of claim 1.

22. A vector comprising the polynucleotide of claim 21.

23. A cell comprising a CAR according to claim 1.

24. A cell according to claim 23, wherein the cell is a T-cell

25. A method to adjust the length of chimeric antigen receptor (CAR) by selecting at least two domains from group (i) IgG hinge domain, (ii) Ig-like C1 type 1 domain of signal-regulatory protein alpha-1, (iii) Ig-like C1 type 2 domain of signal-regulatory protein alpha-1, or (iv) CD28 extracellular fragment to the spacer domain resulting in chimeric antigen receptors with different lengths.

26. The method according to claim 25, wherein the spacer domain does not bind or has reduced binding affinity to Fc receptor.

27. A cell comprising a polynucleotide of claim 21.

28. The cell according to claim 27, wherein the cell is a T-cell.
